(19) United States
(12) Patent Application Publication
(10) Pub. No.: US 2005/0282233 A1
(43) Pub. Date: Dec. 22, 2005

(54) Multivalent antibody materials and methods for VEGF/PDGF family of growth factors

(75) Inventors: Ulf Eriksson, Stockholm (SE); Kari Alitalo, Helsinki (FI); Marc Gregory Achen, Parkville (AU); Christoph Renner, Homburg/Saar (DE); Steven Stacker, Parkville (AU); Hong Li, Stockholm (SE); Pirjo Laakkonen, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(73) Assignees: LUDWIG INSTITUTE FOR CANCER RESEARCH, New York, NY (US); LICENTIA, LTD., Helsinki (FI)

(21) Appl. No.: 11/075,400

(22) Filed: Mar. 7, 2005

Related U.S. Application Data
(60) Provisional application No. 60/550,511, filed on Mar. 5, 2004.
(60) Provisional application No. 60/586,662, filed on Jul. 9, 2004.

(51) Int. Cl. G01N 33/53; G01N 33/567; C07K 16/22

(52) U.S. Cl. 435/7.2; 530/388.25

(57) ABSTRACT

The present invention relates to materials and methods for modulating angiogenesis. The compositions of the invention provide antibody substances specific for two or more PDGF/VEGF family members, which are useful for modulating angiogenesis and lymphangiogenesis in a subject.

The present application claims the priority benefit of U.S. Provisional Application No. 60/550,511, filed Mar. 5, 2004, and U.S. Provisional Application No. 60/586,662, filed Jul. 9, 2004. All priority applications are incorporated herein by reference.

### FIELD OF THE INVENTION

The present invention relates to novel therapeutics agents, formulations, and methods to inhibit angiogenesis in patients experiencing aberrant angiogenesis. In particular, the invention provides crossreacting, multivalent bispecific antibodies that react with two or more ligands in the PDGF/VEGF family of growth factors.

### BACKGROUND OF THE INVENTION

Angiogenesis is a fundamental process required for normal growth and development of tissues, and involves the proliferation of new capillaries from pre-existing blood vessels. Angiogenesis is not only involved in embryonic development and normal tissue growth, repair, and regeneration, but is also involved in the female reproductive cycle, establishment and maintenance of pregnancy, and in repair of wounds and fractures. In addition to angiogenesis which takes place in the healthy individual, angiogenic events are involved in a number of pathological processes, notably tumor growth and metastasis, and other conditions in which blood vessel proliferation, especially of the microvascular system, is increased, such as diabetic retinopathy, psoriasis and arthropathies. Inhibition of angiogenesis is useful in preventing or alleviating these pathological processes.

Because of the crucial role of angiogenesis in so many physiological and pathological processes, factors involved in the control of angiogenesis have been intensively investigated. A number of growth factors have been shown to be involved in the regulation of angiogenesis; these include fibroblast growth factors (FGFs), platelet-derived growth factor (PDGF), transforming growth factor α (TGFα), and hepatocyte growth factor (HGF). See for example Folkman et al, “Angiogenesis”, J. Biol. Chem., 1992 267 10931-10934 for a review.

It has been suggested that a particular family of endothelial cell-specific growth factors and their corresponding receptors is primarily responsible for stimulation of endothelial cell growth and differentiation, and for certain functions of the differentiated cells. These factors are members of the PDGF/VEGF family, and appear to act via receptor tyrosine kinases (RTKs).

To date a number of PDGF/VEGF family members have been identified. These include PDGF-A (see e.g., GenBank Acc. No. X06374), PDGF-B (see e.g., GenBank Acc. No. M12783), PDGF-C (Intl. Publ. No. WO 00/18212), PDGF-D (Intl. Publ. No. WO 00/027879), VEGF (also known as VEGF-A or by particular isoform), Placenta growth factor, PlGF (U.S. Pat. No. 5,919,899), VEGF-B (also known as VEGF-related factor (VRF) Intl. Publ. No. PCT/US96/02597 and WO 96/26736), VEGF-C, (U.S. Pat. No. 6,221,839 and WO 98/33917), VEGF-D (also known as c-fos-induced growth factor (FIGF) (U.S. Pat. No. 6,235,713, Intl. Publ. No. WO98/07832), VEGF-E (also known as NZ7 VEGF or OV NZ7; Intl. Publ. No. WO00/025805 and U.S. Patent Publ. No. 2003/0113870), NZ2 VEGF (also known as OV NZ2; see e.g., GenBank Acc. No. S67520), D1701 VEGF-like protein (see e.g., GenBank Acc. No. AF106020; Meyer et al., EMBO J. 18:363-374), and NZ10 VEGF-like protein (described in Intl. Patent Application PCT/US99/25869) [Stacker and Achen, Growth Factors 17:1-11 (1999); Neufeld et al., FASEB J 13:9-22 (1999); Ferrara, J Mol Med 77:527-543 (1999)].

Vascular endothelial growth factor (VEGF/VEGF-A) is a homodimeric glycoprotein that has been isolated from several sources. VEGF shows highly specific mitogenic activity for endothelial cells. VEGF has important regulatory functions in the formation of new blood vessels during embryonic vasculogenesis and in angiogenesis during adult life (Carmeliet et al., Nature, 380: 435-439, 1996; Ferrara et al., Nature, 380: 439-442, 1996; reviewed in Ferrara and Davis-Smyth, Endocrine Rev., 18: 4-25, 1997). The significance of the role played by VEGF has been demonstrated in studies showing that inactivation of a single VEGF allele results in embryonic lethality due to failed development of the vasculature (Carmeliet et al., Nature, 380: 435-439, 1996; Ferrara et al., Nature, 380: 439-442, 1996). In addition VEGF has strong chemoattractant activity towards monocytes, can induce the plasminogen activator and the plasminogen activator inhibitor in endothelial cells, and can also induce microvascular permeability. Because of the latter activity, it is sometimes referred to as vascular permeability factor (VPF). The isolation and properties of VEGF have been reviewed; see Ferrara et al., J. Cellular Biochem., 47: 211-218, 1991 and Connolly, J. Cellular Biochem., 47: 219-223, 1991. Alternative mRNA splicing of a single VEGF gene gives rise to five isoforms of VEGF.

VEGF-B has similar angiogenic and other properties to those of VEGF, but is distributed and expressed in tissues differently from VEGF. In particular, VEGF-B is very strongly expressed in heart, and only weakly in lung, whereas the reverse is the case for VEGF. This suggests that VEGF and VEGF-B, despite the fact that they are co-expressed in many tissues, may have functional differences.

PlGF was isolated from a term placenta cDNA library. Its isolation and characteristics are described in detail in Maglione et al., Proc. Natl. Acad. Sci. USA, 88: 9267-9271, 1991. Presently its biological function is not well understood.

VEGF-C was isolated from conditioned media of PC-3 prostate adenocarcinoma cell line (CRL1435) by selecting for a component of the medium that caused tyrosine phosphorylation of the endothelial cell-specific receptor tyrosine kinase Flt4 (VEGFR-3), using cells transfected to express Flt4. VEGF-C isolation and characteristics are described in detail in Joukov et al, EMBO J. 15 290-298, 1996 and U.S. Pat. Nos. 6,221,839; 6,235,713; 6,361,946; 6,403,088; and 6,645,933 and International Patent Publ. Nos. WO 97/05250, WO 98/07832, and WO 98/01973, incorporated herein by reference. In mouse embryos, VEGF-C mRNA is expressed primarily in the allantois, jugular area, and the metanephros. (Joukov et al., J Cell Physiol 173:211-215, 1997), and appears to be involved in the regulation of lymphatic angiogenesis (Jeltsch et al., Science, 276:1423-1425, 1997).

VEGF-D was isolated and described in detail in International Patent Application No. PCT/US97/14696 (WO98/07832). The VEGF-D gene is broadly expressed in the adult human, but is not ubiquitously expressed. VEGF-D is strongly expressed in heart, lung and skeletal muscle. Intermediate levels of VEGF-D are expressed in spleen, ovary, small intestine and colon, and a lower expression occurs in kidney, pancreas, thymus, prostate and testis. No VEGF-D mRNA was detected in RNA from brain, placenta, liver or peripheral blood leukocytes.

PDGF-C is described in Intl. Patent Publ. No. WO 00/18212. The PDGF-C polypeptide exhibits a unique protein structure compared to other VEGF/PDGF family members. PDGF-C possesses a CUB domain in the N-terminal region, which is not present in other family members, and also possesses a three amino acid insert (NCA) between conserved cysteines 3 and 4 in the VEGF homology domain. The VHD of PDGF-C most closely resembles that of VEGF-C and VEGF-D. PDGF-C mRNA expression was highest in heart, liver, kidney, pancreas, and ovaries, and expressed at lower levels in most other tissues, including placenta, skeletal muscle and prostate. A truncated form of PDGF-C containing the VHD binds to the PDGF-alpha receptor.

PDGF-D is described in Intl. Patent Publ. No. WO 00/027879 and WO 00/125437. Similar to PDGF-C, PDGF-D possesses a CUB domain and a three amino acid insert (NCA) between conserved cysteines 3 and 4 in the VEGF homology domain. Additionally, the invariant fifth cysteine found in the other members of the PDGF/VEGF family is not conserved in PDGF-D. This feature is unique to PDGF-D. The VHD of PDGF-D most closely resembles that of VEGF-C and VEGF-D. PDGF-D mRNA expression was highest in heart, ovary and pancreas, and expressed at lower levels in testis, kidney, liver, placenta, prostate and small intestine.

Vascular endothelial growth factors appear to act by binding to receptor tyrosine kinases of the PDGF-receptor family. Seven receptor tyrosine kinases have been identified, namely Flt-1 (VEGFR-1), KDR/Flk-1 (VEGFR-2), Flt4 (VEGFR-3), PDGFR-α, PDGFR-β, Tie and Tek/Tie-2. All of these have the intrinsic tyrosine kinase activity which is necessary for signal transduction. The essential, specific role in vasculogenesis and angiogenesis of Flt-1, Flk-1, Tie and Tek/Tie-2 has been demonstrated by targeted mutations inactivating these receptors in mouse embryos. Overexpression of either the VEGF/PDGF family of growth factors or VEGF/PDGF receptors can lead to aberrant development of the vasculature system (Saaristo et al., FASEB J. 16:1041-9, 2002; Kubo et al., Proc Natl Acad Sci USA. 99:8868-73, 2002.). The activity of VEGF/VEGFR also promotes angiogenesis of new cells and developing tissue, thereby facilitating the angiogenesis and vascularization of tumor cells.

Although therapies directed to blockade of VEGF/PDGF signaling through their receptors has shown promise for inhibition of angiogenesis and tumor growth, a need exists for more effective and complete therapies.

### SUMMARY OF THE INVENTION

The present invention relates to novel compositions and methods of use thereof for the inhibition of aberrant angiogenesis and lymphangiogenesis in cells, and inhibition of other effects of members of the PDGF/VEGF family of growth factors. The compositions of the invention provide antibody substances, antibodies and polypeptides specific for two or more PDGF/VEGF molecules. Administration of the compositions of the invention to patients inhibits growth factor stimulation of PDGFR- and/or VEGFR-mediated angiogenesis and lymphangiogenesis.

In one aspect, the invention is an antibody substance that specifically binds to first and second growth factors selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E), human placental growth factor (PlGF), human platelet-derived growth factor-A (PDGF-A), human platelet-derived growth factor-B (PDGF-B) human platelet-derived growth factor-C (PDGF-C), and human platelet-derived growth factor-D (PDGF-D).

In one aspect, the invention is an antibody substance that specifically binds to first and second growth factors selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E), human placental growth factor (PlGF), human platelet-derived growth factor-A (PDGF-A), human platelet-derived growth factor-B (PDGF-B) human platelet-derived growth factor-C (PDGF-C), and human platelet-derived growth factor-D (PDGF-D), wherein each of the growth factors binds and stimulates phosphorylation of at least one receptor tyrosine kinase, and wherein the antibody substance inhibits the first and second growth factors to which it binds from stimulating phosphorylation of the receptor tyrosine kinases.

An “antibody substance” as used herein refers to any antibody or molecule comprising all or part of an antigen-binding site of an antibody and that retains immunospecific binding of the original antibody. Antibody-like molecules such as lipocalins that do not have CDRs but that behave like antibodies with specific binding affinity for PDGF/VEGF growth factors also can be used to practice this invention and are considered part of the invention. Antibody substances of the invention include monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention, fragments of the foregoing, and polypeptide molecules that include antigen binding portions and retain antigen binding properties. As described herein, antibody substances can be derivitized with chemical modifications, glycosylation, and the like and retain antigen binding properties.

Binding specificity refers to the well known property of antibodies to exhibit immunospecific binding interactions whereby the antibody substance differentially binds and recognizes its specified antigen with an affinity measurably greater than it cross-reacts with other antigens. Specificity for any particular growth factor is described below in greater detail. Generally speaking, the target family of growth factors all exist in at least one form (isoform, processed form) that circulates in the bloodstream and that stimulates one or more PDGFR/VEGFR target receptors, yet most of the growth factors exist in additional isoforms or partly processed forms or pro-forms as well. Antibodies that are “specific” for a particular growth factor are antibodies that immunospecifically recognize a circulating, active form of the growth factor. Preferably, the specific antibodies immunospecifically bind other forms of the growth factors as well. By way of example, VEGF-A exists in multiple isoforms, some of which circulate and others of which associate with heparin sulfate proteoglycans on cell surfaces. Antibodies that are specific for VEGF-A bind to at least a circulating isoform, preferably all circulating isoforms, and more preferably, bind other major isoforms as well. By way of another example, VEGF-C is translated as a prepro-molecule with extensive amino-terminal and carboxy-terminal propeptides that are cleaved to yield a “fully processed” form of VEGF-C that binds and stimulates VEGFR-2 and VEGFR-3. Antibodies specific for VEGF-C bind to at least the fully processed form of VEGF-C, and preferably also bind to partly processed forms and unprocessed forms.

In still another variation, the invention includes antibody substances that bind to two or more of the aforementioned growth factors, wherein binding to one, two, or more of the growth factors occurs at an epitope wherein binding inhibits extracellular processing of the growth factor into a more mature or active isoform. By way of example, antibodies that bind PDGF-C or -D and prevent or inhibit proteolytic processing of the molecule from a full-length, secreted form into an active form are contemplated.

Additional description is used herein when a more specialized meaning is intended. For example, VEGF-B₁₆₇ is heparin bound whereas VEGF-B₁₈₆ is freely secreted. An antibody of the invention that minimally binds the circulating isoform is said to be specific for VEGF-B, and such an antibody preferably also binds the heparin bound form. An antibody of the invention that is “specific for heparin-bound VEGF-B” or “specific for VEGF-B₁₆₇” is an antibody that differentially recognizes the heparin bound isoform, compared to the freely circulating isoform. An antibody of the invention that is “specific for VEGF-B₁₈₆” is an antibody that differentially recognizes the circulating form, compared to the heparin bound form. Antibodies specific for each isoform of a growth factor are contemplated as components of some embodiments of the antibody substances of the invention.

Antibody substances that are specific for a first and second growth factor are antibody substances that exhibit specificity as described herein for two different members of the genus of growth factors set forth above. (VEGF-C and VEGF-A are considered different members, whereas two isoforms of VEGF-A are not.) As described below in detail, bispecific antibody substances (specific for two antigens) of the invention are generated in at least two ways. First, traditional antibody generation and screening techniques are used to select a single antibody molecule (from a polyclonal library, a monoclonal library, a phage display library, or the like) that exhibits strong immunoreactivity towards two growth factors. For the purposes of this invention, bispecific antibodies generated in this fashion are termed cross-reacting bispecific antibodies. Second, bispecific antibody substances are assembled using recombinant techniques from an antibody substance that is specific for a first growth factor and another antibody substance that is specific for a second growth factor, and the assembled molecules are screened to verify that they retain immunospecificity for the original growth factor antigens. For the purposes of this invention, bispecific antibodies generated in this fashion are termed multivalent bispecific antibodies. The designations “first” and “second” is for ease and clarity in description only, and is not meant to signify a particular order.

Antibody substances that are engineered to bind to three or more (e.g., 4, 5, 6, 7, 8) members of the growth factor genus are said to satisfy the requirement of binding to “first and second” growth factors for the purposes of this invention, and are specifically contemplated.

The statement “wherein each of the growth factors binds and stimulates phosphorylation of at least one receptor tyrosine kinase” is meant simply as an acknowledgement that each member of the growth factor genus described above binds with high affinity to, and stimulates phosphorylation of, at least one PDGF receptor or VEGF receptor (or receptor heterodimer) selected from VEGFR-1, VEGFR-2, VEGFR-3, PDGFR-alpha, and PDGFR-beta. The statement refers to well known properties of the growth factors toward their cognate receptors, and is not meant as a limiting feature per se of the antibody substances of the invention. (For example, VEGF-A has been shown to bind to VEGFR-1 and VEGFR-2 and induce tyrosine phosphorylation of both receptors and initiate downstream receptor signaling.) However, preferred antibody substances of the invention do more than simply bind their target (first and second) growth factors: a preferred antibody substance also inhibits the first and second growth factors to which it binds from stimulating phosphorylation of at least one (and preferably all) of the receptor tyrosine kinases to which the first and second growth factors bind. Stimulation of tyrosine phosphorylation is readily measured using in vitro cell-based assays and anti-phosphotyrosine antibodies. Since phosphorylation of the receptor tyrosine kinases is an initial step in a signaling cascade, it is a convenient indicator of whether the antibody is capable of inhibiting growth factor-mediated signal transduction that leads to cell migration, cell growth, and other responses. As set forth herein, a number of other cell based and in vivo assays can be used to confirm the growth factor neutralizing properties of antibody substances of the invention. For example, positron emission tomography (PET) is useful to determine the effects of antibody administration in vivo (Gambhir et al., Nat Rev. Cancer 2:683-93, 2002). In one aspect, an antibody substance described herein is labeled with a positron emitter, which when bound to its antigen, enables monitoring of protein levels before and after antibody administration, as well as identifying the location of the antibody (Smith-Jones et al., Nat. Biotechnol. 22:701-6, 2004). Positrons emitters useful in the invention include ¹¹C, ¹⁵O, ¹³N, ¹⁸F, ¹⁹F, ⁶⁴Cu, ⁶⁷Cu, or ⁶⁸Ga.

In another aspect, the invention is an antibody substance produced by a process comprising:
(a) screening a library of antibody molecules to identify at least one antibody molecule that binds to a first growth factor selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E) human placental growth factor (PlGF), human platelet-derived growth factor-A (PDGF-A), human platelet-derived growth factor-B (PDGF-B) human platelet-derived growth factor-C (PDGF-C), and human platelet-derived growth factor-D (PDGF-D), wherein each of the growth factors binds and stimulates phosphorylation of at least one receptor tyrosine kinase;
(b) screening molecule(s) identified in step (a) to identify at least one molecule that binds to a second growth factor selected from the group; and
(c) screening molecule(s) identified in step (b) to identify at least one molecule that inhibits the first and second growth factors to which it binds from stimulating phosphorylation of the receptor tyrosine kinases, wherein the antibody substance comprises a molecule identified in step (c).

The library (collection) of antibody molecules may be any collection of antibodies, antibody fragments, antibody variable regions, single chain antibodies and antibody fragments, or other types of antibody substances described herein that assembled or created by available techniques. Exemplary libraries include polyclonal antibodies obtained from animal serum following traditional animal immunization; monoclonal/hybridoma-libraries assembled using known monoclonal antibody techniques; recombinant libraries made from immunoglobulin libraries or other nucleotide constructs such as phage display libraries, bacterial expression libraries, and the like. Commercially available libraries that may be screened include human antibody libraries from Dyax, Corp. (Cambridge, Mass.), and Cambridge Antibody Technologies (Cambridge, UK). Alternatively, antibody libraries may be generated as described in U.S. Pat. No. 6,319,690 or 6,300,064. Making the library is an optional additional step preceding the screening step, in some variations of the invention.

Screening of the antibody library refers to all known techniques for evaluating whether antibody substances bind to a target antigen, including but not limited to ELISA formats, western blot, and antibody arrays. Labeling of the antibody or the antigen often facilitates screening. In preferred embodiments, several molecules are selected in screening step (a) because only a fraction of such molecules are likely to satisfy the second screen of step (b), using a second (different) growth factor. The second screening step can be conducted with all of the same techniques that are available for the first screening step, only using the second (different) growth factor as the antigen.

Not all antibody substances that meet the binding criteria of steps (a) and (b) will necessarily be useful for inhibiting growth factor mediated activation of receptors expressed by cells. Step (c) is a screen to select that subset, using phosphorylation assays and/or biological response assays such as those described herein in detail.

In still another variation, the invention is an antibody substance produced by a process comprising:
(a) screening a library of antibody molecules to identify at least one antibody molecule that binds to a first growth factor selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E) human placental growth factor (PlGF), human platelet-derived growth factor-A (PDGF-A), human platelet-derived growth factor-B (PDGF-B) human platelet-derived growth factor-C (PDGF-C), and human platelet-derived growth factor-D (PDGF-D), wherein each of the growth factors binds and stimulates phosphorylation of at least one receptor tyrosine kinase;
(b) screening a library of antibody molecules to identify at least one molecule that binds to a second growth factor selected from the group;
(c) fusing an antigen binding domain of an antibody molecule identified in step (a) with an antigen binding domain of an antibody molecule identified in step (b) to make antibody fusions, and
(d) screening the antibody fusions to identify at least one molecule that inhibits the first and second growth factors to which it binds from stimulating phosphorylation of the receptor tyrosine kinases, wherein the antibody substance comprises an antibody fusion identified in step (d).

In this variation of the invention, screening steps (a) and (b) can be completely independent of each other, i.e., they can be performed in either order with the same or different libraries of antibody substances. Molecules that satisfy screening step (a) and molecules that satisfy screening step (b) are fused to each other using any available technique in advance of the further screening and selection specified in step (c). The molecules can be fused with a peptide bond, directly or indirectly (with the use of peptide linkers or spacers). The molecules can be fused using disulfide bridges, using complementary binding partners (one high-affinity binding partner fused to molecules from step (a) and complementary binding partner fused to molecules of step (b), using chemical attachment techniques, taking advantage of natural assembly of antibody chains into antibodies, or other techniques known in the art, described herein, or discovered.

The screening step (d) may involve a direct phosphorylation assay or indirect activity assay (cell migration, cell growth, etc.) that provides evidence of ligand-mediated stimulation of a receptor, as described elsewhere herein.

In some variations, the antibody substance comprises an antibody variable region of an antibody that binds the first growth factor attached to an antibody variable region of an antibody that binds the second growth factor. In some variations, the antibody substance comprises antibody heavy and light chain variable regions of an antibody that binds to the first growth factor attached to antibody heavy and light chain variable regions of an antibody that bind to the second growth factor. The antibody heavy and light chain variable regions that bind to the first growth factor can be attached to each other to form a single polypeptide; and the antibody heavy and light chain variable regions that bind to the second growth factor can be attached to each other to form a single polypeptide.

The antibody heavy and light chain variable regions of the antibody specific for a growth factor may be attached to form a single polypeptide using any method known in the art, such as chemical crosslinking, addition of linker peptides, or addition of oligo- or polypeptides, as described herein.

In some variations, the antibody substance that is a single polypeptide comprises the antigen binding portions of the antibody substance. In some variations, the antibody substance comprises a F(ab) antibody fragment that binds to the first growth factor attached to a F(ab) antibody fragment that binds to the second growth factor. In other variations, the antibody substance comprises a F(ab)₂ fragment that binds to the first growth factor attached to a F(ab)₂ fragment that binds to the second growth factor. In still another variation, the single polypeptide may be a single domain antibody that binds the first and second growth factors. It is contemplated that the single domain is a heavy chain variable domain, (V_{H}) or a light chain variable domain (V_{L}).

In some variations, the antibody substance of the invention is a monoclonal antibody. The monoclonal antibody may be generated by any technique known in the art or discovered for generating monoclonal antibodies, by fusion of hydridomas to generate a hybrid hybridoma, or by genetic manipulation to generate a monoclonal antibody that is multivalent. In a related aspect, the invention includes a hybridoma which expresses the monoclonal antibody of the invention.

In one variation the antibody is a chimeric antibody. In preferred variations, the antibody substance is a humanized antibody or a human antibody. For example, in one variation, the antibody substance comprises: (a) a humanized or human antibody heavy chain variable region of an antibody that binds to the first growth factor; (b) a humanized or human antibody light chain variable region of an antibody that binds to the first growth factor; (c) a humanized or human antibody heavy chain variable region of an antibody that binds to the second growth factor; and (d) a humanized or human antibody light chain variable region of an antibody that binds to the second growth factor. Optionally, the antibody substance further comprises human antibody constant regions. The human light chain constant regions may comprises either the kappa light chain region or the lambda light chain region. The human heavy chain constant regions are preferably selected from the group consisting of an IgM constant region, an IgG constant region, an IgA constant region, an IgD constant region, or an IgE constant region. In a preferred embodiment, the human heavy chain constant region is from an IgG antibody, wherein the IgG is selected from the group consisting of IgG1, IgG2, IgG3, or IgG4.

In another variation, the antibody substance comprises a first leucine zipper linked to an antigen binding site that binds the first growth factor, and a second leucine zipper linked to an antigen binding site that binds to the second growth factor, wherein the leucine zippers dimerize to form the antibody substance. For example, the leucine zippers may be derived from either the Fos protein the Jun protein or the c-myc gene product. It is contemplated that the leucine zipper creates a dimerization interface wherein proteins containing leucine zippers may form stable homodimers and/or heterodimers. Thus, in the present invention leucine zippers that are attached to antigen specific regions of multiple specificities dimerize to form a multivalent antibody substance.

The antibody substances of the invention can be selected or engineered to bind two, three, four, five, six, or more of the target growth factors. In one variation, the antibody substances that specifically binds a first and second growth factor are bispecific antibody substances. Bispecific antibodies to all permutations of two growth factors from the list provided herein are intended as embodiments of the invention. Preferred bispecific antibodies include anti-VEGF-A/VEGF-E-specific antibodies, anti-VEGF-C/VEGF-D-specific antibodies, anti-PDGF-C/PDGF-D-specific antibodies, anti-VEGF-B/PlGF-specific, antibodies, anti-VEGF-A/VEGF-B-specific antibodies; and anti-VEGF-A/VEGF-D antibodies.

Anti-VEGF-A/Anti-PDGF antibodies are a preferred bispecific or multivalent antibody, especially for cancer indications. Such antibody substances are expected to show promise with tumors that shrink in response to an anti-VEGF-A therapy (e.g., Avastin™, Genentech) but are not eliminated and subsequently increase again in size.

In one embodiment, the invention provides a bispecific antibody substance comprising a first antigen binding site that specifically binds to the first growth factor that is VEGF-B, and a second antigen binding site that specifically binds to the second growth factor that is PlGF, wherein the bispecific antibody substance inhibits VEGF-B-mediated and PlGF-mediated phosphorylation of VEGFR-1.

In another embodiment, the invention provides a bispecific antibody substance comprising a first antigen binding site that specifically binds to the first growth factor that is VEGF-A, and a second antigen binding site that specifically binds to the second growth factor that is VEGF-B, wherein the bispecific antibody substance inhibits VEGF-A-mediated and VEGF-B-mediated phosphorylation of VEGFR-1. Preferably, such a bispecific antibody substance also inhibits VEGF-A-mediated phosphorylation of VEGFR-2.

In one aspect, the VEGF-A/VEGF-B bispecific antibody substance binds to an epitope comprised of amino acids 87-110 of VEGF-A (SEQ ID NO: 23), amino acids 82-105 of VEGF-B (SEQ ID NO: 24), or a fragment thereof. In a related aspect, the fragment may be a fragment of at least 5, 6, 7, 8, 9, 10, 11, 12, 13, 14 or 15 consecutive amino acids derived from the VEGF-A or VEGF-B amino acid sequences of SEQ ID NO: 23 or 24. A sequence derived from or derivative from the region of similarity comprises any sequence that is identical to the identified region of similarity, or a peptide sequence containing conservative substitution mismatches within the selected region which do not interfere with cross-reactivity of the bispecific antibody.

In a further embodiment, the invention provides a bispecific antibody substance comprising a first antigen binding site that specifically binds to the first growth factor that is VEGF-C and a second antigen binding site that specifically binds to the second growth factor that is VEGF-D, wherein the bispecific antibody substance inhibits VEGF-C-mediated and VEGF-D-mediated phosphorylation of VEGFR-3. In a related embodiment, the bispecific antibody substance comprises a first antigen binding site that specifically binds to the first growth factor that is VEGF-C and a second antigen binding site that specifically binds to the second growth factor that is VEGF-D, wherein the bispecific antibody substance inhibits VEGF-C-mediated and VEGF-D-mediated phosphorylation of VEGFR-2. Preferred VEGF-C/VEGF-D bispecific antibodies inhibit phosphorylation of both receptors by both ligands.

In one variation, the VEGF-C/VEGF-D bispecific antibody binds at an epitope to inhibit extracellular processing of the N-terminal pro-peptides of VEGF-C and/or VEGF-D. Such inhibition prevents or inhibits creation of fully processed forms of these growth factors that bind VEGFR-2 and VEGFR-3.

In another embodiment, the invention provides a bispecific antibody substance comprising a first antigen binding site that specifically binds to the first growth factor that is VEGF-A, and a second antigen binding site that specifically binds to the second growth factor that is VEGF-E, wherein the bispecific antibody substance inhibits VEGF-A-mediated and VEGF-E-mediated phosphorylation of VEGFR-2.

In a further embodiment, the invention contemplates a bispecific antibody substance comprising a first antigen binding site that specifically binds to the first growth factor that is PDGF-A, PDGF-B, PDGF-C or PDGF-D and a second antigen binding site that specifically binds to the second growth factor that is PDGF-A, PDGF-B, PDGF, C or PDGF-D. In a related embodiment, the invention contemplates a bispecific antibody substance comprising a first antigen binding site that specifically binds to the first growth factor that is PDGF-C and a second antigen binding site that specifically binds to the second growth factor that is PDGF-D, wherein the bispecific antibody substance inhibits PDGF-C-mediated and PDGF-D-mediated phosphorylation of PDGF receptors to which these growth factors bind. In some embodiments, it is contemplated that the PDGF-C/PDGF-D bispecific antibody inhibits or neutralizes PDGF activity by interfering with binding of the ligand with its receptor. In some embodiments the bispecific antibody prevents or inhibits processing of the PDGF-C and PDGF-D proteins to their active forms, thereby neutralizing and blocking activation of the proteins.

In one aspect, the bispecific antibody substance binds to an epitope comprised of amino acids 231-274 of PDGF-C (SEQ ID NO: 27), amino acids 255-296 of PDGF-D (SEQ ID NO: 28), or a fragment thereof. In a related aspect, the fragment may be a fragment at least 5, 6, 7, 8, 9, 10, 11, 12, 13, 14 or 15 consecutive amino acids derived from PDGF-C or PDGF-D set out in SEQ ID NO: 27 or 28. In a related aspect, the bispecific antibody substance binds to an epitope comprised of amino acids 255-272 of PDGF-D (SEQ ID NO: 29), or a fragment thereof. In an additional aspect, the bispecific antibody substance binds to an epitope comprised of amino acids 231-250 of PDGF-C (SEQ ID NO: 32). In a further aspect, the bispecific antibody substance binds an epitope comprised of any one of the SEQ ID NO: 27, SEQ ID NO: 28, SEQ ID NO: 31, SEQ ID NO: 32 or a fragment thereof, wherein the epitope further comprises additional amino acids at either the N- or C-terminal end. It is contemplated that the additional amino acids may comprise 1, 2, 3, 4, 5, 6, 7, or up to 10 amino acids added at either end of the epitope.

It is contemplated that the bispecific antibody substances of the invention may be fused to detectable labels to facilitate detection of the antibody or cell, to toxins to promote cell death, or to prodrugs to facilitate delivery of therapeutics. For example, the antibody substances of the invention may be fused to a label. Exemplary labels include, but are not limited to, fluorescent labels, radionucleotides, enzymes, and other detectable labels described herein. Exemplary cytotoxins contemplated for fusion to antibodies of the invention include, but are not limited to radionuclides, such as Iodine 131, Ytterium 90, Rhenium 186, Rhenium-188, Bismuth-213, lutetium 177, ³²P, strontium-89, and Samarium-153; and cytotoxins, such as DM1, a derivative of maytansine, auristatin, duocarmycin, fullerene, and calicheamicin. Additionally, the antibodies of the invention may be fused to prodrugs which are converted to active drugs in vivo.

In another embodiment, the invention includes a composition comprising an antibody substance according to the invention in a pharmaceutically acceptable carrier. Exemplary, medically accepted pharmaceutically acceptable carriers are identified below.

In a related aspect, the invention includes the use of an antibody substance, antibody or polypeptide of the invention for inhibition of angiogenesis or lymphangiogenesis, or for the manufacture of a medicament for inhibition of angiogenesis or lymphangiogenesis, or for other uses described herein.

In another aspect, the invention includes use of an antibody substance of the invention specific for PDGF molecules for the inhibition of fibrosis, or for manufacture of a medicament for inhibition of fibrosis. Use of antibodies bispecific for PDGF molecules, including PDGF-A, PDGF-B, PDGF-C or PDGF-D, are contemplated by the invention. In a preferred embodiment, antibodies specific for PDGF-C and PDGF-D are useful to inhibit fibrosis. In a further aspect of the invention, antibody substances of the invention are used to prevent heterodimerization between ligands of the PDGF/VEGF family of growth factors that are capable of dimerizing.

In another aspect, the invention includes use of an antibody substance of the invention for imaging, or for manufacture of a medicament for imaging. Imaging may be performed in vitro, e.g., on a tissue or other biological sample removed from a mammalian subject, or may be performed in vivo. In either circumstance, the antibody substance of the invention preferably is coupled to a detectable label to facilitate the imaging.

In a further aspect, the invention is an isolated polynucleotide comprising a nucleotide sequence that encodes any one of the antibody substances described herein. For example, in one embodiment, the invention provides a polynucleotide comprising a nucleotide sequence that encodes an antibody substance that specifically binds to first and second growth factors selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E) human placental growth factor (PlGF), human platelet-derived growth factor-A (PDGF-A), human platelet-derived growth factor-B (PDGF-B) human platelet-derived growth factor-C (PDGF-C), and human platelet-derived growth factor-D (PDGF-D), wherein each of the growth factors binds and stimulates phosphorylation of at least one receptor tyrosine kinase, and wherein the antibody substance inhibits the first and second growth factors to which it binds from stimulating phosphorylation of the receptor tyrosine kinases. In a further embodiment, the invention provides a polynucleotide comprising a nucleotide sequence that encodes an antibody substance produced by the processes described herein.

To provide a further example, the invention is an isolated polynucleotide comprising a nucleotide sequence that encodes an antibody substance which comprises an antibody variable region of an antibody that binds the first growth factor and an antibody variable region of an antibody that binds the second growth factor in which the antibody variable regions are attached to one another.

To provide another example, the isolated polynucleotide encodes an antibody substance in which antibody heavy and light chain variable regions that binds a first growth factor is attached to an antibody heavy and light chain variable regions that binds a second growth factor. In a further embodiment, the antibody heavy and light chain variable regions of the first antibody are attached to each other to form a single polypeptide and the antibody heavy and light chain variable regions of the second antibody are attached to each other to form a single polypeptide.

Another aspect of the invention is an expression vector comprising an isolated polynucleotide of the invention. The expression vector may be any expression vector suitable for transfection or transformation into, and expression of proteins in either prokaryotic or eukaryotic host cells. It is contemplated that the expression vector comprises an expression control sequence operably linked to a polynucleotide of the invention.

Vectors are useful for expressing antibody substances in a variety of host cell systems, including but not limited to bacterial (e.g., E. Coli, Bacillus, Salmonella), yeast (Saccharomyces), insect, mammalian, and human cell lines. Gene therapy vectors also are useful for effecting expression in vivo. The term “vector” refers to a nucleic acid molecule amplification, replication, and/or expression vehicle, often derived from or in the form of a plasmid or viral DNA or RNA system, where the plasmid or viral DNA or RNA may be functional in a selected host cell. The vector may remain independent of host cell genomic DNA or may integrate in whole or in part with the genomic DNA. Preferred vectors contain all necessary elements so as to be functional in a selected host cell. Nucleic acid encoding an antibody polypeptide of interest is inserted into an amplification and/or expression vector to increase the copy number of the gene and/or to express the encoded polypeptide in a suitable host cell and/or to transform cells in a target organism (to express the polypeptide in vivo). Selection of the host cell will depend at least in part on whether the polypeptide or fragment thereof is to be glycosylated. If so, mammalian and preferably human host cells are preferable.

Vectors typically contain 5′ flanking sequence and other regulatory elements such as an enhancer(s), a promoter, an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Optionally, the vector may contain a “tag” sequence, i.e., an oligonucleotide sequence located at the 5′ or 3′ end of the coding sequence that encodes polyHis (such as hexaHis) or another small immunogenic sequence. This tag will be expressed along with the protein, and can serve as an affinity tag for purification of the polypeptide from the host cell. Optionally, the tag can subsequently be removed from the purified polypeptide by various means such as using a selected peptidase. A transcription termination element is typically located 3′ to the end of the polypeptide coding sequence and serves to terminate transcription of the polypeptide. All of the elements set forth above, as well as others useful in this invention, are well known to the skilled artisan and are described, for example, in Sambrook, et al., “Molecular Cloning: A Laboratory Manual,” Cold Spring Harbor Laboratory Press, Cold Spring Harbor, N.Y. (1989) and Berger, et al., eds., “Guide To Molecular Cloning Techniques,” Academic Press, Inc., San Diego, Calif. (1987).

Numerous vectors are commercially available, including bacterial vectors pHE4 (ATCC Accession Number 209645), pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRlT5 available from Pharmacia. Eukaryotic vectors include pWL EO, pSV2CAT, pOG44, pXT1 and pSG available from Stratagene; and pSVK3, pBPV, pMSG and pSVL available from Pharmacia. Yeast expression vectors include pYES2, pYD1, pTEF1/Zeo, pYES2/GS, pPICZ, pGAPZ, pGAPZalpha, pPIC9, pPIC3.5, pHIL-D2, pHIL-S1, pPIC3:5K, pPIC9K, and PA0815 (all available from Invitrogen, Carlsbad, Calif.).

DNA may be introduced into a cell using a variety of viral vectors. In such embodiments, expression constructs comprising viral vectors containing the genes of interest may be adenoviral (see, for example, U.S. Pat. No. 5,824,544; U.S. Pat. No. 5,707,618; U.S. Pat. No. 5,693,509; U.S. Pat. No. 5,670,488; U.S. Pat. No. 5,585,362, each incorporated herein by reference), retroviral (see, for example, U.S. Pat. No. 5,888,502; U.S. Pat. No. 5,830,725; U.S. Pat. No. 5,770,414; U.S. Pat. No. 5,686,278; U.S. Pat. No. 4,861,719, each incorporated herein by reference), adeno-associated viral (see, for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479, each incorporated herein by reference), an adenoviral-adenoassociated viral hybrid (see, for example, U.S. Pat. No. 5,856,152 incorporated herein by reference) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688, each incorporated herein by reference) vector.

The invention further provides a host cell transformed or transfected with a polynucleotide that comprises a nucleotide sequence encoding an antibody substance, antibody or polypeptide contemplated by the invention. In a further aspect, the invention provides a host cell transformed or transfected with the expression vector encoding an antibody substance, antibody or polypeptide contemplated by the invention, wherein the cell expresses the antibody substance, antibody, or polypeptide encoded by the polynucleotide. The host cell of the invention may be any host cell suitable for expression of mammalian proteins. The host cell may be prokaryotic (e.g., bacterial, such as E. coli) or eukaryotic (e.g,. yeast, plant, mammalian, or human). In a preferred embodiment, the host cell is a mammalian host cell.

The host cells containing the vector (i.e., transformed or transfected) may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of the transformed cells only is added as a supplement to the media.

In a related aspect the invention contemplates a method for producing an antibody substance, antibody, or polypeptide contemplated by the invention, comprising culturing a host cell transfected with an expression vector as contemplated by the invention in a culture medium, and recovering the antibody substance, antibody, or polypeptide from the cell or the medium.

Every method of using antibody substances of the invention, whether for therapeutic, diagnostic, or research purposes, is another aspect of the invention.

For example, the invention further contemplates use of the antibody substances as a method for screening for inhibition of growth factor binding to receptor and decrease in receptor activation. In one aspect the invention provides a method of screening an antibody substance for growth factor neutralization activity comprising: contacting a growth factor and a growth factor receptor in the presence and absence of an antibody substance; and measuring binding between the growth factor and the growth factor receptor in the presence and absence of the antibody substance, wherein reduced binding in the presence of the antibody substance indicates growth factor neutralization activity for the antibody substance; wherein the growth factor comprises at least one member selected from the group consisting of VEGF-A, VEGF-B, VEGF-C, VEGF-D, VEGF-E, PlGF, PDGF-A, PDGF-B, PDGF-C, and PDGF-D; and combinations thereof; wherein the receptor is at least one member selected from the group consisting of VEGFR-1, VEGFR-2, VEGFR-3, PDGFR-α, PDGFR-β; an extracellular domain fragment of any of said receptors that is effective to bind to the growth factor; a chimeric receptor comprising the extracellular domain fragment; and combinations thereof; and wherein the antibody substance comprises an antibody substance according to the invention.

It is further contemplated in the screening method that the contacting is performed in a cell free system and the measuring of the binding comprises: measuring growth factor bound to the growth factor receptor. In a related embodiment, the contacting comprises contacting a cell that expresses the receptor with the growth factor; and wherein the measuring comprises: measuring growth factor receptor phosphorylation, wherein the phosphorylation is indicative of binding; measuring a growth factor-mediated cellular response in the cell, wherein the cellular response is indicative of binding between the growth factor and the receptor.

The substances are useful as a therapeutic, diagnostic, or research tool for any disorder where one PDGF/VEGF family member is over expressed and especially useful if two or more are overexpressed. For example, the invention includes a method of inhibiting fibrosis comprising administering to a mammalian subject in need of inhibition of fibrosis an antibody substance of the invention, wherein the antibody substance is specific for at least two PDGF molecules (PDGF-A, PDGF-B, PDGF-C, or PDGF-D), in an amount effective to inhibit fibrosis. In one aspect, the fibrosis may be liver fibrosis, cardiac fibrosis, kidney fibrosis or myelofibrosis. In a preferred embodiment, the antibody substance administered to inhibit fibrosis is bispecific for PDGF-C and PDGF-D.

For example, one aspect of the invention is a method for inhibiting angiogenesis or lymphangiogenesis comprising administering to a mammalian subject in need of inhibition of angiogenesis or lymphangiogenesis an antibody substance according to the invention, in an amount effective to inhibit angiogenesis or lymphangiogenesis. Methods to determine the extent of inhibition of angiogenesis and lymphangiogenesis are described herein.

The invention further contemplates a method for inhibiting angiogenesis or lymphangiogenesis comprising administering to a mammalian subject in need of inhibition of angiogenesis or lymphangiogenesis an antibody substance according to the invention, wherein the subject has a disease characterized by neoplastic cell growth exhibiting angiogenesis or lymphangiogenesis, and the antibody substance is administered in an amount effective to inhibit the neoplastic cell growth. Neoplastic cell growth as used herein refers to multiplication of the cells which is uncontrolled and progressive. Cancers, especially vascularized cancers, are examples of neoplastic cell growth that is treatable using materials and methods of the invention.

It is further contemplated that the method of the invention is used to treat a subject that has a disease characterized by aberrant angiogenesis or lymphangiogenesis, including but not limited to, inflammation (chronic or acute), an infection, an immunological disease, arthritis, rheumatoid arthritis, diabetes, retinopathy, psoriasis, arthopathies, congestive heart failure, plasma leakage, fluid accumulation due to vascular permeability, lymphangioma, and lymphangiectasis.

The antibody substances also may be used to treat or prevent cancer associated disorders such as cancer associated ascites formation.

Using materials and methods of the invention it is possible to design or select more effective antibody substances as therapeutics by evaluating a subject to determine which growth factors and growth factor receptors are being expressed, or overexpressed (relative to healthy tissue or fluids) in a neoplastic disease state and therefore may be contributing to the neoplastic cell growth.

Thus, in one aspect, the invention is a method of inhibiting neoplastic cell growth comprising steps of: (a) diagnosing a mammalian subject with neoplastic cell growth, (b) assaying the neoplastic cell growth for expression of two or more growth factors selected from the group consisting of VEGF-A, VEGF-B, VEGF-C, VEGF-D, VEGF-E, PlGF, PDGF-A, PDGF-B, PDGF-C, and PDGF-D, and (c) administering to the subject an antibody substance according to the invention, wherein the antibody substance binds two or more growth factors identified in step (b) as being expressed in the neoplastic cell growth. In one embodiment, the neoplastic cell growth is a tumor.

The invention further provides a method of inhibiting neoplastic cell growth, comprising steps of: (a) diagnosing a mammalian subject with neoplastic cell growth, (b) assaying the neoplastic cell growth for expression of at least one tyrosine kinase receptor selected from the group consisting of VEGFR-1, VEGFR-2, VEGFR-3, PDGFR-alpha, and PDGFR-beta; and (c) administering to the subject an antibody substance according to the invention, wherein the antibody substance binds to two or more growth factors that bind to at least one receptor tyrosine kinase identified in step (b) as being expressed in the neoplastic cell growth.

In a related aspect, the method includes a step further comprising administering to the subject a treatment selected from the group consisting of a chemotherapeutic agent, a radiotherapeutic agent, or radiation therapy. In one embodiment, the antibody substance is administered in combination with a second agent such as a chemotherapeutic agent; a radiotherapeutic agent, radiation therapy, or a growth factor or cytokine. The chemotherapeutic agent or radiotherapeutic agent may be a member of the class of agents including an anti-metabolite; a DNA-damaging agent; a cytokine or growth factor; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radiosensitizer; and a photosensitizer. Specific examples of these agents are described elsewhere in the application.

It is contemplated that the antibody substance, antibody or polypeptide and the second agent are administered simultaneously, in the same formulation. It is further contemplated that the antibody substance and the second agent are administered at different times. In one embodiment, the antibody substance and the second agent are administered concurrently. In a second embodiment, the antibody substance is administered prior to the second agent. In a third embodiment, the antibody substance is administered subsequent to the second agent.

Generally, compositions of the invention are those that will inhibit tumor cell growth and metastasis by inhibiting angiogenesis and lymphangiogenesis and will act at lower concentrations, thereby permitting use of the compositions in a pharmaceutical composition at lower effective doses. Such compositions are suitable for administration by several routes such as intrathecal, parenteral, topical, intranasal, intravenous, intramuscular, inhalational, or any other clinically acceptable route of administration. Thus, in one embodiment, the invention provides a method of treating a subject, wherein the antibody substance, antibody or polypeptide is administered in an amount effective to inhibit angiogenesis or lymphangiogenesis in the subject. In a further embodiment, the subject is suffering from a condition or disorder resulting from aberrant angiogenesis or lymphangiogenesis.

The subject treated by the methods of the invention may be human, or any non-human animal model for human medical research, or an animal of importance as livestock or pets (e.g., companion animals). In one variation, the subject has a disease or condition characterized by a need for modulation of angiogenesis or lymphangiogenesis, and administration of a composition comprising an antibody substance of the invention, antibody or polypeptide improves the animal's state, for example, by palliating disease symptoms, reducing unwanted angiogenesis or lymphangiogenesis, reducing tumor cell survival, or otherwise improving clinical symptoms. In a preferred embodiment, the subject to be treated is human.

As yet another aspect, the invention includes methods of imaging using antibody substance of the invention. The antibody substances can be used to image the quantity and/or distribution of the antigens to which they bind in a biological sample, such as a tissue sample (e.g., a biopsy), or they can be used to image tissues and fluids in vivo. For example, the method comprises contacting a biological sample with, or administering to a mammalian subject, a composition comprising an antibody substance of the invention, and detecting the quantity and or distribution of the antibody bound to the sample or bound to tissues or fluids in the subject. For in vitro imaging, preferred embodiments include a washing step to remove unbound antibody substance. For both in vivo and in vitro applications, a labeled antibody substance is preferred. The quantity or distribution of antibody substance in the tissue sample or the mammalian subject has diagnostic indications. For example, high concentrations may indicate sites of angiogenesis, which may be an indication of prior injury, healing, or neoplastic cell growth; or may indicate fibrosis.

Various PDGF/VEGF family members have growth and differentiation effects on populations of progenitor cells, e.g., stem cells, endothelial progenitor cells, hematopoietic progenitor cells, and the like. Antibody substances of the invention may be used to contact such cells in vitro or in vivo to modulate growth or differentiation of these cells. Antibody substances of the invention may be administered to bind such growth factors for the purpose of modulating the proliferation and/or differentiation of the progenitor cells.

The invention described herein may be used with and recombined with binding construct materials and methods for sequestering PDGF/VEGR polypeptides described in commonly owned U.S. Provisional Patent Application No. 60/550,907 (Attorney Docket No. 28967/39700), also filed on Mar. 5; 2004, and related, co-filed International patent application Ser. No. ______ (Attorney Docket No. 28967/39700A), directed to growth factor constructs materials and methods, the entire text of which are incorporated herein by reference in their entirety.

Additional features and variations of the invention will be apparent to those skilled in the art from the entirety of this application, including the detailed description, and all such features are intended as aspects of the invention. It should be understood, however, that the detailed description and the specific examples, while indicating preferred embodiments of the invention, are given by way of illustration only, because various changes and modifications within the spirit and scope of the invention will become apparent to those skilled in the art from this detailed description.

Moreover, features of the invention described herein can be re-combined into additional embodiments that also are intended as aspects of the invention, irrespective of whether the combination of features is specifically mentioned above as an aspect or embodiment of the invention. By way of example, an embodiment or variation described with respect to one antibody substance of the invention should be understood to apply to other antibody substances of the invention. By way of another example, where uses of antibody substances are described (e.g., therapeutic uses) it should be understood that analogous uses of polynucleotides and vectors that encode the antibody substances also are contemplated. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 depicts an amino acid sequence alignment of human PDGF-C [residues 231-274-(SEQ ID NO: 27), Li et al., Nat. Cell Biol. 2:302-309, 2000] and human PDGF-D [residues 255-296 (SEQ ID NO: 28), Bergsten et al., Nat Cell Biol. 3:512-516, 2001]. Positions with identical amino acids appear in bold, while chemically similar amino acids are denoted by a box. The underlined regions in both proteins are highly likely to be epitopes for crossreacting antibodies.

### DETAILED DESCRIPTION

The present invention addresses a need in the art to develop more therapeutics to slow or halt the spread of tumors by reducing their ability to vascularize. The present invention provides molecules or agents that interact with multiple VEGF/PDGF growth factors to eliminate signaling through their receptors. Abolishing angiogenic signals through VEGFR/PDGFR in and around tumors reduces the tumor's ability to vascularize, grow and metastasize.

In order that the invention may be more completely understood, several definitions are set forth.

The term “derivative” when used in connection with antibody substances and polypeptides of the invention refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins. Derivatives retain the binding properties of underivatized molecules of the invention.

“Detectable moiety” or a “label” refers to a composition detectable by spectroscopic, photochemical, biochemical, immunochemical, or chemical means. For example, useful labels include ³²P, ³⁵S fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantitate the amount of bound detectable moiety in a sample.

“Heavy chain variable region” as used herein refers to the region of the antibody molecule comprising at least one complementarity determining region (CDR) of said antibody heavy chain variable domain. The heavy chain variable region may contain one, two, or three CDR of said antibody heavy chain.

“Light chain variable region” as used herein refers to the region of an antibody molecule, comprising at least one complementarity determining region (CDR) of said antibody light chain variable domain. The light chain variable region may contain one, two, or three CDR of said antibody light chain, which may be either a kappa or lambda light chain depending on the antibody.

As used herein, “potentiate” refers to activity of the bispecific antibody, which, when administered in conjunction with a second agent, such as a chemotherapeutic agent, a radiotherapeutic agent, or a cytokine of growth factor, inhibits of tumor growth and metastasis beyond that of administration the second agent alone, or inhibits equally but with reduced side effects.

The term “prodrug” as used herein refers to compounds that are rapidly transformed in vivo to a more pharmacologically active compound.

The term “specific for,” when used to describe antibodies of the invention, indicates that the variable regions of the antibodies of the invention recognize and bind the polypeptide with a detectable preference (i.e., able to distinguish the polypeptide of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well: known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

A “therapeutically effective amount” or “effective amount” refers to that amount of the compound sufficient to result in amelioration of symptoms, for example, treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Antibody Variant” as used herein refers to a bispecific antibody polypeptide sequence that contains at least one amino acid substitution, deletion, or insertion in the variable region of the natural antibody variable region domains. Variants may be substantially homologous or substantially identical to the unmodified antibody.

PDGF/VEGF Family Members

The VEGF subfamily is composed of PDGF/VEGF members which share a VEGF homology domain (VHD) characterized by the sequence: C-X(22-24)-P-[PSR]-C-V-X(3)-R-C-[GSTA]-G-C-C-X(6)-C-X(32-41)-C.

VEGF-A (SEQ ID NOs: 1 and 2) is a secreted, disulfide-linked homodimeric glycoprotein composed of 23 kD subunits. Five human VEGF-A isoforms of 121, 145, 165, 189 or 206 amino acids in length (VEGF₁₂₁₋₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparan-sulfate proteoglycans, which behave as low affinity receptors for VEGF-A. VEGF₁₂₁ does not bind to either heparin or heparan-sulfate; VEGF₁₄₅ and VEGF₁₆₅ (GenBank Acc. No. M32977) are both capable of binding to heparin; and VEGF₁₈₉ and VEGF₂₀₆ show the strongest affinity for heparin and heparan-sulfates. VEGF₁₂₁, VEGF₁₄₅, and VEGF₁₆₅ are secreted in a soluble form, although most of VEGF₁₆₅ is confined to cell surface and extracellular matrix proteoglycans, whereas VEGF₁₈₉ and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF₁₂₁ and VEGF₁₆₅ are the predominant forms, whereas VEGF₂₀₆ is rarely detected (Ferrara, J Mol Med 77:527-543, 1999). VEGF₁₄₅ differs in that it is primarily expressed in cells derived from reproductive organs (Neufeld et al., FASEB J 13:9-22, 1999). Antibodies that are specific for VEGF-A bind at least the soluble secreted forms of VEGF-A, and preferably also bind cell surface-associated forms.

PlGF (SEQ ID NOs: 3 and 4), a second member of the VEGF subfamily, is generally a poor stimulator of angiogenesis and endothelial cell proliferation in comparison to VEGF-A, and the in vivo role of PlGF is not well understood. Three isoforms of PlGF produced by alternative mRNA splicing have been described (Hauser et al., Growth Factors 9:259-268, 1993; Maglione et al., Oncogene 8:925-931, 1993). PlGF forms both disulfide-linked homodimers and heterodimers with VEGF-A. The PlGF-VEGF-A heterodimers are more effective at inducing endothelial cell proliferation and angiogenesis than PlGF homodimers. PlGF is primarily expressed in the placenta, and is also co-expressed with VEGF-A during early embryogenesis in the trophoblastic giant cells of the parietal yolk sac (Stacker and Achen, Growth Factors 17:1-11, 1999).

VEGF-B (SEQ ID NOs: 5 and 6), described in detail in International Patent Publication No. WO 96/26736 and U.S. Pat. Nos. 5,840,693 and 5,607,918, incorporated herein by reference, shares approximately 44% amino acid identity with, VEGF-A. Although the biological functions of VEGF-B in vivo remain incompletely understood, it has been shown to have angiogenic properties, and may also be involved in cell adhesion and migration, and in regulating the degradation of extracellular matrix. VEGF-B is expressed as two isoforms of 167 and 186 amino acid residues generated by alternative splicing. VEGF-B₁₆₇ is associated with the cell surface or extracellular matrix via a heparin-binding domain, whereas VEGF-B₁₈₆ is secreted. Both VEGF-B₁₆₇ and VEGF-B₁₈₆ can form disulfide-linked homodimers or heterodimers with VEGF-A. The association to the cell surface of VEGF₁₆₅-VEGF-₁₆₇ heterodimers appears to be determined by the VEGF-B component, suggesting that heterodimerization may be important for sequestering VEGF-A. VEGF-B is expressed primarily in embryonic and adult cardiac and skeletal muscle tissues (Joukov et al., J Cell Physiol 173:211-215, 1997; Stacker and Achen, (supra). Mice lacking VEGF-B survive but have smaller hearts, dysfunctional coronary vasculature, and exhibit impaired recovery from cardiac ischemia (Bellomo et al., Circ Res E29-E35, 2000). Antibodies that are specific for VEGF-B bind at least the circulating VEGF-B₁₈₆ form, and preferably also bind VEGF-B₁₆₇.

VEGF-C (SEQ ID NOs: 7 and 8) is originally expressed as a larger precursor protein, prepro-VEGF-C, having extensive amino- and carboxy-terminal peptide sequences flanking a VEGF homology domain (VHD), with the C-terminal peptide containing tandemly repeated cysteine residues in a motif typical of Balbiani ring 3 protein. The prepro-VEGF-C polypeptide is processed in multiple stages to produce a mature and most active VEGF-C polypeptide (ΔNΔC VEGF-C) of about 21-23 kD (as assessed by SDS-PAGE under reducing conditions). Such processing includes cleavage of a signal peptide (SEQ ID NO: 2, residues 1-31); cleavage of a carboxyl-terminal peptide (corresponding approximately to amino acids 228-419 of SEQ ID NO: 2 to produce a partially-processed form of about 29 kD; and cleavage (apparently extracellularly) of an amino-terminal peptide (corresponding approximately to amino acids 32-102 of SEQ ID NO: 2) to produced a fully-processed mature form of about 21-23 kD. Experimental evidence demonstrates that partially-processed forms of VEGF-C (e.g., the 29 kD form) are able to bind the Flt4 (VEGFR-3) receptor, whereas high affinity binding to VEGFR-2 occurs only with the fully processed forms of VEGF-C. Moreover, it has been demonstrated that amino acids 103-227 of SEQ ID NO: 2 are not all critical for maintaining VEGF-C functions. A polypeptide consisting of amino acids 112-215 (and lacking residues 103-111 and 216-227) of SEQ ID NO: 2 retains the ability to bind and stimulate VEGF-C receptors, and it is expected that a polypeptide spanning from about residue 131 to about residue 211 will retain VEGF-C biological activity. The cysteine residue at position 156 has been shown to be important for VEGFR-2 binding ability. It appears that VEGF-C polypeptides naturally associate as non-disulfide linked dimers. For this invention, antibody substances specific for VEGF-C are substances that bind fully processed forms that lack the amino- and carboxy-terminal polypeptides. Preferred antibodies also bind the partly processed forms that retain the N-terminal polypeptide.

Like VEGF-C, VEGF-D (SEQ ID NOs: 9 and 10) is initially expressed as a prepro-peptide that undergoes removal of a signal peptide (residues 1-21 of SEQ ID NO: 10) N-terminal (residues 22-92 of SEQ ID NO: 10) and C-terminal (residues 202-354 of SEQ ID NO: 10) proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D (SEQ ID NO: 10) and binds VEGFR-2 and VEGFR-3. Partly processed forms of VEGF-D bind to VEGFR-3. For this invention, antibodies specific for VEGF-D bind to fully processed forms of VEGF-D. Preferably, such antibodies also bind to partly processed forms. Monoclonal antibody 4E10, which was generated against the processed form of VEGF-D, is described in U.S. Pat. No. 6,383,484 (Achen et al.). Ligand binding assays performed with the 4E10 antibody demonstrated that 4E10 binds to both VEGF-D and VEGF-C. However, the 4E10 antibody is a non-neutralizing antibody that does not prevent binding of the growth factors to the VEGFR-2 or VEGFR-3 receptors. Thus, while the 4E10 antibody may bind both VEGF-D and VEGF-C and act bispecifically, there still remains a need in the art to identify bispecific antibodies that bind growth factors and neutralize their biological activity.

Preferred VEGF-D antibody substances included those described in co-filed U.S. Provisional Application No. 60/550,441 (Attorney Docket No. 28967/39969), filed Mar. 5, 2004, and related, co-filed International patent application Ser. No. ______ (Attorney Docket No. 28967/39969A), both directed to anti-VEGF-D antibodies and chimeric anti-VEGF-D antibodies and methods of using same, both incorporated herein by reference.

PDGF-A (SEQ ID NOs: 17 and 18) and PDGF-B (SEQ ID NOs: 19 and 20) can homodimerize or heterodimerize to produce three different isoforms: PDGF-AA, PDGF-AB, or PDGF-BB. PDGF-A is only able to bind the PDGF α-receptor (PDGFR-α including PDGFR-α/α homodimers). PDGF-B can bind both the PDGFR-αand a second PDGF receptor (PDGFR-β). More specifically, PDGF-B can bind to PDGFR-α/α and PDGFR-β/β homodimers, as well as PDGFR-α/β heterodimers.

PDGF-AA and -BB are the major mitogens and chemoattractants for cells of mesenchymal origin, but have no, or little effect on cells of endothelial lineage, although both PDGFR-α and -β are expressed on endothelial cells (EC). PDGF-BB and PDGF-AB have been shown to be involved in the stabilization/maturation of newly formed vessels (Isner et al., Nature 415:234-9, 2002; Vale et al., J Interv Cardiol 14:511-28, 2001); Heldin et al., Physiol Rev 79:1283-1316, 1999; Betsholtz et al., Bioessays 23:494-507, 2001). Other data however, showed that PDGF-BB and PDGF-AA inhibited bFGF-induced angiogenesis in vivo via PDGFR-α signaling. PDGF-AA is among the most potent stimuli of mesenchymal cell migration, but it either does not stimulate or it minimally stimulates EC migration. In certain conditions, PDGF-AA even inhibits EC migration (Thommen et al., J Cell Biochem. 64:403-13, 1997; De Marchis et al., Blood 99:2045-53, 2002; Cao et al., FASEB. J 16:1575-83, 2002). Moreover, PDGFR-α has been shown to antagonize the PDGFR-β-induced SMC migration Yu et al. (Biochem. Biophys. Res. Commun. 282:697-700, 2001) and neutralizing antibodies against PDGF-AA enhance smooth muscle cell (SMC) migration (Palumbo, R., et al., Arterioscler. Thromb. Vasc. Biol. 22:405-11, 2002). Thus, the angiogenic/arteriogenic activity of PDGF-A and -B, especially when signaling through PDGFR-α, has been controversial and enigmatic.

PDGF-AA and -BB have been reported to play important roles in the proliferation and differentiation of both cardiovascular and neural stem/progenitor cells. PDGF-BB induced differentiation of Flk1+embryonic stem cells into vascular mural cells (Carmeliet, P., Nature 408:43-45, 2000; Yamashita et al., Nature 408:92-6, 2000), and potently increased neurosphere derived neuron survival (Caldwell et al., Nat Biotechnol. 19:475-479, 2001); while PDGF-AA stimulated oligodendrocyte precursor proliferation through αᵥβ₃ integrins (Baron, et al., Embo. J 21:1957-66, 2002).

The nucleotide and amino acid sequence for PDGF-C are set out in SEQ ID NOs: 11 and 12, respectively, and the nucleotide and amino acid for PDGF-D are set out in SEQ ID NOs: 13 and 14, respectively. PDGF-C binds PDGFR-α/α homodimers and PDGF-D binds PDGFR-β/β homodimers and both have been reported to bind PDGFR-α/β heterodimers. PDGF-C polypeptides and polynucleotides were characterized by Eriksson et al. in International Patent Publication No. WO 00/18212, U.S. Patent Application Publication No. 2002/0164687 A1, and U.S. patent application Ser. No. 10/303,997 [published as U.S. Pat. Publ. No. 2003/0211994]. PDGF-D polynucleotides and polypeptides were characterized by Eriksson, et al. in International Patent Publication No. WO 00/27879 and U.S. Patent Application Publication No. 2002/0164710 A1. These documents are all incorporated by reference in their entirety. As described therein, PDGF-C and -D bind to PDGF receptors alpha and beta, respectively. However, a noteworthy distinction between these polypeptides and PDGF-A and -B is that PDGF-C and -D each possess an amino-terminal CUB domain that can be proteolytically cleaved to yield a biologically active (receptor binding) carboxy-terminal domain with sequence homology to other PDGF family members. Antibodies of this invention that are specific for PDGF-C or -D bind biologically active forms lacking the CUB domain. Preferred antibodies also bind unprocessed forms.

Still another class of preferred antibodies are antibodies that specifically bind one of the PDGF/VEGF growth factor family members and inhibit the family member from forming heterodimers with other family members that have been shown to heterodimerize with the family member. Such heterodimers form between VEGF-A and -B, and between VEGF-B and PlGF, for example, and antibody substances specific for any of these family members that prevent the heterodimerization are preferred.

During development, PDGF-C is expressed in muscle progenitor cells and differentiated smooth muscle cells in most organs, including the heart, lung and kidney (Aase et al., Mech. Dev. 110:187-91, 2002). In adulthood, PDGF-C is widely expressed in most organs, with the highest expression level in the heart and kidney (Li et al., Nat. Cell. Biol. 2:302-09, 2000). PDGF-CC is secreted as an inactive homodimer of approximately 95 kD. Upon proteolytic removal of the CUB domain, PDGF-CC is capable of binding and activating its receptor, PDGFR-α (Li et al., Cytokine & Growth Factor Reviews 244:1-8, 2003). In cells co-expressing both PDGFR-α and -β, PDGF-CC may also activate the PDGFR-α/β heterodimer, but not the PDGFR-β/β homodimer (Cao et al., FASEB. J. 16:1575-83, 2002; Gilbertson et al., J. Biol. Chem. 276:27406-14, 2001).

Active PDGF-CC is a potent mitogen for fibroblast and vascular smooth muscle cells (Li et al., Nat. Cell. Biol. 2:302-09, 2000; Cao, et al., FASEB. J 16:1575-83, 2002; Uutela et al., Circulation 103:2242-7, 2001). Both PDGF-AA and PDGF-CC bind PDGFR-α, but only PDGF-CC potently stimulates angiogenesis in mouse cornea pocket and chick chorioallanoic membrane (CAM) assays (Cao, et al., FASEB. J. 16:1575-83, 2002). PDGF-CC also promotes wound healing by stimulating tissue vascularization (Gilbertson et al., supra). However, these studies did not address whether PDGF-CC stimulated vessel growth by affecting endothelial or smooth muscle cells, nor did they examine whether PDGF-CC promoted the maturation of newly formed vessels (including vasculogenesis, angiogenesis, neoangiogenesis and arteriogenesis).

Four additional members of the VEGF subfamily collectively referred to as VEGF-E factors have been identified in poxviruses, which infect humans, sheep and goats. The orf virus-encoded VEGF-E (SEQ ID NOs: 15 and 16) and NZ2 VEGF are potent mitogens and permeability enhancing factors. Both show approximately 25% amino acid identity to mammalian VEGF-A, and are expressed as disulfide-linked homodimers. Another variant of orf virus VEGF-E like protein from strain NZ10 is described in WO 00/25805, incorporated here by reference. Infection by these viruses is characterized by pustular dermititis which may involve endothelial cell proliferation and vascular permeability induced by these viral VEGF proteins (Ferrara, J Mol Med 77:527-543, 1999; Stacker and Achen, Growth Factors 17:1-11, 1999). VEGF-like proteins have also been identified from two additional strains of the orf virus, D1701 (GenBank Acc. No. AF106020; described in Meyer et al., EMBO J. 18:363-374, 1999) and NZ10 [described in International Patent Application WO 00/25805 (incorporated herein by reference) the sequence of which is set out in SEQ ID NO: 21 and 22]. These viral VEGF-like proteins have been shown to bind VEGFR-2 present on host endothelium, and this binding is important for development of infection and viral induction of angiogenesis (Meyer et al., EMBO J. 18:363-374, 1999; International Patent Application WO 00/25805).

PDGF/VEGF Receptors

Seven cell surface receptors that interact with PDGF/VEGF family members have been identified. These include PDGFR-α (see e.g., GenBank Acc. No. NM006206), PDGFR-β (see e.g., GenBank Acc. No. NM002609), VEGFR-1/Flt-1 (fins-like tyrosine kinase-1; GenBank Acc. No. X51602; De Vries et al., Science 255:989-991 (1992)); VEGFR-2/KDR/Flk-1 (kinase insert domain containing receptor/fetal liver kinase-1; GenBank Acc. Nos. X59397 (Flk-1) and L04947 (KDR); (Terman et al., Biochem Biophys Res Comm 187:1579-1586, 1992; Matthews et al., Proc Natl Acad Sci USA 88:9026-9030, 1991); VEGFR-3/Flt4 (fins-like tyrosine kinase 4; U.S. Pat. No. 5,776,755 and GenBank Acc. No. X68203 and S66407; Pajusola et al., Oncogene 9:3545-3555, 1994; neuropilin-1 (Gen Bank Acc. No. NM003873), and neuropilin-2 (Gen Bank Acc. No. NM003872).

The two PDGF receptors mediate signaling of PDGFs as described above. VEGF₁₂₁, VEGF₁₆₅, VEGF-B, PlGF-1 and PlGF-2 bind VEGFR-1; VEGF₁₂₁, VEGF₁₄₅, VEGF₁₆₅, VEGF-C, VEGF-D, VEGF-E, and NZ2 VEGF bind VEGFR-2; VEGF-C and VEGF-D bind VEGFR-3; VEGF₁₆₅, VEGF-B, PlGF-2, VEGF-C, and NZ2 VEGF bind neuropilin-1; and VEGF₁₆₅, VEGF₁₄₅ and VEGF-C bind neuropilin-2. (Neufeld et al., FASEB J 13:9-22, 1999; Stacker and Achen, Growth Factors 17:1-11, 1999; Ortega et al., Fron Biosci 4:141-152, 1999; Zachary, Intl J Biochem Cell Bio 30:1169-1174, 1998; Petrova et al., Exp Cell Res 253:117-130, 1999; Gluzman-Poltorak et al., J. Biol. Chem. 275:18040-45, 2000, U.S. Patent Publ. No. 2003/0113324). A ligand for Tek/Tie-2 has been described (International Patent Application No. PCT/US95/12935 (WO 96/11269) by Regeneron Pharmaceuticals, Inc.); however, the ligand for Tie has not yet been identified.

The PDGFR is found as either a homodimer or heterodimer of the subunits PDGFRα and PDGFRβ. PDGF-A is only able to bind the PDGF α-receptor including PDGFR-α/α homodimers. PDGF-B can bind to PDGFR-α/α and PDGFR-β/β homodimers, as well as PDGFR-α/β heterodimers. PDGFR-α/α homodimers bind PDGF-C and PDGFR-β/β homodimers bind PDGF-D, and both PDGF-C and -D have been reported to bind PDGFR-α/β heterodimers.

Several of the VEGF receptors are expressed as more than one isoform. A soluble isoform of VEGFR-1 lacking the seventh Ig-like loop, transmembrane domain, and the cytoplasmic region is expressed in human umbilical vein endothelial cells. This VEGFR-1 isoform binds VEGF-A with high affinity and is capable of preventing VEGF-A-induced mitogenic responses (Ferrara, J Mol Med 77:527-543, 1999; Zachary, Intl J Biochem Cell Bio 30:1169-1174, 1998). A C-terminal truncated from of VEGFR-2 has also been reported (Zachary, supra). In humans, there are two isoforms of the VEGFR-3 protein which differ in the length of their C-terminal ends. Studies suggest that the longer isoform is responsible for most of the biological properties of VEGFR-3.

The expression of VEGFR-1 occurs mainly in vascular endothelial cells, although some may be present on monocytes, trophoblast cells, and renal mesangial cells (Neufeld et al., FASEB J 13:9-22, 1999). High levels of VEGFR-1 mRNA are also detected in adult organs, suggesting that VEGFR-1 has a function in quiescent endothelium of mature vessels not related to cell growth. VEGFR-1−/− mice die in utero between day 8.5 and 9.5. Although endothelial cells developed in these animals, the formation of functional blood vessels was severely impaired, suggesting that VEGFR-1 may be involved in cell-cell or cell-matrix interactions associated with cell migration. Recently, it has been demonstrated that mice expressing a mutated VEGFR-1 in which only the tyrosine kinase domain was missing show normal angiogenesis and survival, suggesting that the signaling capability of VEGFR-1 is not essential (Neufeld et al., supra; Ferrara, supra).

VEGFR-2 expression is similar to that of VEGFR-1 in that it is broadly expressed in the vascular endothelium, but it is also present in hematopoietic stem cells, megakaryocytes, and retinal progenitor cells (Neufeld et al., supra). Although the expression pattern of VEGFR-1 and VEGFR-2 overlap extensively, evidence suggests that, in most cell types, VEGFR-2 is the major receptor through which most of the VEGFs exert their biological activities. Examination of mouse embryos deficient in VEGFR-2 further indicate that this receptor is required for both endothelial cell differentiation and the development of hematopoietic cells (Joukov et al., J Cell Physiol 173:211-215, 1997).

VEGFR-3 is widely expressed on endothelial cells during early embryonic development but as embryogenesis proceeds becomes restricted to venous endothelium and then to the lymphatic endothelium (Kaipainen et al., Cancer Res., 54: 6571-6577, 1994; Kaipainen et al., Proc. Natl. Acad. Sci. USA, 92: 3566-3570, 1995). In adults, the lymphatic endothelia and some high endothelial venules express VEGFR-3, and increased expression occurs in lymphatic sinuses in metastatic lymph nodes and in lymphangioma. VEGFR-3 is also expressed in a subset of CD34⁺ hematopoietic cells which may mediate the myelopoietic activity of VEGF-C demonstrated by overexpression studies (WO 98/33917). Targeted disruption of the VEGFR-3 gene in mouse embryos leads to failure of the remodeling of the primary vascular network, and death after embryonic day 9.5 (Dumont et al., Science 282:946-949, 1998).

VEGFR-3 receptor is essential for vascular development during embryogenesis. Abnormal development or function of the lymphatic endothelial cells can result in tumors or malformations of the lymphatic vessels, such as lymphangiomas or lymphangiectasis. Witte, et al., Regulation of Angiogenesis (eds. Goldber, I. D. & Rosen, E. M.) 65-112 (Birkäuser, Basel, Switzerland, 1997). The VEGFR-3 receptor is upregulated in many types of vascular tumors, including Kaposi's sarcomas (Jussila, et al., Cancer Res 58, 1955-1604, 1998); Partanen, et al., Cancer 86:2406-2412, 1999). The importance of VEGFR-3 signaling for lymphangiogenesis was revealed in the genetics of familial lymphedema, a disease characterized by a hypoplasia of cutaneous lymphatic vessels, which leads to a disfiguring and disabling swelling of the extremities (Witte, et al., supra; Rockson, S. G., Am. J. Med. 110, 288-295, 2001). These studies suggest an essential role for VEGFR-3 in the development of the embryonic vasculature, and also during lymphangiogenesis.

Structural analyses of the VEGF receptors indicate that the VEGF-A binding site on VEGFR-1 and VEGFR-2 is located in the second and third Ig-like loops. Similarly, the VEGF-C and VEGF-D binding sites on VEGFR-2 and VEGFR-3 are also contained within the second Ig-loop (Taipale et al., Curr Top Microbiol Immunol 237:85-96, 1999). The second Ig-like loop also confers ligand specificity as shown by domain swapping experiments (Ferrara, J Mol Med 77:527-543, 1999). Receptor-ligand studies indicate that dimers formed by the VEGF family proteins are capable of binding two VEGF receptor molecules, thereby dimerizing VEGF receptors. The fourth Ig-like loop on VEGFR-1, and also possibly on VEGFR-2, acts as the receptor dimerization domain that links two receptor molecules upon binding of the receptors to a ligand dimer (Ferrara, sypra). Although the regions of VEGF-A that bind VEGFR-1 and VEGFR-2 overlap to a large extent, studies have revealed two separate domains within VEGF-A that interact with either VEGFR-1 or VEGFR-2, as well as specific amino acid residues within these domains that are critical for ligand-receptor interactions. Mutations within either VEGF receptor-specific domain that specifically prevent binding to one particular VEGF receptor have also been recovered (Neufeld et al., supra).

VEGFR-1 and VEGFR-2 are structurally similar, share common ligands (VEGF₁₂₁ and VEGF₁₆₅), and exhibit similar expression patterns during development. However, the signals mediated through VEGFR-1 and VEGFR-2 by the same ligand appear to be slightly different. VEGFR-2 has been shown to undergo autophosphorylation in response to VEGF-A, but phosphorylation of VEGFR-1 under identical conditions was barely detectable. VEGFR-2 mediated signals cause striking changes in the morphology, actin reorganization, and membrane ruffling of porcine aortic endothelial cells recombinantly overexpressing this receptor. In these cells, VEGFR-2 also mediated ligand-induced chemotaxis and mitogenicity; whereas VEGFR-1-transfected cells lacked mitogenic responses to VEGF-A. Mutations in VEGF-A that disrupt binding to VEGFR-2 fail to induce proliferation of endothelial cells, whereas VEGF-A mutants that are deficient in binding VEGFR-1 are still capable of promoting endothelial proliferation. Similarly, VEGF stimulation of cells expressing only VEGFR-2 leads to a mitogenic response whereas comparable stimulation of cells expressing only VEGFR-1 also results in cell migration, but does not induce cell proliferation. In addition, phosphoproteins co-precipitating with VEGFR-1 and VEGFR-2 are distinct, suggesting that different signaling molecules interact with receptor-specific intracellular sequences.

One hypothesis is that the primary function of VEGFR-1 in angiogenesis may be to negatively regulate the activity of VEGF-A by binding it and thus preventing its interaction with VEGFR-2, whereas VEGFR-2 is thought to be the main transducer of VEGF-A signals in endothelial cells. In support of this hypothesis, mice deficient in VEGFR-1 die as embryos while mice expressing a VEGFR-1 receptor capable of binding VEGF-A but lacking the tyrosine kinase domain survive and do not exhibit abnormal embryonic development or angiogenesis. In addition, analyses of VEGF-A mutants that bind only VEGFR-2 show that they retain the ability to induce mitogenic responses in endothelial cells. However, VEGF-mediated migration of monocytes is dependent on VEGFR-1, indicating that signaling through this receptor is important for at least one biological function. In addition, the ability of VEGF-A to prevent the maturation of dendritic cells is also associated with VEGFR-1 signaling, suggesting that VEGFR-1 may function in cell types other than endothelial cells. (Ferrara et al., supra; Zachary et al., supra).

Disruption of the VEGFR genes results in aberrant development of the vasculature leading to embryonic lethality around midgestation. Analysis of embryos carrying a completely inactivated VEGFR-1 gene suggests that this receptor is required for functional organization of the endothelium (Fong et al., Nature, 376: 66-70, 1995). However, deletion of the intracellular tyrosine kinase domain of VEGFR-1 generates viable mice with a normal vasculature (Hiratsuka et al., Proc. Natl. Acad. Sci. USA, 95:9349-9354, 1998). The reasons underlying these differences remain to be explained but suggest that receptor signaling via the tyrosine kinase is not required for the proper function of VEGFR-1. Analysis of homozygous mice with inactivated alleles of VEGFR-2 suggests that this receptor is required for endothelial cell proliferation, hematopoesis and vasculogenesis (Shalaby et al., Nature 376:62-66, 1995; Shalaby et al., Cell 89:981-990, 1997). Inactivation of VEGFR-3 results in cardiovascular failure due to abnormal organization of the large vessels (Dumont et al., Science 282:946-949, 1998).

Of particular interest to the present invention is the fact that one or more of the VEGF/PDGF receptor tyrosine kinases are frequently detected in pathogenic, neoplastic cell growth. For example, the receptors may be expressed on tumor cells themselves, or on blood or lymphatic vessel cells, such as endothelial and smooth muscle cells, that supply blood to neoplastic cells such as tumors, or in the case of lymphatics, may contribute to tumor metastases. Antibody substances of the invention are useful for preventing activation of such receptors by the multiple growth factor ligands that can bind the receptors, thereby directly or indirectly (e.g., by inhibiting angiogenesis or lymphangiogenesis) inhibiting the growth or migration of neoplastic cells.

Antibodies

Bispecific antibodies of the invention are useful for modulating PDGF/VEGF family member mitogenic activity by inhibiting growth factor stimulation of PDGFR/VEGFR signaling. The invention provides antibody substances for administration to human beings (e.g., monoclonal and polyclonal antibodies, single chain antibodies, chimeric antibodies, bifunctional/bispecific antibodies, humanized antibodies, human antibodies, and complementarity determining region (CDR)-grafted antibodies, including compounds which include CDR sequences which specifically recognize a polypeptide of the invention) specific for polypeptides of interest to the invention, especially PDGF/VEGF molecules. Preferred antibodies are human antibodies which are produced and identified according to methods described in WO 93/11236, published Jun. 20, 1993, which is incorporated herein by reference in its entirety. Antibody fragments, including Fab, Fab′, F(ab′)₂, Fv, and single chain antibodies (scFᵥ) are also provided by the invention.

Various procedures known in the art may be used for the production of polyclonal antibodies to PDGF/VEGF molecules or peptide fragments thereof. For the production of antibodies, any suitable host animal (including but not limited to rabbits, mice, rats, or hamsters) are immunized by injection with a PDGF/VEGF protein or peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG (Bacille Calmette-Guerin) and Corynebacterium parvum.

A monoclonal antibody to a PDGF/VEGF protein may be prepared by using any technique which provides for the production of antibody molecules by continuous cell lines in culture. These include but are not limited to the hybridoma technique originally described by Köhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure. Preparation of monoclonal antibodies specific for some PDGF/VEGF molecules has been described. Monoclonal antibodies specific for VEGF-A are described in U.S. Pat. No. 5,730,977 to Ooka et al. Generation of VEGF-B specific monoclonal antibodies is described in U.S. Pat. No. 6,331,301. Antibodies specific for VEGF-C are described in U.S. Pat. No. 6,403,088. Production of VEGF-D monoclonal antibodies is described in U.S. Pat. No. 6,383,484. Monoclonal antibodies to PlGF are described in U.S. Patent Publication No. 2003/0180286.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al., Proc Natl Acad Sci 81: 6851-6855, 1984; Neuberger et al., Nature 312: 604-608, 1984; Takeda et al., Nature 314: 452-454; 1985). Alternatively, techniques described for the production of single-chain antibodies (U.S. Pat. No. 4,946,778) can be adapted to produce PDGF/VEGF-specific single chain antibodies.

Antibody fragments that contain the idiotype of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. (Nature 321: 522-525, 1986), Riechmann et al., (Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2^{nd} Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. For example, using the VEGF-D-specific monoclonal antibody secreted by hybridoma 4A5 (ATCC Deposit No. HB-12698, described in U.S. Pat. No. 6,383,484), polypeptide compositions comprising 4A5-isolated CDRs are generated. Polypeptide compositions comprising one, two, three, four, five and/or six complementarity determining regions of a monoclonal antibody secreted by hybridoma 4A5 are also contemplated. Using the conserved framework sequences surrounding the CDRs, PCR primers complementary to these consensus sequences are generated to amplify the 4A5 CDR sequence located between the primer regions. Techniques for cloning and expressing nucleotide and polypeptide sequences are well-established in the art [see e.g., Sambrook et al., Molecular Cloning: A Laboratory Manual, 2^{nd} Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

It is contemplated that modified polypeptide compositions comprising one, two, three, four, five, and/or six CDRs of a monoclonal antibody are generated, wherein a CDR is altered to provide increased specificity or affinity to the growth factor molecule. Sites within antibody CDRs are typically modified in series, e.g., by substituting first with conservative choices (e.g., hydrophobic amino acid substituted for a non-identical hydrophobic amino acid) and then with more dissimilar choices (e.g., hydrophobic amino acid substituted for a charged amino acid), and then deletions or insertions may be made at the target site. Antibody substances comprising the modified CDRs are screened for binding affinity for the original antigen. Additionally, the anti body or polypeptide is further tested for its ability to neutralize the activity of the target antigens. For example, bispecific antibodies of the invention may be analyzed as set out in the Examples to determine their ability to interfere with growth factor stimulation of receptor phosphorylation.

“Conservative” amino acid substitutions are made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E). “Insertions” or “deletions” are preferably in the range of about 1 to 20 amino acids, more preferably 1 to 10 amino acids. The variation may be introduced by systematically making substitutions of amino acids in a polypeptide molecule using recombinant DNA techniques and assaying the resulting recombinant variants for activity. Nucleic acid alterations can be made at sites that differ in the nucleic acids from different species (variable positions) or in highly conserved regions (constant regions). Methods for expressing polypeptide compositions useful in the invention are described in greater detail below.

Rapid, large-scale recombinant methods for generating antibodies may be employed, such as phage display (Hoogenboom et al., J. Mol. Biol. 227: 381, 1991; Marks et al., J. Mol. Biol. 222: 581, 1991) or ribosome display methods, optionally followed by affinity maturation [see, e.g., Ouwehand et al., Vox Sang 74 (Suppl 2):223-232, 1998; Rader et al., Proc. Natl. Acad. Sci. USA 95:8910-8915, 1998; Dall'Acqua et al., Curr. Opin. Struct. Biol. 8:443-450, 1998]. Phage-display processes mimic immune selection through the display of antibody repertoires on the surface of filamentous bacteriophage, and subsequent selection of phage by their binding to an antigen of choice. One such technique is described in WO 99/10494, which describes the isolation of high affinity and functional agonistic antibodies for MPL and msk receptors using such an approach.

Bispecific Antibodies and Multivalent Antibody Substances

The invention provides for bispecific antibodies in which two different antigen-binding sites are incorporated into a single molecule. Bispecific antibodies are produced, isolated, and tested using standard procedures that have been described in the literature. See, e.g., Pluckthun & Pack, Immunotechnology, 3:83-105 (1997); Carter et al., J Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties.

Bispecific antibodies may be prepared by chemical cross-linking (Brennan et al., Science 229:81, 1985; Raso et al., J. Biol. Chem. 272:27623, 1997), disulfide exchange, production of hybrid-hybridomas (quadromas), by transcription and translation to produce a single polypeptide chain embodying a bispecific antibody, or by transcription and translation to produce more than one polypeptide chain that can associate covalently to produce a bispecific antibody. The contemplated bispecific antibody can also be made entirely by chemical synthesis. The bispecific antibody may comprise two different variable regions, two different constant regions, a variable region and a constant region, or other variations.

“Quadromas”, or “hybrid hybridomas”, may be constructed by fusing hybridomas that secrete two different types of antibodies against two different antigens (Milstein et al., Nature 305:537, 1983; Kurokawa et al., Biotechnology 7:1163, 1989). As used herein, the term “hybrid hybridoma” is used to describe the productive fusion of two B cell hybridomas. Using now standard techniques, two antibody producing hybridomas are fused to give daughter cells, and those cells that have maintained the expression of both sets of clonotype immunoglobulin genes are then selected. Bispecific antibodies can also be prepared by the transfectoma method (Morrison, Science 229:1202, 1985). The invention additionally encompasses bispecific antibody structures produced within recombinant microbial hosts as described in PCT application WO 93/11161 and Holliger, et al., Proc. Natl. Acad. Sci. USA 90:6444, 1993).

To initially select the bispecific antibody, standard methods such as ELISA are used wherein the wells of microtiter plates are coated with a target antigen that specifically interacts with one of the parent hybridoma antibodies and that lacks cross-reactivity with the other parent antibody. Antibodies that demonstrate positive binding to the first agent are then assessed for binding the second target antigen in an ELISA wherein the wells are coated with the second target antigen. Antibodies positive for binding to both antigens are considered bispecific antibodies. In addition, FACS, immunofluorescence staining, idiotype specific antibodies, antigen binding competition assays, and other methods common in the art of antibody characterization may be used in conjunction with the present invention to identify preferred hybrid hybridomas. Once a multivalent antibody has been assessed for binding to all its target antigens, the antibody is further tested for its ability to neutralize the activity of the target antigens. For example, bispecific antibodies of the invention may be analyzed as set out in the Examples to determine their ability to interfere with growth factor stimulation of receptor phosphorylation.

A bispecific antibody can be generated by enzymatic conversion of two different monoclonal antibodies, each comprising two identical L (light chain)-H (heavy chain) half molecules and linked by one or more disulfide bonds, into two F(ab′)₂ molecules, splitting each F(ab′)₂ molecule under reducing conditions into the Fab′ thiols, derivatizing one of these Fab′ molecules of each antibody with a thiol activating agent and combining an activated Fab′ molecule bearing specificity for a first PDGF/VEGF molecule with a non-activated Fab′ molecule bearing specificity for a second PDGF/VEGF molecule, in order to obtain the desired bispecific antibody F(ab′)₂ fragment.

As enzymes suitable for the conversion of an antibody into its F(ab′)₂ or Fab′ molecules, pepsin and papain may be used. In some cases, trypsin or bromelin are suitable. The conversion of the disulfide bonds into the free SH-groups (Fab′ molecules) may be performed by reducing compounds, such as dithiothreitol (DTT), mercaptoethanol, and mercaptoethylamine. Thiol activating agents according to the invention which prevent the recombination of the thiol half-molecules, are 5,5′-dithiobis(2-nitrobenzoic acid) (DTNB), 2,2′-dipyridinedisulfide, 4,4′-dipyridinedisulfide or tetrathionate/sodium sulfite (see also Raso et al., Cancer Res. 42:457, 1982), and references incorporated therein.

The treatment with the thiol-activating agent is generally performed only with one of the two Fab′ fragments. Principally, it makes no difference which one of the two Fab′ molecules is converted into the activated Fab′ fragment (e.g., Fab′-TNB). Generally, however, the Fab′ fragment being more labile is modified with the thiol-activating agent. The conjugation of the activated Fab′ derivative with the free hinge-SH groups of the second Fab′ molecule to generate the bivalent F(ab′)₂ antibody occurs spontaneously at temperatures between 0° and 30° C. The yield of purified F(ab′)₂ antibody is 20-40% (starting from the whole antibodies).

Bispecific molecules of this invention can also be prepared by conjugating a polynucleotide encoding a binding region of a first PDGF/VEGF antibody to a polynucleotide encoding at least the binding region of an antibody chain which recognizes a second PDGF/VEGF molecule. This construct is transfected into a host cell (such as a myeloma) which constitutively expresses the corresponding heavy or light chain, thereby enabling the reconstitution of a bispecific, single-chain antibody, two-chain antibody (or single chain or two-chain fragment thereof such as Fab) having a binding specificity a first PDGF/VEGF molecule and a second PDGF/VEGF molecule. Construction and cloning of such a gene construct can be performed by standard procedures.

Bispecific antibodies are also generated via phage display screening methods using the so-called hierarchical dual combinatorial approach as disclosed in WO 92/01047 in which an individual colony containing either an H or L chain clone is used to infect a complete library of clones encoding the other chain (L or H) and the resulting two-chain specific binding member is selected in accordance with phage display techniques such as those described therein. This technique is also disclosed in Marks et al, (Bio/Technology, 10:779-783, 1992).

Recombinant antibody fragments, e.g. scFvs, can also be engineered to assemble into stable multimeric oligomers of high binding avidity and specificity to different target antigens. Procedures for making such diabodies (dimers), triabodies (trimers) or tetrabodies (tetramers) are well known within the art and have been described in the literature, see e.g., Kortt et al., (Biomol Eng. 18:95-108, 2001) and Todorovska et al., (J Immunol Meth. 248:47-66, 2001), and can be performed in mammalian cells using recombinant methods. See, e.g., Mack et al., Proc. Natl. Acad. Sci., 92:7021-7025, 1995, incorporated herein by reference.

Antigen-specific single chain antibody fragments are also identified by screening antibody phage display libraries, which typically comprise either immunoglobulin variable heavy chain fragments or immunoglobulin variable light chain fragments. The phage library is transfected into host cells, and phage particles expressing antibody fragments are isolated using techniques common in the art (e.g., Fredericks et al., Protein Engineering, Design and Selection 17:95-106, 2004; Zavala et al., Nuc. Acids Res. 28:E24, 2000). The library of isolated phage particles is then screened by panning, wherein the phage particles is cultured with antigen to detect antigen-specific binding (see e.g., Zavala, supra; Chowdury et al, Proc. Natl. Acad. Sci., USA. 95:669-74, 1998). For example, panning may be performed using antigen coated tubes, ELISA, antigen coated beads, or biotinylated antigen and streptavidin coated beads. Phage particles that express an antibody with affinity for the antigen are isolated by isolating the antigen coated substrate (e.g., bead) to which they bound. Continued rounds of panning enables isolation of antibodies with increased binding affinities.

Selected clones producing either V_{H} or V_{L} high affinity scFv are then cloned and pooled. The V_{H} or V_{L} pooled libraries undergo chain shuffling using techniques common in the art to generate clones expressing Fab fragments comprising a heavy and light chain specific for the antigen. See for example, Zhou et al., Proc. Natl. Acad. Sci., USA. 99:5241-5246, 2002.

In one aspect, bispecific antibodies (bscAb) are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNA's obtained from each hybridoma are then joined to form a single-chain fragment in a two-step fusion PCR. Bispecific fusion proteins are prepared in a similar manner. Bispecific single-chain antibodies and bispecific fusion proteins are antibody substances included within the scope of the present invention.

Further recent methods for producing bispecific monoclonal antibodies (mAbs) include engineered recombinant mAbs which have additional cysteine residues so that they crosslink more strongly than the more common immunoglobulin isotypes. See, e.g., FitzGerald et al, Protein Eng. 10:1221-1225, 1997. Another approach is to engineer recombinant fusion proteins linking two or more different single-chain antibody or antibody fragment segments with the desired dual specificities. See, e.g., Coloma et al., Nature Biotech. 15:159-163, 1997. A variety of bispecific fusion proteins can be produced using molecular engineering. In one form, the bispecific fusion protein is monovalent, consisting of, for example, a scFv having a binding site for one antigen and a Fab fragment having a binding site for a second antigen. In another form, the bispecific fusion protein is divalent, consisting of, for example, an IgG with two binding sites for one antigen, and two scFv with two binding sites for a second antigen.

Recombinant methods can be used to produce a variety of fusion proteins. For example a fusion protein comprising a Fab fragment derived from a first monoclonal antibody and a scFv derived from a second monoclonal antibody can be produced. A flexible linker, such as GGGS connects the scFv of the second antibody to the constant region of the heavy chain of the first antibody Fab. Alternatively, the scFv of the second antibody can be connected to the constant region of the light chain of the first antibody. Appropriate linker sequences necessary for the in-frame connection of the heavy chain Fd (antibody fragment comprising the heavy chain variable and CH3 constant region) to the scFv are introduced into the V_{L} (variable lambda light chain) and/or V_{K} (variable kappa light chain) domains through PCR reactions. The DNA fragment encoding the second antibody scFv is then ligated into a staging vector containing a DNA sequence encoding a CH1 domain. The resulting scFv-CH1 construct is excised and ligated into a vector containing a DNA sequence encoding the V_{H} region of the first monoclonal antibody. The resulting vector can be used to transfect an appropriate host cell, such as a mammalian cell for the expression of the bispecific fusion protein.

An example of use of transcription/translation to produce a single polypeptide chain bispecific antibody is as follows. Certain animals (camels; llamas; dromedaries) produce heavy chain antibodies, where there is no associated light chain. These antibodies have a single variable region, which can bind to antigen. Recombinant bispecific antibodies comprising two variable regions (from two different heavy chain antibodies) plus a linker region (L_{H}, from llama upper hinge) have been produced. The resulting complex (V_{H1}-L_{H}-V_{H2}) can be expressed in bacteria (Conrath et al., J. Biol. Chem. 276:7346-50, 2001). Humanized counterparts of the bispecific antibodies based on camel heavy chain antibodies are contemplated.

Alternatively, the bispecific antibodies may be V_{L} or V_{H} antibody domains which are maintained as peptide fragments known as domain antibodies (dAb), and are similar to camel-derived antibodies (Holt et al., Trends Biotechnol. 21:484-90, 2003). Each V_{L} or V_{H} comprises three antigen specific CDR. A domain antibody may be selected using phage display or other techniques well known in the art (Holt et al., supra). The domain antibody may be modified as necessary, such as by extending the length of the CDR loops or modifying the amino acid sequence, to improve stability and expression of the dAb (Holt et al, supra).

Single chain variable fragments (scFᵥ) have been connected to each other to form a bispecific antibody by various techniques: cross-linking C-terminal cysteine residues, adding naturally associating helices from a four-helix bundle, adding leucine zippers, adding a CH3 domain with either a knob or hole at the interacting surfaces, or by connecting CH1 and CL domains to the respective scFV fragments (Conrath, et al., J. Biol. Chem. 276:7346, 2001).

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701, 1984; Titus et al., J. Immunol., 138:4018-22, 1987).

Oligopeptides and polypeptides may be used for linking two different antibodies or antibody chains together. Oligo- and polypeptides may be synthesized by solution phase or by solid phase techniques. These include processes such as are described in Stewart and Young, Solid Phase Peptide Synthesis, Pierce Chemical Co., Rockford, Ill. (1984); Bodanszky, The Principles of Peptide Synthesis, 2nd ed., Springer, New York (1993); and Molina et al., Pept. Res. 9:151-5, 1996. For example, an azide process, an acid chloride process, an acid anhydride process, a mixed anhydride process, an active ester process (for example, p-nitrophenyl ester, N-hydroxy-succinimide ester, or cyanomethyl ester), a carbodiimidazole process, an oxidative-reductive process, or a dicyclohexylcarbodiimide (DCCD)/additive process can be used.

Also included are bispecific linear molecules, such as the so-called “Janusin” structures described by Traunecker et al. (EMBO J. 10:3655-9, 1991). This can be accomplished by genetically removing the stop codons at the end of a gene encoding a monomeric single-chain antigen-binding protein and inserting a linker and a gene encoding a second single-chain antigen-binding protein (WO 93/11161).

In a further approach, bispecific antibodies are formed by linking component antibodies to leucine zipper peptides (Kostelny et al., J. Immunol. 148:1547-53, 1992; de Kruif and Logtenberg, J. Biol. Chem. 271, 7630-4, 1996). Leucine zippers have the general structural formula (Leucine-X₁-X₂-X₃-X₄-X₅-X₆)ₙ, where X may be any of the conventional 20 amino acids (Creighton. Proteins, Structures and Molecular Principles, W. H. Freeman and Company, New York (1984)), but are most likely to be amino acids with high alpha-helix forming potential, for example, alanine, valine, aspartic acid, glutamic acid, and lysine (Richardson and Richardson, Science 240:1648-52, 1988, Erratum in: Science 242:1624, 1988), and n may be 3 or greater, although typically n is 4 or 5. The leucine zipper occurs in a variety of eukaryotic DNA-binding proteins, such as GCN4, C/EBP, c-fos gene product (Fos), c-jun gene product (Jun), and c-myc gene product. In these proteins, the leucine zipper creates a dimerization interface wherein proteins containing leucine zippers may form stable homodimers and/or heterodimers.

The leucine zippers for use in the present invention preferably have pairwise affinity. Pairwise affinity is defined as the capacity for one species of leucine zipper, for example, the Fos leucine zipper, to predominantly form heterodimers with another species of leucine zipper, for example, the Jun leucine zipper, such that heterodimer formation is preferred over homodimer formation when two species of leucine zipper are present in sufficient concentrations (Schuemann, et al., Nucleic Acids Res. 19:739-46, 1991). Thus, predominant formation of heterodimers leads to a dimer population that is typically 50 to 75 percent, preferentially 75 to 85 percent, and most preferably more than 85 percent heterodimers. When amino-termini of the synthetic peptides each include a cysteine residue to permit intermolecular disulfide bonding, heterodimer formation occurs to the substantial exclusion of homodimerization.

In a further embodiment, the bispecific antibody of the invention may be formulated as an “anticalin”, a recombinant form of lipocalin modified to bind to a molecule of interest. Lipocalins constitute a family of proteins for storage or transport of hydrophobic and/or chemically sensitive organic compounds, for example the retinol-binding protein. It has been demonstrated that the bilin-binding protein, a member of the lipocalin family originating from the butterfly Pieris brassicae, can be structurally reshaped in order to specifically complex potential antigens. Lipocalin share a conserved β-barrel, which is made of eight antiparallel β-strands, winding around a central core. At the wider end of the conical structure, these strands are connected in a pairwise manner by four loops that form the ligand binding site. The lipocalin scaffold can be employed for the construction of anticalins, which are made by individualizing various amino acid residues, distributed across the four loops, to targeted random mutagenesis. The production of anticalins is described further in International Patent Publ. WO99/16873 and in Beste et al., Proc. Natl. Acad, Sci. USA, 96:1898-1903, 1999.

In another aspect, the bispecific antibody need not be derived from a monoclonal antibody specific for a growth factor, but may be designed to bind to a common sequence in the two growth factors being bound. For instance, while all PDGF/VEGF family members by definition possess a region of high homology in the VEGF homology domain, certain growth factors may exhibit a greater degree of homology over particular regions of amino acids that would allow those growth factors to be bound specifically at this common region. For example, PDGF-C and PDGF-D possess a CUB domain with high homology that may be a target for a bispecific antibody that binds the CUB domain. Alternatively, these molecules exhibit a unique three amino acid insert in the VHD between conserved cysteines 3 and 4. This is NCA in PDGF-C and NCG in PDGF-D. A bispecific antibody could be designed with a binding site specific for the PDGF-C/PDGF-D VHD domain at this unique site. Unique homologous regions in a pair of PDGF/VEGF molecules may be determined by software programs and protein mapping techniques common in the art.

The invention also contemplates the use of multivalent antibody substances that are specific for three or more growth factors. Multivalent antibody substances are comprised of multiple single chain variable fragments (scF_{V}) each of which specifically binds a different antigenic target. For example, a multivalent antibody may be specific for all growth factors that bind to either VEGFR-1, VEGFR-2, VEGFR-3 or PDGFR. These scFv are assembled using chemical and/or peptide linkers into trivalent, tetravalent and larger multivalent antibodies using techniques common in the art, [see e.g. Lo, Benny K. C. (Ed.), Antibody Engineering Methods and Protocols, (Humana Press, Totowa, N.J., 2003)]. The multivalent antibody substances may be assembled tandemly, linearly, or as larger globular fusion proteins, which may include an Fc region or other antibody portion.

Antibody and Bispecific Antibody Substance Variants

Once an antibody or bispecific antibody substance had been prepared, its binding properties, stability, or other properties can optionally be improved by altering its amino acid sequence and screening for improved properties. Amino acid sequence variants of the polypeptide can be substitutional, insertional or deletion variants. Deletion variants lack one or more residues of the native protein which are not essential for function or immunogenic activity. Insertional mutants typically involve the addition of material at a non-terminal point in the polypeptide.

Variants may be substantially homologous or substantially identical to the bispecific antibody described below. Preferred variants are those which are variants of a bispecific antibody polypeptide which retain at least some of the biological activity, e.g., VEGF-D binding activity, of the bispecific antibody.

Substitutional variants typically exchange one amino acid of the wild-type for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge, as described above.

Polynucleotide variants and antibody fragments may be readily generated by a worker of skill to encode biologically active fragments, variants, or mutants of the naturally occurring antibody molecule that possess the same or similar biological activity to the naturally occurring antibody. This may be done by PCR techniques, cutting and digestion of DNA encoding the antibody heavy and light chain regions, and the like. For example, point mutagenesis, using PCR and other techniques well-known in the art, may be employed to identify with particularity which amino acid residues are important in particular activities associated with antibody activity. Thus, one of skill in the art will be able to generate single base changes in the DNA strand to result in an altered codon and a missense mutation.

Two manners for defining genera of polypeptide variants include minimum percent amino acid identity to the amino acid sequence of a preferred polypeptide (e.g., at least 80, 85, 90, 91, 92, 93, 94, 95, 96, 97, 98, or 99% identity preferred), or the ability of encoding-polynucleotides to hybridize to each other under specified conditions. One exemplary set of conditions is as follows: hybridization at 42° C. in 50% formamide, 5×SSC, 20 mM Na.PO4, pH 6.8; and washing in 1×SSC at 55° C. for 30 minutes. Formula for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

One aspect of the present invention contemplates generating glycosylation site mutants in which the O- or N-linked glycosylation site of the bispecific antibody has been mutated. Such mutants will yield important information pertaining to the biological activity, physical structure and substrate binding potential of the bispecifc antibody. In particular aspects it is contemplated that other mutants of the bispecific antibody polypeptide may be generated that retain the biological activity but have increased or decreased substrate binding activity. As such, mutations of the antigen-binding site are particularly contemplated in order to generate protein variants with altered binding activity.

In order to construct mutants such as those described above, one of skill in the art may employ well known standard technologies. Specifically contemplated are N-terminal deletions, C-terminal deletions, internal deletions, as well as random and point mutagenesis.

N-terminal and C-terminal deletions are forms of deletion mutagenesis that take advantage for example, of the presence of a suitable single restriction site near the end of the C- or N-terminal region. The DNA is cleaved at the site and the cut ends are degraded by nucleases such as BAL31, exonuclease III, DNase I, and S1 nuclease. Rejoining the two ends produces a series of DNAs with deletions of varying size around the restriction site. Proteins expressed from such mutant can be assayed for appropriate biological function, e.g., enzymatic activity, using techniques standard in the art, and described in the specification. Similar techniques may be employed for internal deletion mutants by using two suitably placed restriction sites, thereby allowing a precisely defined deletion to be made, and the ends to be religated as above.

Also contemplated are partial digestion mutants. In such instances, one of skill in the art would employ a “frequent cutter”, that cuts the DNA in numerous places depending on the length of reaction time. Thus, by varying the reaction conditions it will be possible to generate a series of mutants of varying size, which may then be screened for activity:

A random insertional mutation may also be performed by cutting the DNA sequence with a DNase I, for example, and inserting a stretch of nucleotides that encode, 3, 6, 9, 12 etc., amino acids and religating the end. Once such a mutation is made the mutants can be screened for various activities presented by the wild-type protein.

The amino acids of a particular protein can be altered to create an equivalent, or even an improved, second-generation molecule. Such alterations contemplate substitution of a given amino acid of the protein without appreciable loss of interactive binding capacity with structures such as, for example, antigen-binding regions of antibodies or binding sites on substrate molecules or receptors. Since it is the interactive capacity and nature of a protein that defines that protein's biological functional activity, certain amino acid substitutions can be made in a protein sequence, and its underlying DNA coding sequence, and nevertheless obtain a protein with like properties. Thus, various changes can be made in the DNA sequences of genes without appreciable loss of their biological utility or activity, as discussed below.

In making such changes, the hydropathic index of amino acids may be considered. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte & Doolittle, J. Mol. Biol., 157:105-132, 1982, incorporated herein by reference). Generally, amino acids may be substituted by other amino acids that have a similar hydropathic index or score and still result in a protein with similar biological activity, i.e., still obtain a biological functionally equivalent protein.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

Exemplary amino acid substitutions that may be used in this context of the invention include but are not limited to exchanging arginine and lysine; glutamate and aspartate; serine and threonine; glutamine and asparagine; and valine, leucine and isoleucine. Other such substitutions that take into account the need for retention of some or all of the biological activity whilst altering the secondary structure of the protein will be well known to those of skill in the art.

Treatment of Cancer Using the Methods and Compositions of the Invention

The present invention provides methods of treating cancer in an animal, comprising administering to the animal an effective amount of a composition comprising an antibody substance of the invention. The invention is similarly directed to methods of inhibiting cancer cell growth, including processes of cellular proliferation, invasiveness, and metastasis in biological systems. The antibody substances inhibit or reduce cancer cell growth, invasiveness, metastasis, or tumor incidence in living animals, such as mammals.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of cells in which the multiplication of the cells is uncontrolled and progressive. This is also referred to as neoplastic cell growth. Some such growths are benign, but others are malignant and may lead to death of the organism. Malignant neoplasms or cancers are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention include solid tumors, for example, carcinomas and sarcomas. Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. The invention also provides methods of treatment of cancers of myeloid or lymphoid systems, including leukemias, lymphomas, and other cancers that typically are not present as a tumor mass, but are distributed in the vascular or lymphoreticular systems.

Further contemplated are methods for treatment of adult and pediatric oncology, growth of solid tumors/malignancies, myxoid and round cell carcinoma, locally advanced tumors, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma of the lungs, cutaneous T cell lymphoma, Hodgkin's lymphoma, non-Hodgkin's lymphoma, cancer of the adrenal cortex, ACTH-producing tumors, non-small cell lung cancers, breast cancer, including small cell carcinoma and ductal carcinoma), gastro-intestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, basal cell carcinoma, and squamous cell cancer), thyroid cancer, retinoblastoma, neuroblastoma, peritoneal effusion, malignant pleural effusion, mesothelioma, Wilms's tumors, gall bladder cancer, trophoblastic neo-plasms, hemangiopericytoma, and Kaposi's sarcoma.

Any chemotherapeutic or radiotherapeutic agent may be suitable for use in combination with the antibody substance of the invention in a method of the invention, and may be identified by means well known in the art. Examples of suitable chemotherapeutic and radiotherapeutic agents include, but are not limited to: an anti-metabolite; a DNA-damaging agent; a cytokine or growth factor useful as a chemotherapeutic agent; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radiosensitizer; a photosensitizer; a radiation source, optionally together with a radiosensitizer or photosensitizer; or other commonly used therapeutic agents. Compositions and kits comprising an antibody substance of the invention with one or more of these agents is a further aspect of the invention.

Specific examples of chemotherapeutic agents useful in methods of the present invention are listed in Table 1.

TABLE 1

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenimine/Methylmelamine
thriethylenemelamine (TEM)
triethylene
thiophosphoramide (thiotepa)
hexamethylmelamine (HMM, altretamine)
Alkyl sulfonates
busulfan
Triazines
dacarbazine (DTIC)
Antimetabolites
Folic Acid analogs
methotrexate
Trimetrexate
Pemetrexed
Multi-targeted antifolate
Pyrimidine analogs
5-fluorouracil
fluorodeoxyuridine
gemcitabine
cytosine arabinoside (AraC, cytarabine)
5-azacytidine
2,2′-difluorodeoxy-cytidine
Purine analogs
6-mercaptopurine
6-thioguanine
azathioprine
2′-deoxycoformycin (pentostatin)
erythrohydroxynonyl-adenine (EHNA)
fludarabine phosphate
2-chlorodeoxyadenosine (cladribine, 2-CdA)
Type I Topoisomerase Inhibitors
camptothecin
topotecan
irinotecan
Natural products
Antimitotic drugs
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycinsplicamycin (mithramycin)
mitomycinC
dactinomycin
Enzymes
L-asparaginase
Biological response modifiers
interferon-alpha
IL-2
G-CSF
GM-CSF
Differentiation Agents
retinoic acid
derivatives
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinium coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o,p′-DDD)
ainoglutethimide
Cytokines
interferon (*, *, *)
interleukin-2
Hormones and antagonists
Adrenocorticosteroids/antagonists
prednisone and equivalents
dexamethasone
ainoglutethimide
Progestins
hydroxyprogesterone
caproate
medroxyprogesterone
acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing hormone analogs
leuprolide
Nonsteroidal antiandrogens
flutamide
Photosensitizers
hematoporphyrin
derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines

Bispecific antibody compositions administered may also include cytokines and growth factors that are effective in inhibiting tumor metastasis, and wherein the cytokine or growth factor has been shown to have an antiproliferative effect on at least one cell population. Such cytokines, lymphokines, growth factors, or other hematopoietic factors include M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, erythropoietin. Additional growth factors for use in pharmaceutical compositions of the invention include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

Advantageously, when a second agent is used in combination with the bispecific antibodies of the present invention, the results obtained are synergistic. That is to say, the effectiveness of the combination therapy of a bispecific antibody and the second agent is synergistic, i.e., the effectiveness is greater than the effectiveness expected from the additive individual effects of each. Therefore, the dosage of the second agent can be reduced and thus, the risk of the toxicity problems and other side effects is concomitantly reduced.

Other PDGF/VEGF Growth Factor Mediated Diseases

Studies have demonstrated that signaling through the PDGF/VEGF family of receptors, or interference with signaling through the PDGFRs and VEGFRs, has significant effect on vascular development, angiogenesis, and lymphangiogenesis. Interference with the ligands of these receptors is one approach to inhibiting these biological processes. In practice however, it may be difficult to inhibit a receptor in vivo using this approach when multiple growth factors are capable of stimulating the receptor. The present invention addresses that need and provides agents that can block multiple growth factors that signal through a target receptor.

For example, VEGF-B and PlGF both bind to VEGFR-1. Blocking the ability of both VEGF-B and PlGF to bind the VEGFR-1 receptor blocks downstream reactions of VEGFR-1 which lead to angiogenesis. VEGF-B has been implicated in the progression of rheumatoid arthritis (Mould et al., Arthritis Rheum. 48:2660-9, 2003), wherein Vegfb knockout mice experience reduced pathology and synovial angiogenesis. Additionally, PlGF has been thought to play a role in angiogenesis in inflammatory mediated diseases (Autiero et al., J Thromb Haemost. 1: 1356-70, 2003). PlGF has also been implicated in edema, vascular leakage, tumor formation, pulmonary hypertension, inflammatory disorders, and ischemic retinopathy. Thus, a VEGF-B/PlGF bispecific antibody which blocks activity through the VEGFR-1 receptor has therapeutic indications in a wide range of diseases.

VEGF-A and VEGF-B form natural heterodimers in vivo and are implicated as a major factor in the progression of angiogenesis. Antibodies specific for VEGF-A and VEGF-B are contemplated. The bispecific antibody preferably also binds VEGF-A isoforms dimerized VEGF-B isoforms. It is also contemplated that antibodies that bind either VEGF-A/VEGF-B₁₆₇ and not VEGF-A/VEGF-B₁₈₆, and vice versa, may be generated, and used therapeutically in diseases wherein inhibition of one VEGF-B isoform is preferable.

VEGF-A-overexpressing transgenic mice showed an increased vascularization with edema due to hyper-vascular permeability and subcutaneous hemorrhage as side effects (Kiba et al., Biochem Biophys Res Commun. 301:371-7, 2003). Similarly, VEGF-E overexpression in mice leads to hypervascularization, with reduced edema compared to VEGF-A overexpresison. VEGF-E has been implicated in cardiovascular or endothelial disorders, such as cardiac hypertrophy, arterial disease, such as atherosclerosis, hypertension, inflammatory vasculitis and myocardial infarction. Both VEGF-A and VEGF-E bind with high affinity to the VEGFR-2 molecule, and VEGFR-2 has been shown to mediate most of the endothelial growth and survival signals from VEGF-A. These results suggest that to completely block signaling through VEGFR-2, in some circumstances, such as infections or disorders characterized by VEGF-E expression, the activity of both VEGF-A and VEGF-E must be inhibited. The present invention contemplates use of a bispecific VEGF-A/VEGF-E antibody that blocks VEGFR-2 signaling and thereby reduces vascularization of tumor cells and edema related to hypervascularization.

Both PDGF-C and PDGF-D have been implicated as potent angiogenic factors (Li et al., Oncogene 22:1501-10, 2003), and have been observed to be upregulated in brain tumors such as glioblastomas (Lokker et al., Cancer Res. 62:3729-35, 2002), which also express PDGFRs. Thus, administration of PDGF-C/PDGF-D bispecific antibodies to patients with different cancers, including but not limited to glioblastoma, will reduce the angiogenic effects of signaling through the PDGFR.

Recent evidence on the association of lymphangiogenic growth factors with intralymphatic growth and metastasis of cancers (PCT/US99/23525; WO 02/060950; Mandriota, et al., EMBO J. 20:672-682, 2001; Skobe et al., Nat. Med. 7:192-198, 2001; Stacker et al., Nat. Med. 7:186-191, 2001; Karpanen et al., Cancer Res. 61:1786-1790, 2001) has provided an indication for anti-lymphangiogenic agents for tumor therapy. VEGF-C and VEGF-D signaling through the VEGFR-3 receptor has been shown to be the primary source of lymphangiogenic activation and has also been noted in pathogenic angiogenesis in some tumors.

Cancer cells spread within the body by direct invasion to surrounding tissues, spreading to body cavities, invasion into the blood vascular system (hematogenous metastasis), as well as spread via the lymphatic system (lymphatic metastasis). Regional lymph node dissemination is the first step in the metastasis of several common cancers and correlates highly with the prognosis of the disease. The lymph nodes that are involved in draining tissue fluid from the tumor area are called sentinel nodes, and diagnostic measures are in place to find these nodes and to remove them in cases of suspected metastasis. Blockade of signals through VEGFR-3 using neutralizing antibodies bispecific for VEGF-C and VEGF-D will reduce the extent of angiogenesis and lymphangiogenesis, and consequently reduce tumor metastasis through the lymph system.

Derivatives

As stated above, derivative refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine. Derivatives of the antibody substance of the invention, such as a bispecific antibody, are also useful as therapeutic agents and may be produced by the method of the invention

The detectable moiety can be incorporated in or attached to an antibody substance either covalently, or through ionic, van der Waals or hydrogen bonds, e.g., incorporation of radioactive nucleotides, or biotinylated nucleotides that are recognized by streptavadin.

Polyethylene glycol (PEG) may be attached to the antibody substances to provide a longer half-life in vivo. The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5 kDa to about 10 kDa. The PEG groups will generally be attached to the antibody substances of the invention via acylation or reductive alkylation through a natural or engineered reactive group on the PEG moiety (e.g., an aldehyde, amino, thiol, or ester group) to a reactive group on the antibody substance (e.g., an aldehyde, amino, or ester group). Addition of PEG moieties to antibody substances can be carried out using techniques well-known in the art. See, e.g., International Publication No. WO 96/11953 and U.S. Pat. No. 4,179,337.

Ligation of the antibody substance with PEG usually takes place in aqueous phase and can be easily monitored by reverse phase analytical HPLC. The PEGylated substances are purified by preparative HPLC and characterized by analytical HPLC, amino acid analysis and laser desorption mass spectrometry.

Labels

In some embodiments, the antibody substance is labeled to facilitate its detection. A “label” or a “detectable moiety” is a composition detectable by spectroscopic, photochemical, biochemical, immunochemical, chemical, or other physical means. For example, labels suitable for use in the present invention include, radioactive labels (e.g., ³²P), fluorophores (e.g., fluorescein), electron-dense reagents, enzymes (e.g., as commonly used in an ELISA), biotin, digoxigenin, or haptens as well as proteins which can be made detectable, e.g., by incorporating a radiolabel into the hapten or peptide, or used to detect antibodies specifically reactive with the hapten or peptide.

Examples of labels suitable for use in the present invention include, but are not limited to, fluorescent dyes (e.g., fluorescein isothiocyanate, Texas red, rhodamine, and the like), radiolabels (e.g., ³H, ¹²⁵, ³⁵S, ¹⁴C, or ³²P), enzymes (e.g., horse radish peroxidase, alkaline phosphatase and others commonly used in an ELISA), and colorimetric labels such as colloidal gold, colored glass or plastic beads (e.g., polystyrene, polypropylene, latex, etc.).

The label may be coupled directly or indirectly to the desired component of the assay according to methods well known in the art. Preferably, the label in one embodiment is covalently bound to the biopolymer using an isocyanate reagent for conjugation of an active agent according to the invention. In one aspect of the invention, the bifunctional isocyanate reagents of the invention can be used to conjugate a label to a biopolymer to form a label biopolymer conjugate without an active agent attached thereto. The label biopolymer conjugate may be used as an intermediate for the synthesis of a labeled conjugate according to the invention or may be used to detect the biopolymer conjugate. As indicated above, a wide variety of labels can be used, with the choice of label depending on sensitivity required, ease of conjugation with the desired component of the assay, stability requirements, available instrumentation, and disposal provisions. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The compounds of the invention can also be conjugated directly to signal-generating compounds, e.g., by conjugation with an enzyme or fluorophore. Enzymes suitable for use as labels include, but are not limited to, hydrolases, particularly phosphatases, esterases and glycosidases, or oxidotases, particularly peroxidases. Fluorescent compounds, i.e., fluorophores, suitable for use as labels include, but are not limited to, fluorescein and its derivatives, rhodamine and its derivatives, dansyl, umbelliferone, etc. Further examples of suitable fluorophores include, but are not limited to, eosin, TRITC-amine, quinine, fluorescein W, acridine yellow, lissamine rhodamine, B sulfonyl chloride erythroscein, ruthenium (tris, bipyridinium), Texas Red, nicotinamide adenine dinucleotide, flavin adenine dinucleotide, etc. Chemiluminescent compounds suitable for use as labels include, but are not limited to, luciferin and 2,3-dihydrophthalazinediones, e.g., luminol. For a review of various labeling or signal producing systems that can be used in the methods of the present invention, see U.S. Pat. No. 4,391,904.

Means for detecting labels are well known to those of skill in the art. Thus, for example, where the label is radioactive, means for detection include a scintillation counter or photographic film, as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Other labeling and detection systems suitable for use in the methods of the present invention will be readily apparent to those of skill in the art. Such labeled modulators and ligands can be used in the diagnosis of a disease or health condition.

Drug Delivery

The invention contemplates attachment of a drug payload to antibodies of the invention. Prodrug design is discussed generally in Hardma et al. (Eds.), Goodman and Gilman's The Pharmacological Basis of Therapeutics, 9th ed., pp. 11-16 (1996). Prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Formulation of Pharmaceutical Compositions

To administer antibody substances of the invention to human or test animals, it is preferable to formulate the antibody substances in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

The antibody substance and bispecific antibody substance compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient. Injection, especially intravenous and intratumoral, are preferred.

Pharmaceutical compositions of the present invention containing an antibody substance of the invention as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

Formulation of the pharmaceutical composition will vary according to the route of administration selected (e.g., solution, emulsion). An appropriate composition comprising the humanized antibody to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The antibodies of this invention can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

The concentration of antibody in these formulations can vary widely, for example from less than about 0.5%, usually at or at least about 1% to as much as 15 or 20% by weight and will be selected primarily based on fluid volumes, viscosities, etc., in accordance with the particular mode of administration selected. Thus, a typical pharmaceutical composition for parenteral injection could be made up to contain 1 ml sterile buffered water, and 50 mg of antibody. A typical composition for intravenous infusion could be made up to contain 250 ml of sterile Ringer's solution, and 150 mg of antibody. Actual methods for preparing parenterally administrable compositions will be known or apparent to those skilled in the art and are described in more detail in, for example, Remington's Pharmaceutical Science, 15th ed., Mack Publishing Company, Easton, Pa. (1980). An effective dosage of bispecific antibody is within the range of 0.01 mg to 1000 mg per kg of body weight per administration.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

Bispecific antibody compositions contemplated for use inhibit cancer growth, including proliferation, invasiveness, and metastasis, thereby rendering them particularly desirable for the treatment of cancer. In particular, the compositions exhibit cancer-inhibitory properties at concentrations that are substantially free of side effects, and are therefore useful for extended treatment protocols. For example, co-administration of a bispecific antibody composition with another, more toxic, chemotherapeutic agent can achieve beneficial inhibition of a cancer, while effectively reducing the toxic side effects in the patient.

In addition, the properties of hydrophilicity and hydrophobicity of the compositions contemplated for use in the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action. Thus, bispecific antibody compositions contemplated are maximally effective when they can be delivered to the site of the tumor and they enter the tumor cells.

Administration and Dosing

In one aspect, methods of the invention include a step of administration of a pharmaceutical composition.

Methods of the invention are performed using any medically-accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the cancer.

Therapeutic compositions may also be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly or monthly.

Particularly contemplated in the presenting invention is the administration of multiple agents, such as a bispecific antibody in conjunction with a second agent as described herein. It is contemplated that these agents may be given simultaneously, in the same formulation. It is further contemplated that the agents are administered in a separate formulation and administered concurrently, with concurrently referring to agents given within 30 minutes of each other.

In another aspect, the second agent is administered prior to administration of the bispecific antibody. Prior administration refers to administration of the second agent within the range of one week prior to treatment with the bispecific antibody, up to 30 minutes before administration of the bispecific antibody. It is further contemplated that the second agent is administered subsequent to administration of the bispecific antibody. Subsequent administration is meant to describe administration from 30 minutes after bispecific antibody treatment up to one week after bispecific antibody administration.

It is further contemplated that when bispecific antibody is administered in combination with a second agent, wherein the second agent is a cytokine or growth factor, or a chemotherapeutic agent, the administration also includes use of a radiotherapeutic agent or radiation therapy. The radiation therapy administered in combination with a bispecific antibody composition is administered as determined by the treating physician, and at doses typically given to patients being treated for cancer.

The amounts of bispecific antibody in a given dosage will vary according to the size of the individual to whom the therapy is being administered as well as the characteristics of the disorder being treated. In exemplary treatments, it may be necessary to administer about 1 mg/day, 5 mg/day, 10 mg/day, 20 mg/day, 50 mg/day, 75 mg/day, 100 mg/day, 150 mg/day, 200 mg/day, 250 mg/day, 500 mg/day or 1000 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with therapeutics of the invention.

Kits

As an additional aspect, the invention includes kits which comprise one or more compounds or compositions packaged in a manner which facilitates their use to practice methods of the invention. In one embodiment, such a kit includes a compound or composition described herein (e.g., a composition comprising a bispecific antibody alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the bispecific antibody composition.

Additional aspects and details of the invention will be apparent from the following examples. Example 1 describes generation of hybrid hybridomas to make antibody substances specific for multiple PDGF/VEGF molecules. Example 2 describes generating a cross-reacting bispecific antibody for two growth factors. Example 3 describes an assay to measure antibody substance binding specificity. Example 4 describes identification of VEGF-B antibodies that cross-react with VEGF-A. Example 5 describes an assay to measure antibody substance neutralization of target growth factor activity. Example 6 describes assays useful for measuring PDGF/VEGF bispecific antibody inhibition/blockade of receptor signaling. Example 7 describes in vitro and in vivo angiogenesis assays to assess bispecific antibody binding. Example 8 describes assays to measure the effects of bispecific antibodies on growth factor mediated tumor growth and metastasis. Example 9 describes inhibition of VEGF-C binding to VEGFR-2 or VEGFR-3 by bispecific VEGF-C/VEGF-D antibody. Example 10 describes biological effects of PDGF-C/PDGF-D bispecific antibody. Example 11 describes construction of antibodies to regions of similarity in PDGF-C and PDGF-D. Example 12 describes screening a phage display library to detect bispecific antibodies. Example 13 discloses animal models to demonstrate the efficacy bispecific antibody therapies for treatment of cancers. Example 14 describes administration of bispecific antibody compositions to cancer patients.

### EXAMPLE 1

### Hybrid Hybridomas Specific for Multiple PDGF/VEGF Molecules

This protocol is used to generate bispecific antibodies specific for any combination of PDGF/VEGF molecules. Monoclonal antibodies specific for the targeted growth factors are made according to methods known in the art or described above. Two types of monoclonal antibodies, for example, specific for VEGF-B and PlGF; VEGF-A and VEGF-E; VEGF-A and VEGF-B; VEGF-C and VEGF-D; or PDGF-C and PDGF-D, are fused to form hybrid hybridomas as described below.

Hybrid hybridomas are formed by fusing two hybridomas according to the general polyethylene glycol (PEG) fusion protocol of Preffer et al., J. Immunol. 133:1857-62, 1984. See also, Reading EP 068763; Stearz and Bevan, Proc. Nat'l. Acad. Sci. USA 83:1453-1457, 1986; and Milstein et al., Nature 305:537-540, 1983. More specifically, one fusion partner is a hypoxanthine-guanine phosphoribosyltransferase (HGPRT)-deficient clone of one parental hybridoma (e.g., a VEGF-D-binding monoclonal antibody producer, such as 4A5, described in U.S. Pat. No. 6,383,484). HGPRT-deficient clones are obtained by growing the clones in increasing concentrations of 8-azaguanine (GIBCO, Grand Island, N.Y.; 1 μg/ml to 20 μg/ml).

The other fusion partner (for example a hybridoma that provides a VEGF-C-binding monoclonal antibody) is thymidine kinase (TK)-deficient, which is selectively grown in the presence of increasing concentrations of 5-bromodeoxyuridine (Sigma Chemical Co., St. Louis, Mo.; 3 μg/ml to 60 μg/ml).

The HGPRT deficient clone from one parental hybidoma is fused to TK deficient clone of the other parental hybridoma with PEG. After fusion, the desired hybrid hybridomas are selected in hypoxanthine-aminopterin-hymidine (HAT) supplemented media. The media consists of Isocove's modified Dulbecco's medium with 10% fetal calf serum, 2 mM glutamine, and 5 μg/ml gentamicin. Cells deficient in HGPRT and in TK complement each other and only the fused cells grow in the presence of HAT. Alternatively, the HAT-sensitive parenteral hybridomas may be additionally rendered neomycin-resistant by transfecting them with an incomplete retroviral vector containing the neomycin resistant gene. To produce the desired bispecific monoclonal antibody, one of these doubly selected parental hybridomas will be fused to the appropriate unselected (naturally HAT-resistant neomycin-sensitive) other parental hybridoma. Only the fused hybrid-hybridomas will survive in the selection medium containing G418 (GIBCO), a neomycin analogue, and HAT. A third method that may be used is based on a modification of the chemical hybridization method of Nisonoff and Palmer to obtain rapidly new bispecific monoclonal antibodies from the parental antibodies. 100 mM 2-mercaptoethanolamine is added to a mixture of the parental mAbs to reduce their inter-chain disulfide bonds. The reduced parental mAbs are then split into two half Ig molecules by disrupting the noncovalent inter-heavy chain bonds with buffers containing 25 mM NaCl at pH 2.5. The half Ig molecules are then allowed to reanneal randomly by dialyzing into a neutral PBS solution at pH 7.4.

Screening is accomplished by standard ELISA techniques with growth factor bound to a solid substrate or by indirect staining method consisting of incubating antigen expressing cells with the bispecific antibody at 4° C., followed by incubation with fluorescein isothiocyanate (FITC)-conjugated secondary antibodies. Antibody substance binding is also measurable by flow cytometry (Wong and Colvin, J. Immunol. 139:1369-1374, 1986). The screening is performed to confirm that the antibody generated by the method is specific for ands binds to both target antigens. Additional screening is performed to test the antibody substance's ability to neutralize the activity of the target growth factors. Activity screening-assays are described more fully below.

The bispecific monoclonal antibody is obtained either from the supernatant of the hybrid hybridoma or from the ascites fluid of mice injected with the hybrid hybridoma, and is purified by isoelectric focusing. Other purification techniques such as affinity chromatography using sequential mouse anti-idiotype anti-isotype monoclonal antibodies or high performance liquid chromatography may be used.

Monoclonal antibodies specific for the PDGF/VEGF molecules may be formulated according to other methods as described herein. The bispecific antibodies are then used in the following assays to determine their ability to interfere with PDGFR/VEGFR signaling and regulate angiogenesis.

### EXAMPLE 2

### Generating and Selecting a Cross-Reacting Bispecific Antibody to Two VEGF/PDGF Growth Factors

An antibody substance that specifically reacts with two (or more) members of the PDGF/VEGF growth factor family can be generated without recombination of antibodies by using appropriate selection techniques.

A general outline of the protocol is as follows:
(1) screening a library of antibody molecules to identify at least one antibody molecule that binds to a first VEGF/PDGF growth factor;
(2) screening molecule(s) identified by the first screening (1) to identify at least one molecule that binds to a second VEGF/PDGF growth factor;
(3) screening molecule(s) selected following the second screening step (2) for appropriate VEGF/PDGF neutralization activity, i.e., to identify at least one molecule that inhibits both the first and second growth factors to which it binds from stimulating phosphorylation of the receptors.
   1. Library of Antibody Molecules

Any library of antibody-like molecules can be employed. In one approach, a recombinant library, such as antibody substances in a phage display library, are employed.

In another approach, a laboratory animal is immunized with an antigen to generate antibodies (and antibody producing cells for making hybridomas). These antibodies, antibody producing cells, or hybridomas represent the library.

2. Antigen for Generating/Screening the Library

The antigen may comprise one of the growth factors of interest, or a peptide fragment thereof, or a peptide designed from knowledge of the sequences and/or knowledge or prediction of 3-D structures of the growth factors. For example, the amino acid sequences of the growth factors are aligned and regions are identified that are likely to be immunogenic (e.g., due to hydropathy analysis or molecular modeling) and that have stretches of amino acids that are conserved between the two proteins. In addition, three dimensional molecular modeling is used to identify regions that are exposed and that have similar structures and positions in two growth factors. The peptide antigen may be a synthetic peptide that comprises a sequence containing conserved residues at some positions, residues at other positions taken from the sequence of the first growth factor protein, and residues at other positions taken from the sequence of the second growth factor protein. Some residues may be substituted such that they are not identical to either protein, but are preferably conserved substitutions relative to a corresponding amino acid position in both growth factor sequences.

If the original antigen consists of one of the growth factors, then step (1) may be complete and the antibody substances that are generated against that antigen, or identified by screening with the antigen, are screened again against the other growth factor.

If, on the other hand, the original antigen is a peptide fragment of a growth factor, or a synthetic peptide designed from growth factor sequences, then the antibody substances that are generated against the antigen must be screened against both growth factors. (Steps 1 and 2.)

3. Neutralization Assay

Any of the activity assays described herein, including phosphorylation assays or cell growth/mobilization assays, may be used to screen a selected antibody substance for neutralization activity according to step 3.

4. Example: Generating a VEGF-A/VEGF-B Cross-Reacting Antibody

The amino acid sequences of human VEGF-A and human VEGF-B are aligned using conventional algorithms to identify regions of sequence similarity. A sequence of at least 6 consecutive, identical amino acids is preferred, with 7, 8, 9, 10, 11, 12, 13, 14, 15, or more being highly preferred. Mismatches within the selected peptide sequences preferably are still conservative substitution-type mismatches such that they are unlikely to interfere with cross-reactivity, e.g. where an acidic amino acid such as aspartic acid (D) in one protein aligns with glutamic acid (E) in the other; or where the side chains for the amino acids are unlikely to interfere with antibody cross-reactivity. Exemplary peptides include amino acids (aa) 87-110 of VEGF₁₆₅ (SEQ ID NO: 23), which correspond by way of alignment with amino acids(aa) 82-105 of VEGF-B₁₆₇ (SEQ ID NO: 24):

| | |
|---|---|
| CNDEGLECVPTEESNITMQIMRIK | (see VE GF-A, |
| | SEQ ID NO: 2, |
| | aa 87-110) |
| CPDDGLECVPTGQHQVRMQILMIR | (see VE GF-B, |
| | SEQ ID NO: 6, |
| | aa 82-105) |

or fragments of 5 or more amino acids from said peptides.

The selected peptides are used to immunize a laboratory animal using standard techniques to generate an immune response. In one preferred embodiment, a peptide from the VEGF-B sequence is used to immunize a VEGF-B knock-out (KO) mouse (described in WO 98/36052, incorporated herein by reference).

For example, a solution of recombinant human VEGF-B is emulsified in Complete Freunds Adjuvant and injected intraperitoneally (i.p.) into VEGF-B deficient mice (using 50-100 ug VEGF-B/mouse). Following repeated booster immunizations using the same amount of antigen, the spleen cells from animals who have developed a strong antigenic response (checked by determining the antibody titer in small blood samples obtained from the mice) are isolated and hybridomas are generated using standard technologies in the art. Aliquots of the growth media from the hybridomas are screened by ELISA techniques first using VEGF-B as the target antigen, and subsequently using VEGF-A as the target antigen. Hybridomas generating cross-reacting antibodies are further characterized to identify neutralizing antibodies using neutralization assays as described herein.

As an alternative method of generating monoclonal antibodies, the Immortomouse (containing one or two temperature-sensitive (ts) SV40 large T antigen allele(s) in their genome) are crossed with growth factor (e.g., VEGF-B) deficient mice, and the growth factor deficient mice containing at least one allele of the ts SV40 large T antigen (genotype VEGF-B−/−, ts SV40 largeT+) are used for immunization as above. To generate the antibody producing cells the technique outlined in Pasqualini and Arap is used (Pasqualini et al., Proc Natl Acad Sci USA. 101:257-259, 2004). Screening and other associated steps are carried out as above. It is contemplated that the cross-reactive antibodies identified by this or other techniques are humanized to decrease their antigenicity as a therapeutic.

An advantage of immunizing a VEGF-B KO mouse is that there should be a greater chance to get VEGF-B-specific antibodies: the antigen is truly foreign because the mouse has never had circulating murine VEGF-B in its system. In an alternative embodiment, a transgenic mouse that produces fully human immunoglobulins, such as Medarex's HuMab-Mouse™, is employed. In still another variation, a VEGF-B-knockout variant of such a transgenic mouse is employed.

While use of human peptides as antigen is preferred, use of peptides derived from growth factors of other species is possible, especially since there is known conservation in sequence between mammalian species for many of these growth factors.

Blood from animals that are producing cross-reactive antibodies is drawn and used to prepare hybridomas by standard techniques, and the hybridomas are again screened in the same manner to identify a hybridoma that produces a monoclonal antibody that cross-reacts with VEGF-A and VEGF-B. In preferred embodiments, the monoclonal antibodies are further screened to identify an antibody that binds both growth factors and inhibits both growth factors from causing phosphorylation of VEGFR-1 in cells that express VEGFR-1.

### EXAMPLE 3

### Assay for Binding Specificity

Assessing the ability of an antibody substance to bind its target antigens may be done using standard in vitro cell free assays such as an ELISA.

To test for antibody specificity for 2 antigens, for example if testing an antibody substance bispecific for VEGF-C and VEGF-D, a two-part ELISA is performed. First, Immulon 4 plates (Dynatech, Cambridge, Mass.) are coated for 2 hours at 37° C. with 100 ng/well of VEGF-C diluted in 25 mM Tris, pH 7.5. Standard ELISA washing and blocking techniques are performed before culture with 50 μl of solution comprising the antibody substance to be tested. The actual amount of antibody substance in the culture solution may vary depending on the antibody being tested. After incubation at 37° C. for 30 minutes, and washing as above, 50 μl of horseradish peroxidase conjugated goat anti-mouse IgG(Fc) (Jackson ImmunoResearch, West Grove, Pa.) diluted 1:3500 in PBST is added. Plates are incubated as above, washed four times with PBST, and 100 μl substrate, consisting of 1 mg/ml o-phenylene diamine (Sigma) and 0.1 μl/ml 30% H₂O₂ in 100 mM Citrate, pH 4.5, are added. Wells positive for the antigen will change color due to the enzymatic reaction. The color reaction is stopped after 5 minutes with the addition of 50 μl of 15% H₂SO₄. A490 is read on a plate reader (Dynatech). Antibody substances positive for the first antigen (e.g. VEGF-C) are then used in a second ELISA assay in which the plate wells are coated with the second target antigen (e.g. VEGF-D). Wells that are positive in the second reaction indicate an antibody substance that specifically binds both target antigens.

### EXAMPLE 4

### Identification of VEGF-B Antibodies that Cross-React with VEGF-A

Bispecific antibodies that bind to both VEGF-A and VEGF-B provide a useful therapeutic which can simultaneously neutralize VEGF-A and VEGF-B activity in various conditions relating to undesired angiogenesis, for example, in blocking tumor growth and inflammation.

The following procedures were performed to identify populations of antibodies in a polyclonal rabbit-antiserum generated against VEGF-B antigen that cross-react with VEGF-A.

Human recombinant VEGF-B₁₆₇ protein (75 ng per lane, Amrad) and human recombinant VEGF-A₁₆₅ (100 ng and 400 ng per lane, R&D System, 293-VE) were subjected to SDS-PAGE analysis under non-reducing, or reducing conditions in 12% SDS-PAGE gel. The gel was blotted on a PVDF membrane and the blot was blocked with 5% milk/PBS with 0.1% BSA at room temperature for 1 hour. The cross-activity of VEGF-B antibody with VEGF-A protein was detected using a 1:500 dilution of antiserum raised against mouse VEGF-B₁₈₆ at room temperature for 1 hour (Aase et al., Developmental Dynamics, 215, 12-25, 1999). The same blot was stripped and VEGF-A protein was detected using monoclonal anti-human VEGF-A antibody (R&D System, MAB 293) with a 2 μg/ml dilution. Bound antibodies were observed using ECL+ reagent (Amersham) and visualized using a CCD camera (Fuji).

Immunoblotting analysis showed that antibodies in the polyclonal rabbit antiserum reacted strongly with both reduced and non-reduced VEGF-B protein. The reactivity was less strong with VEGF-A, and was strongest against the reduced VEGF-A protein. As control, the same immunoblot was probed with a monoclonal antibody to VEGF-A. Strong reactivity was obtained with the non-reduced protein, whereas the epitope for this monoclonal antibody was lost under reducing conditions.

These results indicated that some populations of antibodies raised against VEGF-B will cross-react with VEGF-A. The ability of these antibodies to bind multiple VEGF family members provides a novel method for treating conditions associated with upregulation of both VEGF-B and VEGF-A activity.

Alternatively, the specific antibody within the antiserum that cross-reacts with VEGF-A and VEGF-B is isolated from the antiserum by affinity purification of Ig fractions using standard biochemical purification procedures. For example, the antiserum is passed over a column (Sepharose 4B or agarose) of immobilized recombinant VEGF-A (any isoform). The bound antibody is released by eluting it with 100 mM citrate buffer (pH 3.0) containing 0.04M sodium chloride. The solution is then neutralized with 1M Tris chloride (pH 80 and dialyzed against phosphate buffered saline (PBS). If necessary, the antibody may be concentrated further.

### EXAMPLE 5

### Assay for Neutralization of Growth Factor Activity

The following protocol provides an assay to determine whether an antibody substance neutralizes one or more PDGF/VEGF growth factors by preventing the growth factor(s) from stimulating phosphorylation of its receptor.

Cells such as NIH 3T3 cells are transformed or transfected with a cDNA encoding a PDGF/VEGF receptor, such as VEGFR-3, and cultured under conditions where the encoded receptor is expressed on the surface of the cells. Transfected cells are cultured with either 1) plain growth medium; 2) growth medium supplemented with 50 ng/ml of one or more ligands for the recombinant receptor, such as fully processed VEGF-C and/or VEGF-D, which are ligands for VEGFR-3; 3) growth medium supplemented with 50 ng/ml of growth factor that does not bind the recombinant receptor (e.g., VEGF-A in the case of VEGFR-3), to serve as a control; or any of (1), (2), or (3) that is first pre-incubated with varying concentrations of the antibody substance to be tested, such as an antibody that is bispecific for VEGF-C and VEGF-D.

After culturing with the culture mediums described above in the presence or absence of the antibody substance, the cells are lysed, immunoprecipitated using anti-receptor (e.g., anti-VEGFR-3) antiserum, and analyzed by Western blotting using anti-phosphotyrosine antibodies. Cells stimulated with the appropriate growth factor ligand (VEGF-C/D) stimulate VEGFR-3 autophosphorylation, which is detected with the anti-phosphotyrosine antibodies. Antibody substances that reduce or eliminate the ligand-mediated stimulation of receptor phosphorylation (e.g., in a dose-dependent manner) are considered neutralizing antibodies.

### EXAMPLE 6

### Assay for PDGF/VEGF Bispecific Antibody Blockade of Receptor Signaling

To determine if a PDGF/VEGF bispecific antibody substance blocks activation of receptor(s) by the targeted growth factors, the antibody substance is tested using bioassays of receptor binding and cross-linking.

The bioassay uses Ba/F3 pre-B cells which are transfected with a plasmid construct encoding at least one chimeric receptor consisting of the extracellular domain of either VEGFR-1, VEGFR-2, VEGFR-3, PDGFRα or PDGFRβ fused to the cytoplasmic domain of the erythropoietin (EPO) receptor (Stacker, et al., J. Biol. Chem. 274:34884-34892, 1999; Achen, et al., Eur. J. Biochem. 267:2505-2515, 2000). These cells are routinely passaged in interleukin-3 (IL-3) and will die in the absence of IL-3. However, if signaling is induced from the cytoplasmic domain of the chimeric receptors, these cells survive and proliferate in the absence of IL-3. Such signaling is induced by ligands which bind and cross-link the VEGFR-1, VEGFR-2, VEGFR-3, PDGFRα or PDGFRβ extracellular domains of the chimeric receptors. Therefore, binding of growth factor to the VEGFR-1, VEGFR-2, VEGFR-3, PDGFRα or PDGFRβ extracellular domains causes the cells to survive and proliferate in the absence of IL-3. Addition of antibodies which cause cell death (presumably by blocking the binding of growth factor to the extracellular domains) will cause cell death in the absence of IL-3 are scored as inhibitors useful for practicing methods of the invention. An alternative Ba/F3 cell line which expresses a chimeric receptor containing the extracellular domain of the Tie2 receptor, which does not bind VEGF family members, is not induced by PDGF/VEGF growth factors to proliferate and is used, in the presence of IL-3, as a control to test for non-specific effects of potential inhibitors.

Antibodies specific for VEGF-C/VEGF-D are expected to block signaling mediated by these growth factors through both the VEGFR-2 and VEGFR-3 receptor in these transfected cells, but not affect signaling of VEGFR-1 expressing cells treated with a VEGFR-1 ligand. Likewise, antibodies specific for VEGF-B/PlGF are expected to completely block these factors from causing signaling through the VEGFR-1 receptor and not VEGFR-2 or VEGFR-3. VEGF-A/VEGF-B-specific antibodies are screened for the ability to inhibit these factors from activating VEGFR-1. PDGF-C/PDGF-D antibodies are screened for the ability to interfere with these factors from signaling through the PDGFRα and PDGFRβ heterodimers or homodimers. Other permutations will be apparent from the known receptor binding profile of each PDGF/VEGF protein.

### EXAMPLE 7

### Angiogenesis Assays

There continues to be a long-felt need for additional agents that inhibit angiogenesis (e.g., to inhibit growth of tumors). Moreover, various angiogenesis inhibitors may work in concert through the same or different receptors, and on different portions of the circulatory system (e.g., arteries or veins or capillaries; vascular or lymphatic). Angiogenesis assays are employed to measure the effects of antibody substances specific for more than one PDGF/VEGF family member on angiogenic processes, alone or in combination with other angiogenic and anti-angiogenic factors, to determine preferred combination therapy involving bispecific antibodies and other modulators. Exemplary procedures include the following.

A. In Vitro Assays for Angiogenesis

1. Sprouting Assay

HMVEC cells (passage 5-9) are grown to confluency on collagen coated beads (Pharmacia) for 5-7 days. The beads are plated in a gel matrix containing 5.5 mg/ml fibronectin (Sigma), 2 units/ml thrombin (Sigma), DMEM/2% fetal bovine serum (FBS) and the following test and control proteins: 20 ng/ml of one or more PDGF/VEGF growth factors, growth factors with monoclonal antibodies specific for individual growth factors, or growth factors plus antibody substance that recognizes the growth factors, and several combinations of angiogenic factors and Fc fusion proteins. Serum free media supplemented with test and control proteins is added to the gel matrix every 2 days and the number of endothelial cell sprouts exceeding bead length are counted and evaluated. Antibody substances of the invention inhibit sprouting caused by multiple growth factors to a greater degree compared to control antibody and compared to monoclonal antibodies that recognizes only a single growth factor.

2. Migration Assay

A transwell migration assay as described below may also be used to determine the effects the antibody substance of the invention has on the interactions of growth factors, e.g., VEGF-C/VEGF-D, VEGF-A/VEGF-E, etc., with target cells. By way of example, the effects of VEGF-C and VEGF-D on migration of VEGFR-2 or VEGFR-3 expressing endothelial cells are assayed in response to a VEGF-C/VEGF-D bispecific antibody, or a VEGF-C or VEGF-D monoclonal antibody, or a control antibody. A decrease in cellular migration due to the presence of bispecific VEGF-C/VEGF-D antibody to inhibit VEGF-C or VEGF-D cellular stimulation indicates that the bispecific antibody of the invention is useful for inhibiting angiogenesis.

This assay may be carried out with cells that naturally express either VEGFR-3 or VEGFR-2, e.g., bovine endothelial cells which preferentially express VEGFR-2. Selection of naturally occurring or transiently expressing cells displaying a specific receptor depends on the bispecific antibody of the invention to be tested.

For example, human microvascular endothelial cells (HMVEC) express VEGFR-3, and such cells can be used to investigate the effect of bispecific antibody on such cells. Since VEGF/VEGFR interactions are thought to play a role in migration of cells, a cell migration assay using HMVEC or other suitable cells can be used to demonstrate stimulatory or inhibitory effects of bispecific antibody molecules.

Using a modified Boyden chamber assay, polycarbonate filter wells (Transwell, Costar, 8 micrometer pore) are coated with 50 μg/ml fibronectin (Sigma), 0.1% gelatin in PBS for 30 minutes at room temperature, followed by equilibration into DMEM/0.1% BSA at 37° C. for 1 hour. HMVEC (passage 4-9, 1×10⁵ cells) naturally expressing VEGFR-3 receptors or endothelial cell lines recombinantly expressing VEGFR-3 and/or VEGFR-2 are plated in the upper chamber of the filter well and allowed to migrate to the undersides of the filters, toward the bottom chamber of the well, which contains serum-free media supplemented with either prepro-VEGF-C, enzymatically processed VEGF-C, processed VEGF-D, or combinations thereof in the presence of varying concentrations of VEGF-C/VEGF-D bispecific antibody and VEGFR-3-Fc protein. After 5 hours, cells adhering to the top of the transwell are removed with a cotton swab, and the cells that migrate to the underside of the filter are fixed and stained. For quantification of cell numbers, 6 randomly selected 400× microscope fields are counted per filter.

In another variation, the migration assay described above is carried out using porcine aortic endothelial cells (PAEC) stably transfected with constructs such as those described previously, to express VEGFR-2, VEGFR-3, or both VEGFR-2 and VEGFR-3 (i.e., PAE/VEGFR-2, PAE/VEGFR-3, or PAE/VEGFR-2/VEGFR-3). PAEC are transfected using the method described in Soker et al. (Cell 92:735-745. 1998). Transfected PAEC (1.5×10⁴ cells in serum free F12 media supplemented with 0.1% BSA) are plated in the upper wells of a Boyden chamber prepared with fibronectin as described above. Increasing concentrations of VEGF-C and VEGF-D are added to the wells of the lower chamber to induce migration of the endothelial cells. After 4 hrs, the number of cells migrating through the filter is quantitated by phase microscopy.

An inhibition of VEGF-C and VEGF-D mediated cell-migration as a result of addition of the bispecific antibody indicates that the antibody is a useful tool for inhibiting lymphangiogenesis at the site of tumor or other aberrant lymph migration. VEGF-C/VEGF-D bispecific antibody substances of the invention are expected to inhibit migration induced by VEGF-C and VEGF-D greater than monoclonal antibodies to either growth factor.

An additional migration assay is performed wherein a solution containing growth factors (e.g., VEGF-A and VEGF-E) with (experimental) or without (control) a bispecific antibody capable of binding those growth factors, or with a mono-specific antibody, is placed in a well made in collagen gel and used to stimulate the migration of bovine capillary endothelial (BCE) cells in the three-dimensional collagen gel as follows. A further control comprising neither growth factor ligand or bispecific antibody may also be employed, as may a control with just bispecific antibody. Varied amounts of bispecific antibody may be placed in the wells to obtain more precise binding data.

BCE cells (Folkman et al., Proc. Natl. Acad. Sci. USA, 76:5217-5221, 1979) are cultured as described in Pertovaara et al. (J. Biol. Chem., 269:6271-74, 1994). These or other cells employed may be transformed with growth factor receptor if not already expressed. For testing of VEGF-A/VEGF-E bispecific antibody, cells are transformed both VEGFR-2. The collagen gels are prepared by mixing type I collagen stock solution (5 mg/ml in 1 mM HCl) with an equal volume of 2×MEM and 2 volumes of MEM containing 10% newborn calf serum to give a final collagen concentration of 1.25 mg/ml. The tissue culture plates (5 cm diameter) are coated with about 1 mm thick layer of the solution, which is allowed to polymerize at 37° C. BCE cells were seeded on top of this layer. For the migration assays, the cells are allowed to attach inside a plastic ring (1 cm diameter) placed on top of the first collagen layer. After 30 minutes, the ring is removed and unattached cells are rinsed away. A second layer of collagen and a layer of growth medium (5% newborn calf serum (NCS)), solidified by 0.75% low melting point agar (FMC BioProducts, Rockland, Me.), are added. A well (3 mm diameter) is punched through all the layers on both sides of the cell spot at a distance of 4 mm, and the experimental or control media are pipetted daily into the wells. Photomicrographs of the cells migrating out from the spot edge are taken after six days through an Olympus CK 2 inverted microscope equipped with phase-contrast optics. The migrating cells are counted after nuclear staining with the fluorescent dye bisbenzimide (1 mg/ml, Hoechst 33258, Sigma).

The number of cells migrating at different distances from the original area of attachment towards wells containing experimental or control solutions are determined 6 days after addition of the media. The number of cells migrating out from the original ring of attachment is counted in five adjacent 0.5 mm×0.5 mm squares using a microscope ocular lens grid and 10× magnification with a fluorescence microscope. Cells migrating further than 0.5 mm are counted in a similar way by moving the grid in 0.5 mm steps. The experiments are carried out twice with similar results. Daily addition of 1 ng of FGF2 into the wells may be employed as a positive control for cell migration. A decrease in migration when both growth factor(s) and bispecific antibody is employed relative to when growth factor alone is employed, or growth factors plus monoclonal antibody, indicates that the bispecific antibody effectively blocks stimulation by both growth factors.

B. In Vivo Assays for Angiogenesis or Lymphangiogenesis

1. Chorioallantoic Membrane (CAM) Assay

Three-day old fertilized white Leghorn eggs are cracked, and chicken embryos with intact yolks are carefully placed in 20×100 mm plastic Petri dishes. After six days of incubation in 3% CO₂ at 37° C., a disk of methylcellulose containing at least two PDGF/VEGF molecules, such as VEGF-A, VEGF-B, VEGF-C VEGF-D, VEGF-E, PDGF-C or PDGF-D and control monoclonal antibody or a bispecific antibody substance, and/or soluble VEGFR complexes, dried on a nylon mesh (3×3 mm) is implanted on the CAM of individual embryos, to determine the influence of bispecific antibody on vascular development, and potential uses thereof to inhibit vascular formation. The nylon mesh disks are made by desiccation of 10 microliters of 0.45% methylcellulose (in H₂O). After 4-5 days of incubation, embryos and CAMs are examined for the formation of new blood vessels and lymphatic vessels in the field of the implanted disks by a stereoscope. Disks of methylcellulose containing PBS are used as negative controls. Antibodies that recognize both blood and lymphatic vessel cell surface molecules are used to further characterize the vessels.

2. Corneal Assay

Corneal micropockets are created with a modified von Graefe cataract knife in both eyes of male 5- to 6-week-old C57BL6/J mice. A micropellet (0.35×0.35 mm) of sucrose aluminum sulfate (Bukh Meditec, Copenhagen, Denmark) coated with hydron polymer type NCC (IFN Science, New Brunswick, N.J.) containing various concentrations of two or more PDGF/VEGF molecules alone or in combination with: i) factors known to modulate vessel growth (e.g., 80 ng of FGF-2); ii) monoclonal antibody specific for one of the growth factors; or iii) a bispecific antibody composition. The pellet is positioned 0.6-0.8 mm from the limbus. After implantation, erythromycin/ophthamic ointment is applied to the eyes. Eyes are examined by a slit-lamp biomicroscope over a course of 3-12 days. Vessel length and clock-hours of circumferential neovascularization and lymphangiogenesis are measured. Furthermore, eyes are cut into sections and are immunostained for blood vessel and/or lymphatic markers LYVE-1 (Prevo et al., J. Biol. Chem. 276: 19420-19430, 2001), podoplanin (Breiteneder-Geleff et al., Am. J. Pathol. 154: 385-94, 1999) and VEGFR-3 to further characterize affected vessels. Bispecific antibody substances of the invention inhibit vessel growth relative to monoclonal or control antibodies.

It will be apparent that the numerous assays described above, can be performed with different permutations of growth factors, growth factor receptors (recombinantly introduced into cells), monospecific, and bispecific antibody substances of the invention.

### EXAMPLE 8

### Assay for Growth Factor Mediated Tumor Growth and Metastasis

For some cancers, overexpression of VEGF-C in cancer cells leads to increased tumor size and lymphangiogenesis, while overexpression of VEGF-A has a greater angiogenic effect. See, e.g., WO 02/060959, incorporated herein by reference. Other PDGF/VEGF factors have been shown to, or may be expected to, have similar effects. In vivo, different cancers may have varying expression patterns of any number of these growth factors producing varied tumorigenic effects.

To assess the effects of blockade of one or more growth factors on tumor progression, tumor cells such as MCF-7 human breast carcinoma cells can be, recombinantly modified to over-express one or more of these growth factors, and then implanted into laboratory animals, to demonstrate and model the in vivo role of the PDGF/VEGF family of growth factors in tumorigenesis. For example, such cells are orthotopically implanted into SCID mice, and the extent of tumor growth, tumor spread, and tumor angiogenesis and angiogenesis can be measured. The mice are administered monoclonal antibodies against one growth factor, or antibody substances of the invention that bind and inhibit multiple growth factors, to demonstrate the ability of antibody substances of the invention to inhibit tumor growth or metastases or progression.

For example, to prepare tumor cells expressing a PDGF/VEGF family member, the cDNAs coding for at least two of the human VEGF-A, VEGF-B, VEGF-C, VEGF-D, VEGF-E, PDGF-C or PDGF-D are introduced into the pEBS7 plasmid (Peterson and Legerski, Gene, 107: 279-84, 1991) and subsequently transfected into a tumor cell line. In one aspect, the MCF-7S1 subclone of the human MCF-7 breast carcinoma cell line is transfected with the PDGF/VEGF plasmid DNA by electroporation, and stable cell pools are selected and cultured as previously described (Egeblad and Jaattela, Int J Cancer, 86: 617-25, 2000).

In order to detect the secreted protein in the transfected cell culture media, the cells are metabolically labeled in methionine and cysteine free MEM (Gibco) supplemented with 100 μCi/ml [³⁵S]-methionine and [³⁵S]-cysteine (Redivue Pro-Mix, Amersham Pharmacia Biotech). The labeled growth factors are immunoprecipitated from the conditioned medium using antibodies against the respective growth factors, e.g. VEGF-C (Joukov, et al., EMBO J, 16: 3898-911, 1997) or VEGF-A (MAB293, R & D Systems). The immunocomplexes are precipitated using protein A sepharose (Amersham Pharmacia Biotech), washed twice in 0.5% BSA, 0.02% Tween 20 in PBS and once in PBS and analyzed in SDS-PAGE under reducing conditions.

PDGF/VEGF transfected MCF7 cells (20,000/well) are plated in quadruplicate in 24-wells, trypsinized on replicate plates after 1, 4, 6, or 8 days and counted using a hemocytometer. Fresh medium is provided after 4 and 6 days. For the tumorigenesis assay, sub-confluent cultures are harvested by trypsination, washed twice and 10⁷ cells in PBS are inoculated into the fat pads of the second (axillar) mammary gland of ovariectomized SCID mice, carrying subcutaneous 60-day slow-release pellets containing 0.72 mg 17β-estradiol (Innovative Research of America). The ovariectomy and implantation of the pellets are performed 4-8 days before tumor cell inoculation. The animals are treated with antibody substances of the invention, or monoclonal antibodies, or control substances as described below, for various lengths of time. Tumor length and width are measured twice weekly in a blinded manner, and the tumor volume is calculated as the length×width×depth×0.5, assuming that the tumor is a hemi-ellipsoid and the depth is the same as the width (Benz et al., Breast Cancer Res Treat, 24: 85-95, 1993).

The tumors are excised, fixed in 4% paraformaldehyde (pH 7.0) for 24 hours, and embedded in paraffin. Sections (7 μm) are immunostained with monoclonal antibodies against several cellular markers including: PECAM-1 (Pharmingen), an endothelial antigen primarily expressed in blood vessels and only weakly in lymphatic vessels; VEGFR-3 (Kubo et al., Blood, 96: 546-553, 2000); PCNA (Zymed Laboratories) to detect actively proliferating cells; polyclonal antibodies against LYVE-1 to detect lymph vessels (Banerji et al., J Cell Biol, 144: 789-801, 1999); VEGF-C (Joukov et al., EMBO J, 16: 3898-911, 1997); or, laminin, according to published protocols (Partanen et al., Cancer, 86: 2406-12, 1999). The average of the number of the PECAM-1 positive vessels is determined from three areas (60× magnification) of the highest vascular density (vascular hot spots) in a section. All histological analysis is performed using blinded tumor samples.

Animals given MCF7 cells overexpressing PDGF/VEGF growth factors are administered control antibody, bispecific antibody substances, or monoclonal antibodies specific for only one growth factor, in order to measure the reduction or inhibition of tumor growth. These antibodies are administered at various timepoints after induction of tumor cells, e.g., on day 7, day 10, or day 14 post induction, or may be administered daily for 1 or 2 weeks post tumor induction. Regimens are determined according to routine experimentation. The tumors are then removed and stained as described above to determine the effects of bispecific antibody substance on tumor growth and tumor metastasis.

It is contemplated that this assay is performed using MCF7 cells or other pre-cancer cell line or primary tumor that overexpress any combination of PDGF/VEGF proteins, including VEGF-A, VEGF-B, VEGF-C, VEGF-D, VEGF-E, PlGF, PDGF-C or PDGF-D, and the effects of bispecific antibody substances for any of two the growth factors contemplated is assessed as above. Bispecific antibody substances of the invention inhibit tumor growth and tumor spread relative to monoclonal or control antibodies.

### EXAMPLE 9

### Inhibition of VEGF-C Binding to VEGFR-2 or VEGFR-3 by Bispecific VEGF-C/VEGF-D Antibody

The redundancy of binding between the VEGF-C and VEGF-D molecules indicates that the bispecific VEGF-C/VEGF-D antibody inhibits both VEGF-C and -D ligand binding to the VEGFR-2 and VEGFR-3 receptors, providing a therapeutic method for abolishing VEGFR-3 receptor signaling and reduced VEGFR-2 signaling. The following examples are designed to provide proof of this therapeutic concept.

A. In Vitro Cell-Free Assay

To demonstrate the inhibitory effects of bispecific VEGF-C/VEGF-D antibody substance against binding to the VEGFR-2 or VEGFR-3, microtiter plates are pre-coated with 1 μg/ml of VEGFR-3 or VEGFR-2. A mixture of VEGF-C and VEGF-D protein is conjugated to a label, e.g. a biotin molecule, and incubated with bispecific VEGF-C/VEGF-D antibody, VEGFR-2 Fc or VEGFR-3-Fc, a monoclonal antibody specific for only one growth factor, or control protein at various molar ratios. After blocking the coated plates with 1% BSA/PBS-Tween, the aforementioned mixtures are applied on the microtiter plates overnight at 4° C. Thereafter, the plates are washed with PBS-T, and 1:1000 of avidin-HRP is added. Bound VEGF-C and VEGF-D protein is detected by addition of the ABTS substrate (KPL). The bound labeled growth factors are is analyzed in the presence and absence of the bispecific VEGF-C/VEGF-D antibody substance or soluble VEGFRs or monoclonal antibodies and the percent inhibition of binding assessed.

Inhibition of ligand binding to VEGFR-2/R-3 by the bispecific VEGF-C/VEGF-D antibody that is comparable to that of the soluble receptors indicates that the bispecific VEGF-C/VEGF-D antibody is a potent inhibitor of both VEGF-C and VEGF-D activity mediating the VEGFR related angiogenesis and lymphangiogenesis, and provides a method for modulating aberrant angiogenesis and lymphangiogenesis. The antibody substances of the invention are expected to inhibit the amount of VEGF-C+VEGF-D bound to the plates better than a monoclonal antibody specific for either growth factor.

B. In Vitro Cell-Based Assay

VEGF-C and VEGF-D are used as described above to contact cells that naturally or recombinantly-express VEGFR-2 and VEGFR-3 on their surface. By way of example, 293EBNA or 293T cells recombinantly modified to transiently or stably express VEGFR-2 and VEGFR-3 as described above are employed. Several native endothelial cell types express both receptors and can also be employed, including but not limited to, human microvascular endothelial cells (HMEC) and human cutaneous fat pad microvascular cells (HUCEC).

Signaling through VEGFRs is detected by evidence of tyrosine phosphorylation of the intracellular tyrosine kinase domain. To assess autophosphorylation of VEGFR-3, 293T or 293EBNA human embryonic kidney cells grown in Dulbecco's modified Eagle's medium (DMEM) supplemented with 10% fetal calf serum (GIBCO BRL), glutamine and antibiotics, are transfected using the FUGENE TM6 transfection reagent (Roche Molecular Biochemicals) with plasmid DNAs encoding the receptor constructs (VEGFR-3 or VEGFR-3-myc tag or an empty pcDNA3.1z+ vector (Invitrogen). For stimulation assay, the 293EBNA cell monolayers are starved overnight (36 hours after transfection) in serum-free medium containing 0.2% BSA. The 293EBNA cells are then stimulated with 300 ng/ml of recombinant VEGF-C ΔNΔC (Joukov et al., EMBO J. 16:3898-3911. 1997) and VEGF-D ΔNΔC for 10 min at 37° C., in the presence or absence of bispecific VEGF-C/VEGF-D antibody or monospecific monoclonal antibody to determine inhibition of ligand/VEGFR-3 binding. The cells are then washed twice with cold phosphate buffered saline (PBS) containing 2 mM vanadate and 2 mM phenylmethylsulfonyl fluoride (PMSF), and lysed into PLCLB buffer (150 mM NaCl, 5% glycerol, 1% Triton X-100, 1.5 M MgCl2, and 50 mM Hepes, pH 7.5) containing 2 mM Vanadate, 2 mM PMSF, 0.07 U/ml Aprotinin, and 4 mg/ml leupeptin. The lysates are centrifuged for 10 min at 19 000 g, and incubated with the supernatants for 2 hours on ice with 2 μg/ml of monoclonal anti-VEGFR-3 antibodies (9D9f9) (Jussila et al., Cancer Res. 58:1599-1604. 1998), or alternatively with antibodies against the specific tag epitopes, for example, 5 μg/ml anti-Myc antibodies (BabCO). The Immunocomplexes are incubated with protein A sepharose (Pharmacia) for 45 minutes with rotation at 4° C. and the sepharose beads washed three times with cold PLCLB buffer (2 mM vanadate, 2 mM PMSF). The bound polypeptides are separated by 7.5% SDS-PAGE and transferred to a Protran nitrocellulose filter (Schleicher & Schuell) using semi-dry transfer apparatus. After blocking with 5% BSA in TBS-T buffer (10 mM Tris pH 7.5, 150 mM NaCl, 0.1% Tween 20), the filters are stained with the phosphotyrosine-specific primary antibodies (Upstate Biotechnology), followed by biotinylated goat-anti-mouse immunoglobulins (Dako) and Biotin-Streptavidin HRP complex (Amersham) Phosphotyrosine-specific bands are visualized by enhanced chemiluminescence (ECL). To analyze the samples for the presence of VEGFR-3, the filters are stripped for 30 min at +55° C. in 100 mM 2-mercaptoethanol, 2% SDS, 62.5 mM Tris-HCl pH 6.7 with occasional agitation, and stained with anti-receptor antibodies and HRP conjugated rabbit-anti-mouse immunoglobulins (Dako) for antigen detection. Reduced VEGFR-3 autophosphorylation is indicative of successful bispecific VEGF-C/VEGF-D antibody mediated inhibition of VEGF-C/VEGFR3 and VEGF-D/VEGFR3 binding. It is contemplated that the same procedure is carried out with VEGFR-2 transfected cells.

### EXAMPLE 10

### Administration of PDGF-C/PDGF-D Bispecific Antibody

PDGF/VEGF growth factors are intimately involved with many functions of angiogenesis and lymphangiogenesis and endothelial cell growth. The influence of bispecific antibody substances on growth factor functions in vivo is investigated using the following methods, as exemplified by PDGF-C/PDGF-D bispecific antibody substances.

Overexpression of the PDGFs has been observed in several pathological conditions, including malignancies, atherosclerosis, and fibroproliferative diseases. Both PDGF-AA and PDGF-CC bind PDGFR-α, but only PDGF-CC potently stimulates angiogenesis in mouse cornea pocket and chick chorioallanoic membrane (CAM) assays (Cao, et al., FASEB. J 16:1575-83, 2002). PDGF-CC also promotes wound healing by stimulating tissue vascularization (Gilbertson et al., J. Biol. Chem. 10:10, 2001). Both PDGF-C and PDGF-D have been implicated as potent angiogenic factors (Li et al., Oncogene 22:1501-10, 2003), and have been observed to be upregulated in brain tumors such as glioblastomas (Lokker et al., Cancer Res. 62:3729-35, 2002), which also express PDGFRs.

PDGF-D is a potent transforming growth factor for cultured fibroblast cells, inducing increased proliferation, anchorage-independent growth in soft agar, the ability to induce tumors in nude mice, and upregulation of vascular endothelial growth factor. PDGF-D appears to be a factor capable of inducing cellular transformation and promoting tumor growth by accelerating the proliferation rate of the tumor cells, and by stimulation of tumor neovascularization. An agent that neutralized more than one of the PDGF molecules at the same time would provide increased therapeutic potential to treat aberrant cell proliferation mediated by PDGFs. For example, bispecific antibodies that react with both PDGF-C and PDGF-D would simultaneously neutralize PDGF-D and PDGF-C action in many conditions relating to undesired angiogenesis and tissue growth, for example in blocking tumor growth, and in fibrotic diseases. Bispecific antibodies that cross-react with both PDGF-C and PDGF-D may also prevent or inhibit processing of these proteins to their active form, thereby neutralizing and/or blocking activation of the proteins.

The following procedures were performed to identify a population of antibodies in a polyclonal rabbit antiserum raised against full-length PDGF-D that can cross-react with PDGF-C. To isolate antibodies that bind PDGF-C from anti-PDGF-D rabbit antiserum, mouse full-length PDGF-D cDNA (Genbank Accession No. NP_{—}064355) was cloned into pcDNA3.1/zeo(+) mammalian expression vector (Invitrogen). The core-domain of mouse PDGF-C coding sequence downstream of the predicted proteolytic processing site (corresponding to amino acids 243-345 of SEQ ID NO: 12) was amplified by PCR, using the primers:

(forward) (SEQ ID NO: 25)

5′-CGCGGATCCGAAGAGGTAAAACTCTACAGCTGC,
and

(reverse) (SEQ ID NO: 26)

5′-GGAATTCCCCCTCCTGCGTTTCCTCTACA.

The PCR product was cloned in frame with the Ig K-chain leader sequence to direct the truncated protein for secretion using a modified pSecTag2A vector (Invitrogen). Following the core-domain PDGF-C coding sequence, a C-terminal c-myc and Hisx6 tags were obtained from the vector. The insert from this construct was cleaved and cloned into pCDNA3.1 vector. All constructs were verified by sequencing analysis.

COS-1 cells were maintained in DMEM containing 10% fetal bovine serum and penicillin/streptomycin. The COS-1 cells were seeded in 6-well plates and transfected with 2.0 μg of expression plasmid encoding either full-length mouse PDGF-D, or core-domain mouse PDGF-C.

Twenty-four hours post-transfection, the cells were washed and serum-free medium was added. After an overnight incubation, serum-free media were collected. To detect cross-reactivity of PDGF-C protein with PDGF-D antibody, 900 μl of serum-free medium containing core domain PDGF-CC protein, and 100 μl of medium containing full-length PDGF-D protein were TCA-precipitated, and subjected to SDS-PAGE analysis under reducing conditions in a 12% SDS-PAGE gel. The gel was blotted on a PVDF membrane, and the blot blocked with 5% milk/PBS with 0.1% BSA at 4° for 1 hour. The reactivity of the PDGF-D antibody with PDGF-C protein was detected using affinity purified Ig-fractions from a rabbit antiserum raised against full-length human PDGF-D.

The PDGF-C and PDGF-D specific antibody within the antiserum was isolated from the antiserum by affinity purification of Ig fractions. The antiserum was passed over a column (Sepharose 4B or agarose) of immobilized recombinant PDGF-C (core domain or full-length protein). The bound antibody was released by eluting it with 100 mM citrate buffer (pH 3.0) containing 0.04M sodium chloride. The solution was then neutralized with 1M Tris chloride (pH 8.0) and dialyzed against phosphate buffered saline (PBS). The antibody may be concentrated further as necessary.

To assess the specificity of the isolated antibody, 2 μg Ig/ml was used in an overnight incubation at 4° C. PDGF-C bound antibodies were observed using ECL+reagent (Amersham) and visualized using a CCD camera (Fuji). The same blot was stripped and PDGF-C protein was detected using affinity purified Ig-fraction raised against human core-domain PDGF-C using 2 μg Ig/ml.

Immunoblotting analysis showed that antibodies of the anti-PDGF-D polyclonal rabbit antiserum displayed strong reactivity with full-length PDGF-D protein. In addition, the anti-PDGF-D polyclonal serum detected PDGF-C in the supernatant. The reactivity was less strong with core domain PDGF-C, as approximately similar intensities of the bands were obtained using a 9-fold concentration of conditioned medium from the PDGF-C core domain transfected cells. As a control, the same immunoblot was probed with a PDGF-C antibody (Li et al Nat. Cell Biol. 2:302-309 2000), showing strong reactivity with PDGF-C only. These results demonstrated that a sub-population of antibodies generated against full-length PDGF-D are able to cross-react with PDGF-C.

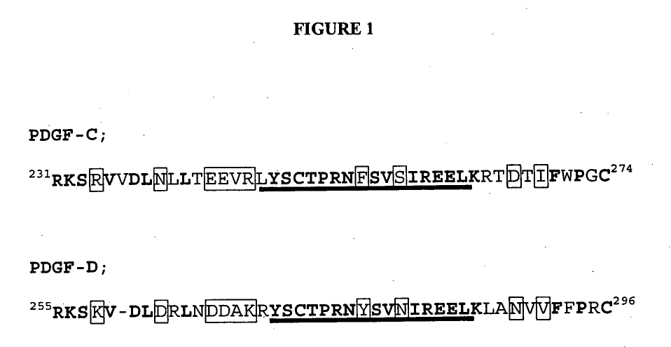

Bispecific anti-PDGF-C/PDGF-D antibodies are also generated using an antigen which contains sequence similarity between the two PDGF proteins. The amino acid sequences of human PDGF-C and human PDGF-D are aligned using conventional algorithms to identify regions of sequence similarity. A sequence of at least 6 consecutive, identical amino acids is preferred, with 7, 8, 9, 10, 11, 12, 13, 14, 15, or more being highly preferred. Mismatches within the selected peptide sequences preferably are still conservative substitution-type mismatches such that they are unlikely to interfere with cross-reactivity, e.g. where an acidic amino acid such as aspartic acid (D) in one protein aligns with glutamic acid (E) in the other; or where the side chains for the amino acids are not going to interfere with antibody cross-reactivity. Exemplary peptides include amino acids 231-274 of PDGF-C (SEQ ID NO: 27) which correspond by way of alignment with amino acids 255-296 of PDGF-D (SEQ ID NO: 28) (FIG. 1), or fragments of 5 or more amino acids from said peptides:

| | |
|---|---|
| ²³¹RKSRVVDLNLLTEEVRLYSCTPRNFSVSIRE | (SEQ ID NO: 27) |
| ELKRTDTIFWPGC²⁷⁴PDGF-C | |
| ²⁵⁵RKSKV-DLDRLNDDA KRYSCTPRNYSVNIR | (SEQ ID NO: 28) |
| EELKLANVVFFPRC²⁹⁶PDGF-D | |

The selected peptides are used to immunize a laboratory animal using standard techniques to generate an immune response and isolated antibodies from immunized animals as described in Example 2 above.

A comparison of the localization of the preferred shared epitope between PDGF-C and PDGF-D (FIG. 1) to the corresponding region in the 3-dimensional structure of the highly related PDGF-BB molecule (Oefner et al., EMBO J. 11:3921-3926, 1992), indicates this region of similarity in PDGF-BB is located on the surface of the growth factor and easily accessible to antibody binding. Antibody binding to this region would mimic the N-terminal CUB domain present in latent PDGF-C and PDGF-D, suggesting that antibody binding to this region would neutralize the biological action of PDGF-C, PDGF-D and PDGF-BB, thereby generating a three-way crossreactive antibody that antagonizes a significant amount of PDGF activity.

The PDGF-C/D bispecific antibody is assessed for its ability to neutralize PDGF activity using the neutralization assay as set out above. The PDGF-C/D bispecific antibody is also assessed for inhibition of proteolytic processing of PDGF-C or PDGF-D by proteases from the inactive full-length protein into the active form. Previous studies have demonstrated that. PDGF-C cleavage occurs extra-cellularly and is mediated primarily by serine proteases (U.S. Pat. Publ. No. 2003/0211994). To measure the inhibition of PDGF-C or -D processing by a PDGF-C/D bispecific antibody of the invention, full-length PDGF-C/D protein expressed in medium from transfected cells is incubated with proteolytic enzymes (e.g., TPA is useful to measure PDGF-C proteolysis), with or without bispecific antibody, to determine if PDGF-C or PDGF-D proteolysis is prevented.

Additionally, a protease inhibitor analysis may be conducted as described in U.S. Pat. Publ. No. 2003/0211994. The inhibition of protein cleavage by the bispecific antibody is compared to various protease inhibitors, including, for example, inhibitors of serine proteases such as AEBSF, leupeptin, and aprotinin, or other protease inhibitors such as bestatin, pepstatin A, E64, EDTA and phosphoramidon. The bispecific antibody or protease inhibitor(s) are pre-incubated with 0.9 ml AG1523 serum free media, containing endogenous serum proteases, at room temperature for 30 minutes, then incubated with 0.2 ml of recombinant full-length PDGF-C (Sf9 serum-free medium) at 37° C. overnight. TCA-precipitated proteins are subjected to SDS-page under reducing conditions and then immunoblotted.

Recombinant PDGF-C is detected using an anti-His₆ epitope monoclonal antibody (C-terminal) (InVitrogen). Similar assays are performed to assess the inhibition of PDGF-D cleavage by PDGF-C/D bispecific antibodies. A decrease in the amount of PDGF-C or PDGF-D cleavage product as measured by SDS-gel indicates that the bispecific antibody acts as an inhibitor of PDGF-C/D cleavage and subsequently prevents protein activation.

The ability of PDGF-C/PDGF-D bispecific antibodies to treat or reduce fibrosis is measured using an experimental model of fibrotic disease, including those described in Murata et al (J Surg. Res. 114:64-71, 2003), which describes the effects of Rho-kinase inhibition in hepatic cells in a carbon tetrachloride (CCl₄)-induced rat liver fibrosis model, Schanstra et al (J Clin Invest. 110:371-9, 2002), which assesses the effects of bradykinin deficiency in a unilateral ureteral obstruction (UUO) induced model of renal fibrosis, Schmitt et al. (Blood 96:1342-47, 2000), which discloses a mouse model of thrombopoietin-induced myelofibrosis resulting in increased bone marrow collagen, or Ponten et al. (Am J Pathol. 163:673-82, 2003), which teaches that transgenic animals overexpressing PDGF-C develop cardiomypathy and cardiac fibrosis. PDGF antibodies are administered to animals either before or after induction of fibrosis to determine the ability of the PDGF bispecific antibodies to ameliorate symptoms of fibrosis. Doses and timing of bispecific antibody administration is determined using standard dosing studies well known in the art.

Transgenic mice expressing either full length PDGF-C or PDGF-D are useful as models for fibrosis and can be used to test the neutralizing ability of PDGF-C/D bispecific antibodies. PDGF-C transgenic mice are described in Ponten et al (supra) and in International Patent Publication WO 01/72132. PDGF-D transgenics are described in U.S. Patent Publication No. 2003/0073637.

In addition, the ability of the PDGF-C/PDGF-D bispecific antibody to ameliorate tumor growth is measured using experimental animal models for neuroblastoma (Engler et al., Cancer Res. 61:2968-73, 2001) or glioblastoma (Kawakami et al., J Neurooncol. 65:15-25, 2003). Reduction of tumor size in animals receiving the bispecific antibody compared to control animals receiving control antibody or monoclonal antibody specific for only one growth factor indicates that the blockade of PDGF-C and PDGF-D signaling is an effective method for treating these central nervous system derived tumors.

### EXAMPLE 11

### Construction of Antibodies to Regions of Similarity in PDGF-C AND PDGF-D

The example above demonstrates that antibodies generated against full-length PDGF-D may cross-react with PDGF-C, thereby acting bispecifically. A preferred bispecific antibody is one that binds both PDGF-C and PDGF-D and neutralizes the activity of these growth factors. The core domain region in PDGF-D contains the cleavage site for the PDGF-DD activating enzyme. Antibodies to this core region are selected that cross-react with PDGF-C, and also block activation and act antagonistically to both PDGF-DD and PDGF-CC.

To assess if an antibody directed against the PDGF-DD core region that exhibits the greatest similarity between the PDGF-C and PDGF-D acts as a neutralizing antibody, the region was cloned, expressed and purified using techniques standard in the art, and animals were immunized with the peptide to produce polyclonal antisera.

A synthetic peptide used to generate PDGF-DD antisera was derived from the human PDGF-D sequence, amino acid 255-272, sequence CKSKVDLDRLNDDAKRYSC (SEQ ID NO: 29). The N-terminal cysteine residue is not present in the native PDGF-D amino acid sequence, but was added to increase the coupling efficiency, as described in Bergsten at al. Nature Cell Biol. 3:512-516, 2001. Antibodies were generated using well-established techniques. Briefly, rabbits were immunized with human PDGF-D 255-272 peptide emulsified in Complete Freunds Adjuvant. Following repeated booster immunizations using the same amount of antigen, PDGF-D reactivity is assayed by determining the antibody titer in small blood samples obtained from immunized animals.

Four to six weeks following immunization and booster injections, animals demonstrating strong anti-PDGF-D antibody titer are then bled through the tail vein and polyclonal sera purified using column purification techniques described previously. The antiserum was passed over a column (Sepharose 4B or agarose) (Sulpho Link, Pierce Biotechnology, Rockford, Ill.) of immobilized recombinant PDGF-D (peptide 255-272, core domain or full-length protein). The bound antibody was released by eluting it with 100 mM citrate buffer (pH 3.0) containing 0.04M sodium chloride. The solution was then neutralized with 1M Tris chloride (pH 8.0) and dialyzed against phosphate buffered saline (PBS). The antibody may be concentrated further as necessary.

Antibody neutralization assays were performed using active core domain peptide. Active core domain PDGF-DD was generated by subjecting human PDGF-D cDNA to PCR mutagenesis using the following primers 5′-TAATGGATCCGGCAGGTCA (forward) [SEQ ID NO: 30] including a flanking BamH I site and 5′-TAATCTCGAGCTCGAGGTGG (reverse) [SEQ ID NO: 31] including a flanking Xho I site. The resulting 370-bp fragment (starting at the nucleotide coding for amino acid 248 in human PDGF-D) was digested with BamH I/Xho I and cloned into a modified pSecTag2 vector (part of multiple cloning site modified by restriction cleavage of the sequence between Sfi I and Kpn I, followed by ligation of the cleaved vector). Cos-1 cells, maintained in DMEM (10% FBS, 2 mM glutamin, 100 U ml⁻¹ pencillin and 100 μg ml⁻¹ streptomycin.) were transfected with 2 μg of the pSecTag2-core PDGF-D vector, in DMEM using Lipofectamine Plus (Invitrogen Life Technologies, Carlsbad, Calif.).

After 24 hours the media was changed to serum-free medium. After an additional 24 hours the serum-free media containing recombinant active core PDGF-DD (recombinantly processed PDGF-D) was harvested and diluted 1:2, 1:10, 1:100 and 1:1000 in PBS containing 1 mg/ml of BSA, 0.9 mM CaCl₂, and 0.49 mM MgCl₂. One milliliter of each dilution of PDGF-DD conditioned medium was incubated with 10 μg of the PDGF-D-affinity-purified rabbit peptide antibody for 2 hours on ice. As a control, the 1:2 dilution of the conditioned medium was incubated with 10 μg of preimmune rabbit IgG.

The antibody-treated samples were then used in a neutralization assay to assess inhibition of PDGF-C and PDGF-D induced PDGFR-β tyrosine phosphorylation. Serum-starved porcine aortic endothelial cells (PAE), stably expressing human PDGFR-β (Heldin et al, EMBO J. 7:1387-1393, 1998; Eriksson et al., EMBO J. 11:543-550, 1992), were incubated on ice for 60 minutes with 1 ml of control-treated or anti-PDGF-D treated PDGF-D samples. The treated PAE cells were then lysed in 20 mM Tris-HCl pH 7.5, 0.5% Triton X-100, 0.5% Na deoxycholate, 5 mM EDTA, 150 mM NaCl, 200 μM orthovanadate and protease inhibitors (Complete; Roche, Mannheim, Germany). Supernatants were collected and equal amounts of protein from each lysate were immunoprecipitated (on ice) using a rabbit antiserum to PDGFR-β, followed by incubation with protein A-Sepharose. Beads were washed and boiled in 2×SDS loading buffer and subjected to 7.5% SDS-PAGE under reducing conditions. Immunoblotting was performed using a monoclonal anti-phosphotyrosine antibody (PY99, 1 μg/ml). Bound antibodies were visualized using ECL (Amersham biosciences, Piscataway, N.J., USA) as described in Bergsten at al. (supra).

Phosphotyrosine staining showed that antibodies specific for the selected core domain PDGF-D peptide neutralized PDGFR-β activation as indicated by a significant decrease in receptor tyrosine phosphorylation in cells cultured with anti-PDGF antibody-treated media. Incubation with preimmune Ig had no neutralization activity, and medium from mock-transfected cells failed to activate the receptor.

These data showed that antibodies to the epitopes corresponding to the N-terminal portion of the core domain of PDGF-D (amino acids 255-272) has PDGF-D neutralizing activity. Since the proposed activation site for latent full-length PDGF-DD is located in the N-terminal part of the peptide used to raise the anti-peptide antibodies, it is hypothesized that these antibodies block activation of latent PDGF-DD by sterically hindering the activating protease from cleaving the full-length factor.

As described above, this core domain region is very similar between PDGF-C and PDGF-D. Therefore, antibodies raised to the region corresponding to the peptide used in these experiments, or to a peptide extending N- or C-terminal of the used peptide, that cross-react with PDGF-C are predicted to prevent activation of the latent PDGF-C and neutralize the biological activity of the active factor. Furthermore, PDGF-C cross reactive antibodies are predicted to also prevent proteolytic activation of PDGF-CC and to neutralize the biological activity of core domain PDGF-CC by preventing its interaction with PDGFRα, the receptor for activated core domain PDGF-CC.

Bispecific Antibodies to PDGF-DD and PDGF-CC Activated Growth Factors

It is contemplated that antibodies specific for the activated PDGF-C and PDGF-D growth factors are useful in treatment of various disease including prevention of tumor growth and metastasis, tissue fibrosis, kidney disease, vascular diseases of the eye, and other conditions mediated by aberrant PDGF-C or PDGF-D expression.

To generate active core domain specific antibodies, any portion of the PDGF-D fragment 255-296 may be used to generate antibodies. For example, the corresponding PDGF-D core domain peptide described above, or derivatives thereof, is synthesized to include an internal or added cysteine residue. This peptide is coupled to keyhole limpet hemocyanin (KLH) using the heterobifunctional crosslinker SPDP according to the instructions supplied by the manufacturer (Pierce Biotechnology Inc). The peptide conjugate is subsequently mixed with Freunds Complete Adjuvant and used to immunize mice as described above. Additionally, the core domain of PDGF-C, amino acids 231-274 which demonstrates the greatest similarity to the PDGF-peptide is used to generate core domain bispecific antibodies. PDGF-C specific peptides useful in making core domain specific antibodies optionally comprise fragments of the 231-274 fragment, including a PDGF-C peptide comprising amino acids 231-250.

Dilutions of the anti-peptide sera are screened for reactivity with full-length PDGF-DD, core domain PDGF-DD, full-length PDGF-CC and core domain PDGF-CC by standard ELISA techniques. The proteins are coated onto the ELISA plates in 50 ul aliquots of the protein solutions (dilution in 100 mM amoniumbicarbonate buffer pH 8.0 to a final concentration of 1 ug/ml) and incubated over night. The plates are then washed in 20 mM Tris-Hcl buffer pH 8.0 containing 150 mM NaCl and 0.1% Tween-20. Serial dilutions of the PDGF-DD immune sera are then analyzed in these plates. Bound Ig is detected using enzyme-labeled secondary antibodies.

Mice producing antibodies that react with both PDGF-DD and PDGF-CC are selected for the generation of monoclonal antibodies using standard technologies. Selected monoclonal antibodies are expanded and purified Ig preparations are prepared. These Ig preparations are characterized for neutralization activity as described above by preincubating the antibodies with core domain PDGF-DD, or core domain PDGF-CC, and than applying these samples onto PAE-1 cells separately expressing PDGFRβ or PDGFRα. Neutralizing activity is recorded as a decrease in receptor activation by the antibody-bound peptides compared activation levels using control Ig preincubated samples.

To test for the ability of the monoclonal antibodies to prevent proteolytic activation of latent PDGF-DD or latent PDGF-CC, expression plasmids encoding the full-length factors and the relevant activating proteases, such as tissue plasminogen activator for latent PDGF-CC (Fredriksson et al. EMBO J. 23:3793-3802, 2004), are co-expressed in transfected. COS-1 cells. Antibody preparations of the selected monoclonal antibodies are added to the newly transfected cells, and the cells are incubated overnight. The conditioned media from the transfected cells are subsequently subjected to TCA precipitations and precipitated proteins are analyzed by SDS-PAGE under reducing conditions and immunoblotting. PDGF-C and PDGF-D chains are detected using specific antibodies using established techniques. Full-length PDGF-C or PDGF-D proteins migrate as 45 and 50 kDa species, respectively. The presence of released core domains of both factors are indicated by species migrating at 20-23 kDa.

The ability of antibody preparations to inhibit the cleavage of the full-length factors is determined by the absence or reduction in amounts of the shorter cleaved form of the PDGF-CC/PDGF-DD proteins. Cross-reactive antibodies are determined by ELISA. Neutralization assays are performed as deceribed above using PDGFR-β transfected PAE cells. Antibodies that prevent the proteolytic activation of both factors and neutralize the activity of both factors are useful in development of therapies to prevent their action in conditions where PDGF-C or PDGF-D are overexpressed.

It is further contemplated that the monoclonal antibodies described above are useful in making single chain antibodies, antibody fragments, such as Fab, Fab₂, humanized antibodies, and chimeric antibodies. Further contemplated are human monoclonal antibodies generated against the PDGF-DD polypeptide.

### EXAMPLE 12

### Screening of Phage Display Library to Detect Bispecific Antibodies

The growth and development of lymphatic vessels is mediated by VEGF-C binding to VEGFR-3 on the surface on the surface of lymphatic endothelial cells inducing the growth, migration and survival of lymphatic endothelial cells. Invasion of lymphatic vessels into solid tumors promotes growth and spread of these tumors. A method to simultaneously inhibit both VEGF-C and VEGFR-3 activity would provide improved therapies to cancer patients.

To identify inhibitory human monoclonal antibodies against VEGFR-3 or VEGF-C, a human single-chain antibody-phage display library (VTT Biotechnology) was screened. Polyclonal IgM V_{H} and V_{L} kappa and IgM V_{H} and V_{L} lambda phage display libraries were screened for binding to VEGF-C and/or VEGFR-3 using several rounds of panning to the respective molecule. The binding in each round of panning was measured by absorbance (Abs) at 405 nm. Results showed that binding of growth factor or receptor to the respective library of antibodies increased; with each round of panning.

Briefly, for panning, 96-well plates (Greiner) were coated with 1 μg of purified VEGF-C preparation in 100 μl of PBS and incubated overnight at 4° C. The plates were then washed three times with 200 μl of PBS, and blocked with 0.5% BSA in PBS (200 μl) at room temperature (RT) for 2 hours. Phagemid particles (˜1×10¹²) in 3% BSA-PBS were added and incubated at RT for 2 hours. The wells were washed twice with 300 μl PBS, and then an automated wash program was employed (P1: 3×300 μl of PBS with 0s incubation, P2: 5×350 μl of PBS with 5 s incubation and P3: 5×350 μl of PBS with 30 s incubation). The bound phage were eluted with soluble VEGF-C (1 μg/100 μl) overnight at 4° C. The eluted phage (100 μl) were used to infect 3 ml of exponential phase XL-1 Blue cell culture in SB broth containing 10 μg/ml tetracycline and 20 μg/ml carbenicillin. Cells were plated on LB agar containing 100 μg/ml ampicillin and incubated at 37° C. overnight. The next day the colonies were counted on plates to determine the number of infections, and 10 ml of infected bacterial culture was grown by shaking (250 rpm) for 1 hour at 37° C. followed by addition of carbenecillin to the final concentration of 50 μg/ml. The cell culture was further incubated at 37° C. shaker for 1 hour and 1 ml of helper phage VSCM13 (˜10¹²) were added and incubated without shaking at 37° C. for 15 min. To the infected cell culture, 89 ml of SB (50 μg/ml carbenicillin and 10 μg/ml tetracycline) was added and further incubated at 37° C. for 2 hours. After 2 hours of incubation, kanamycin was added to the final concentration of 70 μg/ml and the culture was incubated overnight at 34° C. to produce phage particles. Phage-antibody particles were concentrated from the culture supernatant following precipitation with 25 ml polyethylene glycol in 2.5 M NaCl (20% [w/v]) by centrifugation (Griffits et al., 1994). The phage particle precipitation was repeated once more and the panning cycle was repeated an additional two times.

From the initial panning regimen, sixteen monoclonal anti-VEGF-C binding single chain antibody fragments (scFv) were identified. Each clone was then screened for binding to other growth factors using protein ELISA. Plates were coated with VEGF-C, VEGF-A or VEGF-E and scFv binding affinity was measured by Abs at 405 nm.

Briefly, flat bottom 96-well Immulon® 4 ELISA plates (Dynex Technologies) were coated with 100 μl of different growth factors (10 μg/ml) in PBS overnight at 4° C. The plates were washed 3 times with 300 μl of PBS and then blocked for 1 hour at RT with 300 μl of 0.5% BSA. After washing as previously with PBS, 50 μl of small scale production of scFv were added in wells. The plates were incubated for 2 hours at RT followed by three washes with 300 μl of PBS per well 50 μl of the primary antibody dilution (1:11000)(mouse anti-myc) was added to the wells, and incubated for 1 hour at RT. The plates were then washed as previously and incubated for an additional 1 hour at RT with 50 μl of the secondary antibody, goat anti-mouse phosphatase conjugate (Bio-Rad), diluted 1:2000 in PBS. The plates were washed 3 times with 300 μl PBS and then developed with 50 μl 2 mg/ml 4-nitro-phenylphosphate AFOS (Sigma). The absorbance at 405 nm was measured.

Eight of the sixteen clones (L2F, L10E, L4H, K12C, L4G, L3D, K9F and 5C) bound VEGF-C, VEGF-A and VEGF-E, but appeared to a have higher affinity towards VEGF-C. These scFv antibody genes were sequenced and four isolates (L2F, 5C, K9F, K12C) were subcloned into expression vectors to generate Fab fragments. Binding of the Fab fragments to VEGF-C and VEGF-A was measured using an ELISA as described above. Results demonstrated that all the Fab fragment clones bind both growth factors, but showed a greater affinity for binding to VEGF-C. Antibody heavy and light chain genes from selected clones (K9F and K12C) were amplified using appropriate primers and the products sequenced and digested with compatible enzymes. The scFv genes were cloned into similarly digested soluble expression vector (pKKtac/MCS/laqIq designed and provided by VTT Biotechnology, Finland) and transformed by heat shock into E. coli RV308 for Fab production.

Using the panning technique described above, seven VEGFR-3 specific single chain antibody fragments were isolated (clones 2op.4E, 2op.2G, 3.8H, 2.4C and 3p.5C recognize VEGFR-3 while clones 3p.2G and 3op.7B recognize VEGFR-1, VEGFR-2 and VEGFR-3 equally well). These antibody genes were sequenced and four isolates were subcloned into an expression vector to produce Fab fragments. VEGF-C and VEGFR-3 binding Fab fragments were analyzed in a cell survival assay to measure antibody inhibition of VEGF-C/VEGFR-3 binding. A neutralizing anti-VEGF-C antibody interferes with VEGFR-3 activation thereby inhibiting cell survival.

All cell survival experiments were carried out using a previously established in house bioassay as described in Makinen et al., Nat Med. 7:199-205, 2001. Briefly, Ba/F3 pre-B cells which have been transfected with plasmid constructs encoding chimeric receptors consisting of the extracellular domain of VEGFR-2 or VEGFR-3 fused to the cytoplasmic domain of the erythropoietin (EPO) receptor were used (Stacker, et al., J. Biol. Chem. 274:34884-34892, 1999; Achen, et al., Eur. J. Biochem. 267:2505-2515, 2000). These cells are routinely passaged in interleukin-3 (IL-3) and will die in the absence of IL-3. However, if signaling is induced from the cytoplasmic domain of the chimeric receptors, these cells survive and proliferate in the absence of IL-3. Such signaling is induced by ligands which bind and cross-link the VEGFR extracellular domains.

Media from the anti-VEGF-C Fab producing clone K12C was incubated with cells requiring activation of a VEGFR-3/EpoR fusion for growth. Serial dilutions of the K12C antibody were cultured with the growth factor dependent cell line. At a 1/4 dilution cell viability was approximately 55%, declining to approximately 50% at dilutions of 1/8 and 1/16. Further dilutions of 1/32, 1/64 and 1/128 gave cell viability of approximately 75%, approximately 100%, and approximately 75%, respectively.

By inhibiting growth factor-mediated (e.g., VEGF-C or VEGF-D) stimulation of VEGFR-3, lymphatic growth can be inhibited and lymphatic tumor metastasis reduced. Bispecific antibodies against VEGF-C or VEGFR-3 provide a means for inhibiting multiple factors which mediate aberrant lymphatic development and are useful therapeutics for anti-lymphangiogenic treatments in cancer patients.

### EXAMPLE 13

### Animal Models to Demonstrate the Efficacy of Bispecific Antibody Therapies for Treatment of Cancers

It is contemplated that any accepted animal model for cancer therapies would be useful to demonstrate the efficacy of PDGF/VEGF bispecific antibody substances as therapies for cancer treatment. Exemplary models for demonstrating the efficacy for treatment of breast cancers, using standard dose-response studies, include those described in Tekmal and Durgam, (Cancer Lett., 118: 21-28, 1997); Moshakis et al., (Br. J. Cancer, 43: 575-580, 1981); and Williams et al., (J. Nat. Cancer Inst., 66: 147-155, 1981).

Also, experimental models known in the art to induce development of metastatic tumors are useful for assessing the effects of bispecific antibodies against VEGF-C/VEGF-D on tumor metastasis. Animal models of cancer metastasis include animal models for gastric cancer (Illert et al., Clin. Exp. Metastasis 20:549-54, 2003), colon cancer (Sturm et al., Clin. Exp. Metastasis 20:395-405, 2003), pancreatic cancer (Katz et al., J. Surg. Res. 113:151-60, 2003), prostate cancer (Bastide et al., Prostate Cancer Prostatic Dis. 5:311-15, 2002), lymphogenic metastasis (Dunne et al., Anticancer Res. 22:3273-9, 2002), human lymphoma metastasis (Aoudjit, et al., J. Immunol., 161:2333-2338, 1998), breast cancer (Li et al., Clin. Exp. Metastasis 19:347-56, 2002; Pulaski et al., Cancer Res. 60:2710-15, 2000), colorectal cancer (Kuruppu et al., J Gastroenterol. Hepatol. 13:521-7, 1998), hepatocellular carcinoma, (Lindsay et al., Hepatology 26:1209-15, 1997), neuroblastoma (Engler et al., Cancer Res. 61:2968-73, 2001), and fibrosarcoma metastasis (Shioda et al., J. Surg. Oncol. 64:122-6, 1997; Culp et al., Prog. Histochem. Cytochem. 33:XI-XV, 329-48, 1998).

As an example, mice are inoculated with 7×10³ breast tumor cells or wild type cells as described in Pulaski et al. (supra). Primary breast tumors allowed to grow in an animal for 2-3 weeks typically demonstrate metastasis in the lung, lymph node and liver. The percentage of cells invading these sites increases over time. The tumors are allowed to grow for 2-3 weeks and animals are then treated with appropriate doses, pre-determined by one of skill in the art, with control antibody, monoclonal antibody specific for only one growth factor, or antibody substances specific for more than one PDGF/VEGF growth factor, for example antibodies bispecific for either, VEGF-C/VEGF-D, VEGF-A/VEGF-E, VEGF-A/VEGF-B, VEGF-B/PlGF, or PDGF-C/PDGF-D. Animals are then measured for extent of tumor metastasis after treatment. Tumors may also be excised and the effects of these antibody substances on angiogenesis and lymphangiogenesis at the tumor site is assessed as described above. Antibody substances of the invention are expected to reduce angiogenesis and inhibit tumor growth and metastasis relative to monoclonal or control antibodies.

In addition to murine models, dog and pig models are contemplated because certain antibodies may also recognize growth factors from dog and pig. Tumor size and side effects are monitored to demonstrate therapeutic efficacy versus controls.

### EXAMPLE 14

### Administration of Bispecific Antibody Compositions to Cancer Patients

Administration of antibody substances of the invention in animal models of tumor metastasis provides the basis for administering to cancer patients antibody substances of the invention alone or in combination with cytokines or growth factors, chemotherapeutic agents, radiotherapeutic agents or radiation therapy. An antibody substance is administered using regimens similar to those described for administration of the anti-VEGF antibody (Cobleigh et al., Semin. Oncol. 30(Suppl 16):117-24, 2003; Yang et al., New Engl. J. Med. 349:4278-34, 2003).

PDGF/VEGF specific antibody substances contemplated by the invention are administered to patients within a dose range of 1 mg/kg to 20 mg/kg per treatment. It is recognized by one of skill in the art that the amount of dose will vary from patient to patient, and may be anywhere from 1 mg/kg/day to 100 mg/kg/day. An antibody substance of the invention is administered in doses appropriate for the patient's size, sex, and weight, as would be known or readily determined in the art. Subsequent doses of the antibody substance is increased or decreased to address the particular patient's response to therapy.

An antibody substance is given in any formulation recognized in the art to allow the composition to diffuse into the bloodstream or tissue sites, e.g. aqueous solution or oily suspension. An antibody substance is administered at a frequency and dose determined by the treating physician. For example, antibody substance may be administered once daily for 7 days, twice daily for 7 days, every other day for 14 days, continuously for 14 days, 1 time/week, 1 time every other week, or any other regiment the physician prescribes. An antibody substance may be administered continuously, e.g., through intravenous delivery or by slow release methods, for an extended period of time. The administration may last 1-24 hours, or longer and is amenable to optimization using routine experimentation. The antibody substance may also be given for a duration not requiring extended treatment. Additionally, antibody substances may be administered daily, weekly, bi-weekly, or at other effective frequencies, as would be determinable by one of ordinary skill in the art.

It is contemplated that an antibody substance is administered to patients in combination with other therapeutics, such as with other chemotherapeutic or radiotherapeutic agents, or with growth factors or cytokines. When given in combination with another agent, the amount of antibody substance given may be reduced accordingly. Second agents are administered in an amount determined to be safe and effective at ameliorating human disease.

It is contemplated that cytokines or factors, and chemotherapeutic agents or radiotherapeutic agents are administered in the same formulation as antibody substance and given simultaneously. Alternatively, the agents may also be administered in a separate formulation and still be administered concurrently with bispecific antibody. As used herein, concurrently refers to agents given within 30 minutes of each other. The second agent may also be administered prior to administration of antibody substance. Prior administration refers to administration of the agent within the range of one week prior to antibody substance treatment up to 30 minutes before administration of antibody substance. It is further contemplated that the second agent is administered subsequent to administration of antibody substance. Subsequent administration is meant to describe administration from 30 minutes after antibody substance treatment up to one week after antibody substance administration. Antibody substances may also be administered in conjunction with a regimen of radiation therapy as prescribed by a treating physician.

In one approach, the effectiveness of antibody substance treatment is determined by computer tomographic (CT) scans of the tumor area with the degree of tumor regression assessed by measuring the decrease in tumor size. Biopsies or blood samples are also used to assess the presence or absence and metastasizing ability of particular cell types in response to treatment with antibody substance alone, or in combination with other chemotherapeutic agents. These response assessments are made periodically during the course of treatment to monitor the response of a patient to a given therapy.

A decrease in tumor size, reduction of tumor metastasis and improvement in patient prognosis after treatment with PDGF/VEGF specific antibody substance of the invention alone or in combination with a cytokine or growth factor, a chemotherapeutic agent or a radiotherapeutic agent indicates that the method effectively treats patients exhibiting solid tumor and/or tumors capable of tumor metastasis.

Numerous modifications and variations in the invention as set forth in the above illustrative examples are expected to occur to those skilled in the art. Consequently only such limitations as appear in the appended claims should be placed on the invention.

1. An antibody substance that specifically binds to first and second growth factors selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E), human placental growth factor (PlGF), human platelet-derived growth factor-A (PDGF-A), human platelet-derived growth factor-B (PDGF-B), human platelet-derived growth factor-C (PDGF-C), and human platelet-derived growth factor-D (PDGF-d),
wherein each of said growth factors binds and stimulates phosphorylation of at least one receptor tyrosine kinase, and
wherein the antibody substance inhibits the first and second growth factors to which it binds from stimulating phosphorylation of said receptor tyrosine kinases.

2. An antibody substance produced by a process comprising:
(a) screening a library of antibody molecules to identify at least one antibody molecule that binds to a first growth factor selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E), human placental growth factor (PlGF), human platelet-derived growth factor-A (PDGF-A), human platelet-derived growth factor-B (PDGF-B), human platelet-derived growth factor-C (PDGF-C), and human platelet-derived growth factor-D (PDGF-D),
wherein each of said growth factors binds and stimulates phosphorylation of at least one receptor tyrosine kinase;
(b) screening molecule(s) identified in (a) to identify at least one molecule that binds to a second growth factor selected from said group;
(c) screening molecule(s) identified in step (b) to identify at least one molecule that inhibits the first and second growth factors to which it binds from stimulating phosphorylation of said receptor tyrosine kinases,
wherein the antibody substance comprises a molecule identified in step (c).

3. An antibody substance produced by a process comprising:
(a) screening a library of antibody molecules to identify at least one antibody molecule that binds to a first growth factor selected from the group consisting of human vascular endothelial growth factor-A (VEGF-A), human vascular endothelial growth factor-B (VEGF-B), human vascular endothelial growth factor-C (VEGF-C), human vascular endothelial growth factor-D (VEGF-D), human vascular endothelial growth factor-E (VEGF-E), human placental growth factor (PlGF), human platelet-derived growth factor-A (PDGF-A), human platelet-derived growth factor-B (PDGF-B), human platelet-derived growth factor-C (PDGF-C), and human platelet-derived growth factor-D (PDGF-D),
wherein each of said growth factors binds and stimulates phosphorylation of at least one receptor tyrosine kinase;
(b) screening a library of antibody molecules to identify at least one molecule that binds to a second growth factor selected from said group;
(c) fusing an antigen binding domain of an antibody molecule identified in step (a) with an antigen binding domain of an antibody molecule identified in step (b) to make antibody fusions, and
(d) screening the antibody fusions to identify at least one molecule that inhibits the first and second growth factors to which it binds from stimulating phosphorylation of said receptor tyrosine kinases,
wherein the antibody substance comprises an antibody fusion identified in step (d).

4. The antibody substance of claim 1, wherein the antibody substance comprises an antibody variable region of an antibody that binds the first growth factor attached to an antibody variable region of an antibody that binds the second growth factor.

5. The antibody substance of claim 1, wherein the antibody substance comprises:
antibody heavy and light chain variable regions of an antibody that binds to the first growth factor attached to antibody heavy and light chain variable regions of an antibody that binds to the second growth factor.

6. The antibody substance of claim 5, wherein the antibody heavy and light chain variable regions that bind to the first growth factor are attached to each other to form a single polypeptide; and the antibody heavy and light chain variable regions that bind to the second growth factor are attached to each other to form a single polypeptide.

7. The antibody substance of claim 1, wherein a single polypeptide comprises the antigen binding portions of the antibody substance.

8. The antibody substance of claim 1, wherein said antibody substance comprises a F(ab) antibody fragment that binds to the first growth factor attached to a F(ab) fragment that binds to the second growth factor.

9. The antibody substance of claim 1 wherein said antibody substance comprises a F(ab)₂ fragment that binds to the first growth factor attached to a F(ab)₂ fragment that binds to the second growth factor.

10. The antibody substance of claim 1 that is a monoclonal antibody.

11. The antibody substance of claim 1 that is a single domain antibody that binds to the first growth factor and the second growth factor.

12. The antibody substance of claim 11 wherein the single domain is an Ig variable heavy (V_{H}) chain domain.

13. The antibody substance of claim 11 wherein the single domain is an Ig variable light chain (V_{L}) domain.

14. The antibody substance of claim 1 that is a chimeric antibody.

15. The antibody substance of claim 1 that is a humanized antibody.

16. The antibody substance of claim 1 that is a human antibody.

17. The antibody substance of claim 1, comprising:
(a) a humanized or human antibody heavy chain variable region of an antibody that binds to the first growth factor
(b) a humanized or human antibody light chain variable region of an antibody that binds to the first growth factor;
(c) a humanized or human antibody heavy chain variable region of an antibody that binds to the second growth factor; and
(d) a humanized or human antibody light chain variable region of an antibody that binds to the second growth factor.

18. An antibody substance of claim 17, further comprising human antibody constant regions.

19. The antibody substance of claim 1, wherein the antibody substance comprises a first leucine zipper linked to an antigen binding site that binds the first growth factor, and a second leucine zipper linked to an antigen binding site that binds to the second growth factor, wherein the leucine zippers dimerize to form the antibody substance.

20. A bispecific antibody substance according to claim 1, comprising a first antigen binding site that specifically binds to the first growth factor that is VEGF-B, and a second antigen binding site that specifically binds to the second growth factor that is PlGF, wherein the bispecific antibody substance inhibits VEGF-B-mediated and PlGF-mediated phosphorylation of VEGFR-1.

21. A bispecific antibody substance according to claim 1, comprising a first antigen binding site that specifically binds to the first growth factor that is VEGF-A, and a second antigen binding site that specifically binds to the second growth factor that is VEGF-B, wherein the bispecific antibody substance inhibits VEGF-A-mediated and VEGF-B-mediated phosphorylation of VEGFR-1.

22. A bispecific antibody substance according to claim 21 that inhibits VEGF-A-mediated phosphorylation of VEGFR-2.

23. The bispecific antibody substance according to claim 21 which binds to an epitope comprised of amino acids 87-110 of VEGF-A (SEQ ID NO: 23), amino acids 82-105 of VEGF-B (SEQ ID NO: 24), or a fragment thereof.

24. A bispecific antibody substance according to claim 1, comprising a first antigen binding site that specifically binds to the first growth factor that is VEGF-C and a second antigen binding site that specifically binds to the second growth factor that is VEGF-D, wherein the bispecific antibody substance inhibits VEGF-C-mediated and VEGF-D-mediated phosphorylation of VEGFR-3.

25. A bispecific antibody substance according to claim 24 that inhibits VEGF-C-mediated and VEGF-D-mediated phosphorylation of VEGFR-2.

26. A bispecific antibody substance according to claim 1, comprising a first antigen binding site that specifically binds to the first growth factor that is VEGF-A, and a second antigen binding site that specifically binds to the second growth factor that is VEGF-E, wherein the bispecific antibody substance inhibits VEGF-A-mediated and VEGF-E-mediated phosphorylation of VEGFR-2.

27. A bispecific antibody substance according to claim 1, comprising a first antigen binding site that specifically binds to the first growth factor that is PDGF-A, PDGF-B, PDGF-C or PDGF-D and a second antigen binding site that specifically binds to the second growth factor that is PDGF-A, PBGF-B, PDGF-C or PDGF-D.

28. A bispecific antibody substance according to claim 1, comprising a first antigen binding site that specifically binds to the first growth factor that is PDGF-C and a second antigen binding site that specifically binds to the second growth factor that is PDGF-D, wherein the bispecific antibody substance inhibits PDGF-C-mediated and PDGF-D-mediated phosphorylation of PDGF receptors.

29. The bispecific antibody substance according to claim 28 which binds to an epitope comprised of amino acids 231-274 of PDGF-C (SEQ ID NO: 27), amino acids 255-296 of PDGF-D (SEQ ID NO: 28), or a fragment thereof.

30. The bispecific antibody of claim 29 wherein the epitope comprises amino acids 255-272 of PDGF-D (SEQ ID NO: 29), amino acids 231-250 of PDGF-C (SEQ ID NO: 32), or a fragment thereof.

31. The bispecific antibody of claim 28 which inhibits processing of PDGF-C or PDGF-D to the active state of the protein.

32. A method for inhibiting fibrosis comprising:
administering to a mammalian subject in need of inhibition of fibrosis an antibody substance according to any one of claims 27-30, in an amount effective to inhibit fibrosis.

33. The method of claim 32 wherein the fibrosis is liver fibrosis, cardiac fibrosis, kidney fibrosis or myelofibrosis.

34. The method of claim 32 wherein the subject is human.

35. A composition comprising an antibody substance according to claim 1 in a pharmaceutically acceptable carrier.

36. Use of an antibody according to claim 1 in the manufacture of a medicament for inhibition of angiogenesis or lymphangiogenesis.

37. An isolated polynucleotide comprising a nucleotide sequence that encodes the antibody substance of claim 1.

38. An expression vector comprising the polynucleotide according to claim 37.

39. A host cell transformed or transfected with the polynucleotide according to claim 37 or an expression vector comprising the polynucleotide.

40. A host cell according to claim 39 that expresses the antibody substance encoded by the polynucleotide.

41. A method for producing an antibody substance comprising culturing a host cell according to claim 40 in a culture medium, and recovering the antibody substance from the culture medium.

42. A hybridoma that expresses the monoclonal antibody of claim 10.

43. A method of screening an antibody substance for growth factor neutralization activity comprising:
contacting a growth factor and a growth factor receptor in the presence and absence of an antibody substance; and
measuring binding between the growth factor and the growth factor receptor in the presence and absence of the antibody substance, wherein reduced binding in the presence of the antibody substance indicates growth factor neutralization activity for the antibody substance;
wherein the growth factor comprises at least one member selected from the group consisting of VEGF-A, VEGF-B, VEGF-C, VEGF-D, VEGF-E, PlGF, PDGF-A, PDGF-B, PDGF-C, and PDGF-D and combinations thereof;
wherein the receptor is at least one member selected from the group consisting of VEGFR-1, VEGFR-2, VEGFR-3, PDGFR-α, PDGFR-β; an extracellular domain fragment of any of said receptors that is effective to bind to the growth factor; a chimeric receptor comprising the extracellular domain fragment; and combinations thereof;
wherein the antibody substance comprises an antibody substance according to claim 1.

44. A method according to claim 43, wherein contacting is performed in a cell free system and the measuring of the binding comprises: measuring growth factor bound to the growth factor receptor.

45. A method according to claim 43, wherein the contacting comprises contacting a cell that expresses the receptor with the growth factor; and wherein the measuring comprises:
measuring growth factor receptor phosphorylation, wherein the phosphorylation is indicative of binding;
measuring a growth factor-mediated cellular response in the cell, wherein the cellular response is indicative of binding between the growth factor and the receptor.

46. A method for inhibiting angiogenesis or lymphangiogenesis comprising:
administering to a mammalian subject in need of inhibition of angiogenesis or lymphangiogenesis an antibody substance according to claim 1, in an amount effective to inhibit angiogenesis or lymphangiogenesis.

47. A method according to claim 46, wherein the subject has a disease characterized by neoplastic cell growth with exhibiting angiogenesis or lymphangiogenesis, and the antibody substance is administered in an amount effective to inhibit the neoplastic cell growth.

48. The method of claim 46 wherein the subject has a disease characterized by aberrant angiogenesis or lymphangiogenesis, wherein the disease is selected from the group consisting of inflammation (chronic or acute), an infection, an immunological disease, arthritis, rheumatoid arthritis, diabetes, retinopathy, psoriasis, arthopathies, congestive heart failure, fluid accumulation due to vascular permeability, lymphangioma, and lymphangiectasis.

49. The method of claim 46 where the subject is human.

50. A method of inhibiting neoplastic cell growth comprising steps of:
(a) diagnosing a mammalian subject with neoplastic cell growth
(b) assaying the neoplastic cell growth for expression of two or more growth factors selected from the group consisting of VEGF-A, VEGF-B, VEGF-C, VEGF-D, VEGF-E, PlGF, PDGF-A, PDGF-B, PDGF-C, and PDGF-D,
(c) administering to the subject an antibody substance according to claim 1, wherein the antibody substance binds two or more growth factors identified in step (b) as being expressed in the neoplastic cell growth.

51. A method according to claim 50, wherein the neoplastic cell growth is a tumor.

52. A method of inhibiting neoplastic cell growth, comprising steps of:
(a) diagnosing a mammalian subject with neoplastic cell growth
(b) assaying the neoplastic cell growth for expression of at least one tyrosine kinase receptor selected from the group consisting of VEGFR-1, VEGFR-2, VEGFR-3, PDGFR-alpha, and PDGFR-beta; and
(c) administering to the subject an antibody substance according to claim 1, wherein the antibody substance binds to two or more growth factors that bind to at least one receptor tyrosine kinase identified in step (b) as being expressed in the neoplastic cell growth.

53. The method of claim 46, further comprising administering to the subject a treatment selected from the group consisting of a chemotherapeutic agent, a radiotherapeutic agent, or radiation therapy.


SEQUENCE LISTING (32 sequences)

SEQ ID NO: 1 - DNA, 649 bp, Homo sapiens
tcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagcgtttcgggaaccagatctctcaccagg

SEQ ID NO: 2 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 1645 bp, Homo sapiens
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 4 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 5 - DNA, 755 bp, Homo sapiens
caccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaa

SEQ ID NO: 6 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 7 - DNA, 2076 bp, Homo sapiens
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 8 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 9 - DNA, 2128 bp, Homo sapiens
caagacttctctgcattttctgccaaaatctgtgtcagatttaagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 10 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 11 - DNA, 3007 bp, Homo sapiens
gcccggagagccgcatctattggcagctttgttattgatcagaaactgctcgccgccgacttggcttccagtctggctgcgggcaacccttgagttttcgcctctgtcctgtcccccgaactgacaggtgctcccagcaacttgctggggacttctcgccgctcccccgcgtccccaccccctcattcctccctcgccttcacccccacccccaccacttcgccacagctcaggatttgtttaaaccttgggaaactggttcaggtccaggttttgctttgatccttttcaaaaactggagacacagaagagggctctaggaaaaagttttggatgggattatgtggaaactaccctgcgattctctgctgccagagcaggctcggcgcttccaccccagtgcagccttcccctggcggtggtgaaagagactcgggagtcgctgcttccaaagtgcccgccgtgagtgagctctcaccccagtcagccaaatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcgtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatgatataaaatattgttgctctgacaaaaatacatgtatttcattctcgtatggtgctagagttagattaatctgcattttaaaaaactgaattggaatagaattggtaagttgcaaagactttttgaaaataattaaattatcatatcttccattcctgttattggagatgaaaataaaaagcaacttatgaaagtagacattcagatccagccattactaacctattccttttttggggaaatctgagcctagctcagaaaaacataaagcaccttgaaaaagacttggcagcttcctgataaagcgtgctgtgctgtgcagtaggaacacatcctatttattgtgatgttgtggttttattatcttaaactctgttccatacacttgtataaatacatggatatttttatgtacagaagtatgtctcttaaccagttcacttattgtactctggcaatttaaaagaaaatcagtaaaatattttgcttgtaaaatgcttaatatcgtgcctaggttatgtggtgactatttgaatcaaaaatgtattgaatcatcaaataaaagaatgtggctattttggggagaaaatt

SEQ ID NO: 12 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 13 - DNA, 2253 bp, Homo sapiens
cgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgcgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaaggtttccagagaaaaggccaatacctaagcattttttccatgagaagcactgcatacttacctatgtggactgtaataacctgtctccaaaaccatgccataataatataagtgctttagaaattaaatcattgtgttttttatgcattttgctgaggcatccttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaaccg

SEQ ID NO: 14 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 15 - DNA, 1830 bp, ORF Virus
cggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtggcggtgctcggcgtggtggcaatttcgctgctgcgccgcgcgctaagaatacggtttagatactcaaagtctatccagacacttagagtgtaactttgagtaaaaaatgtaaatactaacgccaaaatttcgatagttgttaagcaatatataacatttttaaaacgtcatcaccagcatgaagttaacagctacgttacaagttgttgttgcattgttaatatgtatgtataatttgccagaatgcgtgtctcagagtaatgattcacctccttcaaccaatgactggatgcgtacactagacaaaagtggttgtaaacctagagatactgttgtttatttgggagaagaatatccagaaagcactaacctacaatataatccccggtgcgtaactgttaaacgatgcagtggttgctgtaacggtgacggtcaaatatgtacagcggttgaaacaagaaatacaactgtaacagtttcagtaaccggcgtgtctagttcgtctggtactaatagtggtgtatctactaaccttcaaagaataagtgttacagaacacacaaagtgcgattgtattggtagaacaacgacaacacctacgaccactagggaacctagacgataactaataacaaaaaatgtttatttttgtaaatacttaattattacacactttacaataatctcaaaaataaattgcgtgcccggacggctgcagctggtgacgctgctgtgtcacacactgcgtattcgattcaagttcactaacgccactaaactagttgtgcgtgtccgagtgttaaccgtacgtcaaactaacatcttacctgtccgtgacaagaactaaaacttgaaccacatatttttaaagtatatttaacaaaatcactcacactcacacaatcataaacaccacaaccacaaccaaacacgcatgagaattaatattcttacttatccgtaacactctatgctgtacatcaacgcatcagagcagtctgagtctgactaatggcggcaaacgggaacgcaggcgcgacataatcactgagaatctccgcagcaaccgctcaaggacatctctagcgctaacggctgtttgtcattcccccgtgtgttcatctcacacgacattgtgaccgtcgcaaagcacacattcaaagtgccgcatgtggaagaattcaccgtcgagacacacaccataattaaacaagatcagtgcataagagagattagcattctacagcacaccacgtgcgaatacggacctcgtaattgtttagactagaacacctctggtctaaacaacatgtccgatcttagaacagagtttatgacgcatatgtaactgtgttctttatgtagaagttatcttttatgtcactcccttgtcttagatgagttatacatgacatgatgtatgtgtcgcccgcggcggcgcggggcgctcggcggcggggctgctgcgcgcggcgggcccgcggtggcggcggctggcgcggcgctgcggccgcgggcgcgcggcggggtagcggcccgcccgcccgggcgcccgccgcagcccttgccccggaccaggcgccacggagcaaagtgaaaaaggaccgcctagcagtcgagaccctcccgccgcagccgcgacaccccacacccgccttccacccgccagacgccaacaccacagccaacaagcatgc

SEQ ID NO: 16 - protein, 148 aa, ORF Virus
MKLTATLQVVVALLICMYNLPECVSQSNDSPPSTNDWMRTLDKSGCKPRDTVVYLGEEYPESTNLQYNPRCVTVKRCSGCCNGDGQICTAVETRNTTVTVSVTGVSSSSGTNSGVSTNLQRISVTEHTKCDCIGRTTTTPTTTREPRR

SEQ ID NO: 17 - DNA, 2797 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgttcaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 18 - protein, 211 aa, Homo sapiens [sequence omitted]

SEQ ID NO: 19 - DNA, 3373 bp, Homo sapiens
ggtggcaacttctcctcctgcggccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcaggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagccggcctcggacccaggtcctgcgcccaggtcctcccctgccccccagcgacggagccggggccgggggcggcggcgccgggggcatgcgggtgagccgcggctgcagaggcctgagcgcctgatcgccgcggacctgagccgagcccacccccctccccagccccccaccctggccgcgggggcggcgcgctcgatctacgcgtccggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggtcttgcccagcagctcaaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggtgtagatggtgacctgggtatcccctgcctcctgccaccccttcctccccatactccactctgattcacctcttcctctggttcctttcatctctctacctccaccctgcattttcctcttgtcctggcccttcagtctgctccaccaaggggctcttgaaccccttattaaggccccagatgaccccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggctggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgt

SEQ ID NO: 20 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 21 - DNA, 399 bp, ORF Virus
atgaagttgctcgtcggcatactggtagccgtgtgcttgcaccagtatctgctgaacgcggacagcacgaaaacatggtccgaggtgtttgaaagcagtaagtgcaagccaaggccaacggtcgttcccgtaggcgaggcgcacccagagctaacttctcagcggttcaacccgcagtgtgtcacagtgatgcgatgcggcgggtgctgcaacgacgagagcttggaatgcgtccccacggaagaggcaaacgtgacgatgcaactcatgggggcgtcggtctccggtggtaacgggatgcaacatttgatattcgtagagcataagaaatgcgattgtaaaccacgactcacaaccacgccaccgacgaccacaaggccgcccagaagacgccgctag

SEQ ID NO: 22 - protein, 132 aa, ORF Virus
MKLLVGILVAVCLHQYLLNADSTKTWSEVFESSKCKPRPTVVPVGEAHPELTSQRFNPQCVTVMRCGGCCNDESLECVPTEEANVTMQLMGASVSGGNGMQHLIFVEHKKCDCKPRLTTTPPTTTRPPRRRR

SEQ ID NO: 23 - protein, 24 aa, Homo sapiens
CNDEGLECVPTEESNITMQIMRIK

SEQ ID NO: 24 - protein, 24 aa, Homo sapiens
CPDDGLECVPTGQHQVRMQILMIR

SEQ ID NO: 25 - DNA, 33 bp, Homo sapiens
cgcggatccgaagaggtaaaactctacagctgc

SEQ ID NO: 26 - DNA, 29 bp, Homo sapiens
ggaattccccctcctgcgtttcctctaca

SEQ ID NO: 27 - protein, 44 aa, Homo sapiens
RKSRVVDLNLLTEEVRLYSCTPRNFSVSIREELKRTDTIFWPGC

SEQ ID NO: 28 - protein, 43 aa, Homo sapiens
RKSKVDLDRLNDDAKRYSCTPRNYSVNIREELKLANVVFFPRC

SEQ ID NO: 29 - protein, 19 aa, Homo sapiens
CKSKVDLDRLNDDAKRYSC

SEQ ID NO: 30 - DNA, 19 bp, Artificial sequence
  Synthetic primer
taatggatccggcaggtca

SEQ ID NO: 31 - DNA, 20 bp, Artificial sequence
  Synthetic primer
taatctcgagctcgaggtgg

SEQ ID NO: 32 - protein, 20 aa, Homo sapiens
RKSRVVDLNLLTEEVRLYSC